(19) United States
(12) Patent Application Publication
(10) Pub. No.: US 2008/0019950 A1
(43) Pub. Date: Jan. 24, 2008

(54) Novel hepatocyte-like cells and hepatoblast-like cells derived from hBS cells

(75) Inventors: Nico Heins, Vastra Frolunda (SE); Barbara Kuppers-Munther, Gothenburg (SE); Josefina Edsbagge, Torslanda (SE)

Correspondence Address: BUCHANAN, INGERSOLL & ROONEY PC, POST OFFICE BOX 1404, ALEXANDRIA, VA, 22313-1404, US

(73) Assignee: CELLARTIS AB, Gotenburg (SE)

(21) Appl. No.: 11/806,822

(22) Filed: Jun. 4, 2007

Related U.S. Application Data
(60) Provisional application No. 60/810,626, filed on Jun. 5, 2006.
(60) Provisional application No. 60/879,802, filed on Jan. 11, 2007.
(60) Provisional application No. 60/924,108, filed on Apr. 30, 2007.

(30) Foreign Application Priority Data
Jun. 5, 2006 (DK) PA 2006 00761
Apr. 30, 2007 (DK) PA 2007 00645
Jun. 4, 2006 (SE) 0601255-3

(51) Int. Cl. A61K 45/00; A61P 3/00; A61P 3/10; A61P 31/12; A61P 37/00; C12N 5/08; C12P 1/00; C12Q 1/02

(52) U.S. Cl. 424/93.7; 435/29; 435/366; 435/41

(57) ABSTRACT

The present invention relates to a novel hepatocyte-like cell population derived from hBS cells and to the potential use of such heopatocyte-like cells in e.g. medical treatment, drug screening and toxicity testing. Furthermore, the invention relates to hepatoblast-like cells that may have suitable characteristics so that they can be used for the same applications as the hepatocyte-like cells and that furthermore may be used in in vitro studies of hepatogenesis such as early hepatogenesis or hepato-regenerative disorders. Both the hepatocyte-like and the hepatoblast-like cells according to the invention express drug transporter and/or drug metabolising characteristics either at the gene or protein expression level.

### FIELD OF THE INVENTION

The present invention relates to a novel hepatocyte-like cell population derived from hBS cells and to the potential use of such hepatocyte-like cells in e.g. medical treatment, drug screening and toxicity testing. Furthermore, the invention relates to hepatoblast-like cells that may have suitable characteristics so that they can be expanded and when needed differentiated further into functional hepatocyte-like cells, and that furthermore may be used for in vitro and in vivo studies of hepatogenesis such as early hepatogenesis or hepato-regenerative disorders. The hepatocyte-like cells according to the invention express drug transporters and/or drug metabolising characteristics either at the gene or protein expression level.

### BACKGROUND OF THE INVENTION

Pluripotent human stem cells are expected to revolutionize the accessibility to a variety of human cell types. The possibility to propagate pluripotent human blastocyst-derived stem (hBS) cells and subsequently differentiate them into the desired target cell types will provide a stable and virtually unlimited supply of cells for a range of applications in vivo and in vitro.

Liver failure and end-stage liver diseases are responsible for a huge amount of deaths around the world and is a major burden on the health care system. Liver transplantation remains the most successful treatment. However, the efficacy of this procedure is limited and connected to many complications such as infection or rejection. Liver transplantation also suffers from shortage of available donor organs and the treated patients will very often be referred to lifelong immunosuppression. By reducing the need for organs, cell-based treatments will be of great importance to both society and to the individuals suffering from these severe diseases.

Furthermore, the liver is the centre of metabolism and detoxification in the human body, and therefore huge efforts have been undertaken in order to identify a reliable source of functional cell types for in vitro testing. Unfortunately, the complexity and function of the liver is not mirrored by any cell type available today. The availability of primary human liver cells is limited and the cells are also known to rapidly loose their normal phenotype and functional properties (i.e. within 24 hours) when used for in vitro applications. One often used alternative to primary cells are hepatic cell lines which in turn contain very low levels of metabolising enzymes and have distributions of other important proteins substantially different from the native hepatocyte in vivo. Thus, many tests are still performed using animal material, even though liver metabolism is known to be species-specific and thereby generating difficulties in predicting liver toxicity in another species than the one tested.

In pharmaceutical development adverse liver reactions remain the most prominent toxicity liability. Therefore early prediction of human liver toxicity liabilities is of paramount importance when selecting compounds to enter clinical trials. Efforts to improve capabilities in this area must address both the availability question and development of models, which provide greater coverage for the complex biological processes which coincide to induce adverse liver injury in human. In both areas the use of differentiated cells derived from hBS cells provide promising opportunities.

Accordingly there is an urgent need for a model system that mimics human liver cells and that is able to predict effects of candidate molecules in the development of new drugs or chemicals. Regarding both availability and physiological relevance human pluripotent stem cells may serve as an ideal renewable source of functional human hepatocytes. When hBS cells have been placed in a proper environment certain hepatic characteristics have been observed after 2-4 weeks of differentiation.

Previous studies by Rambhatla et al 2003, WO 01/81549 and WO 2005/097980 have identified cells with some hepatocyte-like characteristics, i.e. CYP and GST activities in differentiated hBS cell cultures, but so far the cells generated have not shown the metabolic qualities necessary for potentially replacing traditional liver systems in terms of drug transporter expression and specific CYP and GST expression patterns. In the present invention is presented a hBS cell derived hepatocyte-like cell population for use in drug discovery and regenerative medicine with a stable expression for at least 72 hours of important metabolizing enzymes as well as drug transporters.

The Cellartis patent application WO2006034873 is based on a method which allows the use of different factors in a defined manner to follow the paths of developmental biology. In the present invention, it is mainly the secreted intrinsic factors of the cell that affects the differentiation. In addition, the frequency in the change of media differs. The present method allows for less frequent change of media and is therefore a less labor intensive method. Furthermore, the cells of the present invention are more matured and are possible to culture for a longer period of time in assay systems useful for drug-discovery and toxicity testing.

Neither WO2005097980 nor US20030003573 teach about the presence of drug transporters or functional transporters. WO2005097980 only states that CYP3A4, CYP2C9 and CYP1A2 are desirable enzymes for drug screening (see table 3). However, the application does not teach anything about the activity of these most important CYPs. In particular, CYP3A4 is the single most important enzyme for use in drug discovery and toxicity testing. A majority of all drugs are metabolized via CYP3A4. Thus, it would be desirable if a hepatocyte derived from hBS cells exhibited functional CYP3A4, CYP2C9 and CYP1A2 enzymes in an interindividual composition that reflects human adult liver cell.

It would also desirable for drug discovery and toxicity testing if hepatocytes derived from hBS had a combination of (i) functional CYP3A4, CYP2C9 and CYP1A2 enzymes, (ii) functional GST enzymes and (ii) functional drug transporters. There are no details of the cells described in WO200509780 with respect to the most important CYPs and drug transporters, and moreover, it shows limited GSTs characterization data. In contrast, the present invention has a thorough description of the Phase II enzymes.

### BRIEF DESCRIPTION OF THE INVENTION

The present invention relates to hepatocyte-like cells and hepatoblast-like cells, and the methods for their respective preparation. The hepatocyte-like cells of the present invention are especially well suited for use in drug discovery and toxicity testing, because they express drug transporters and/or metabolizing enzymes.

Human blastocysts-derived stem cells (hBS cells) are pluripotent and can give rise to cells of all three embryonic germ layers; endoderm, ectoderm and mesoderm, and further on to all somatic and germ cells. Thus, in the future, differentiated cells derived from hBS cells with functional characteristics of hepatic cells do not only have the potential of being used for transplantation or in bioreactors for extra corporal liver support in patients with liver failure, but also as a test system for studying drug targets, hepatic metabolism of xenobiotics, and hepatotoxicity. hBS derived hepatocytes can potentially provide an unlimited source of functional human hepatocytes, from the same genetic donor if desired, and thereby improve the predictability of in vitro testing such as toxicity tests and reduce the need for animal experimentation. However, the toxicity of xenobiotics is often dependent on their biotransformation into toxic and reactive metabolites and, therefore, the presence and distribution of biotransforming systems are required. At present, primary human hepatocytes constitute a model for in vitro drug metabolism and toxicity testing. Nevertheless, the activity of drug metabolizing enzymes and many transporter functions are rapidly lost and/or changed when primary hepatocytes are cultured. Moreover, many of the hepatoma cell lines, e.g. HepG2, which are used for in vitro studies, lack expression of many important drug metabolizing enzymes.

Cytochrome P450s (CYPs) are mixed function monooxygenases and the major enzymes in phase I metabolism of xenobiotics. This oxidative metabolism results in, depending on the nature of the xenobiotic, inactivation and facilitated elimination, activation of pro-drugs or metabolic activation. The major site of CYP expression is the liver and CYP3A4 is the most abundant CYP isozyme in human adult liver. The enzymes of greatest importance for drug metabolism belong to the families 1-3, responsible for 70-80% of all phase I dependent metabolism of clinically used drugs. CYP expression and activity present large interindividual variations due to polymorphisms. Moreover, CYPs can be induced several fold or inhibited by specific drugs, resulting in additional, although transient, variability of metabolic activity. Notably, the composition of the three major CYP-families (1-3) basal CYP-activity within a hepatocyte is of great importance for drug metabolism. In the examples herein is described hBS cell derived hepatocytes-like cells in which mRNA from most of the CYP enzymes including CYP1A2 and CYP3A4/7 were detected. Basal CYP-activity of the major CYP-families, more precisely CYP1A2, CYP2C9 and CYP3A4, were detected and in addition the interindividual composition of the activity of the three mentioned CYPs was similar to that of human primary hepatocytes. Accordingly, the present invention provides methods for the preparation of hepatocyte-like cells that express functional drug metabolising enzymes.

Functional drug transporters such as BSEP, MRP2 and OATP:s in hepatocytes are essential when analysing drug metabolism and toxicity of the liver. Accordingly, the present invention provides methods for the preparation of hepatocyte-like cells that express functional transporters.

Thus, the present invention relates to a cell population derived from hBS cells, wherein at least 20% of the cells in the cell population exhibit at least one of the following characteristics Alpha-1-Antitrypsin, Cytokeratin 18, HNF-3beta, Albumin or Liver-Fatty-Acid-Binding-Protein and the cell population has at least three of the following six characteristics

A. Drug transporters

i) at least 1% of the cells exhibit protein and/or gene expression of BSEP,
ii) at least 1% of the cells exhibit protein and/or gene expression of MRP2,
iii) at least 1% of the cells exhibit protein and/or gene expression OATP2 and/or OATP-8,
   B. Drug metabolising enzymes
iv) at least 20% of the cells exhibit protein and/or gene expression of GST A1-1,
v) at least 20% of the cells exhibit protein and/or gene expression of at least 2 of the following CYP450s-1A2, -2A6, -2B6, -2C8, -2C9, -2C19-2D6, -2E1, -3A4 and -3A7,
vi) at least 20% of the cells do not exhibit protein and/or gene expression of GST P1-1.

Furthermore, the present invention relates to a cell population derived from hBS cells, wherein at least about 10% of the cells in the cell population express at least one of HNF3beta and AFP and have proliferative capacity and the cell population has at least two of the following four characteristics

A. Receptor

i) at least 1% of the cells exhibit protein and/or gene expression of alpha-6-integrin,
ii) at least 1% of the cells exhibit protein and/or gene expression of c-Met,
   B. Intercellular adhesion molecule
iii) at least 1% of the cells exhibit protein and/or expression of ICAM-1
   C. Transcription factor
iv) at least 10% of the cells exhibit protein and/or expression of HNF-4 alpha.
   D. Cytokeratin
v) at least 1% of cells exhibits protein and/or expression of CK19.
vi) at least 1% of cells exhibits protein and/or expression of CK7.
   E. Epithelial cell adhesion molecule
vii) at least 1% of cells exhibits protein and/or expression of EpCAM.

Urea is the final degradation product of protein and amino acid metabolism. Hepatocytes in the liver are the only cell type of the body to transform ammonia to urea. Accordingly, the present invention provides methods for the preparation of hepatocyte-like cells that synthesize urea.

In one embodiment of the invention, hBSC-derived hepatocyte-like cells produce and secrete urea into the medium at levels similar to primary hepatocytes. The hepatocyte-like cells have the capacity to synthesize at least 10%, 20%, 50%, 70%, 80%, 90% or at least 100% of urea compared to primary hepatocytes. The hepatocyte-like cells can be analysed for urea from day 10 to day 20 and onwards with a remaining high level of urea synthesis. For more details, see Example 10 herein.

Isolation and reseeding of hepatocyte-like cells to different feeder free surfaces enables purified hepatocyte-like cell populations in different formats which is necessary for the flexibility demanded by different applications within drug toxicity and metabolism testing as well as other test assays based on hepatocytes. Accordingly, the present invention provides methods for the preparation of purified and enriched hepatocyte-like cells feeder-free cultures, preferably collagen I cultures, in any format such as 96-well plates.

In one embodiment of the invention, the hepatocyte-like cells are successfully reseeded onto different surfaces of wells, such as a 96-well plate. The different surfaces can be collagen I, Matrigel or mEF cell layer. It is very difficult to reseed primary hepatocytes. Thus, it is a true advantage compared to primary hepatocytes that the hepatocyte-like cells have the ability to be reseeded. For further details, see Example 15 herein.

The ability to keep hepatoblasts in a progenitor state with the ability to expand when cultured under proliferative permissive conditions and differentiate into functional hepatocytes when kept in differentiation suitable conditions would be valuable for keeping un unlimited source of functional hepatocytes. Accordingly, the present invention provides methods for keeping hepatoblast-like cells in a progenitor state by reseeding hepatoblast-like cells on to mEF-cell layer. In addition the hepatoblast-like cells are differentiating into hepatocyte-like cells when reseeded onto matrigel or collagen coated surfaces.

### DESCRIPTION OF THE INVENTION

### DEFINITIONS AND ABBREVIATIONS

As used herein feeder cells are intended to mean supporting cell types used alone or in combination. The cell type may further be of human or other species origin. The tissue from which the feeder cells may be derived include embryonic, fetal, neonatal, juvenile or adult tissue, and it further includes tissue derived from skin, including foreskin, umbilical chord, muscle, lung, epithelium, placenta, fallopian tube, glandula, stroma or breast. The feeder cells may be derived from cell types pertaining to the group consisting of human fibroblasts, fibrocytes, myocytes, keratinocytes, endothelial cells and epithelial cells. Examples of specific cell types that may be used for deriving feeder cells include embryonic fibroblasts, extraembryonic endoderm cells, extraembryonic mesoderm cells, fetal fibroblasts and/or fibrocytes, fetal muscle cells, fetal skin cells, fetal lung cells, fetal endothelial cells, fetal epithelial cells, umbilical chord mesenchymal cells, placental fibroblasts and/or fibrocytes, placental endothelial cells, post-natal foreskin fibroblasts and/or fibrocytes, post-natal muscle cells, post-natal skin cells, post-natal endothelial cells, adult skin fibroblasts and/or fibrocytes, adult muscle cells, adult fallopian tube endothelial cells, adult glandular endometrial cells, adult stromal endometrial cells, adult breast cancer parenchymal cells, adult endothelial cells, adult epithelial cells or adult keratinocytes. When feeder cells are derived from hBS cells, the cells may be fibroblasts.

As used herein, the term “3D” is intended to mean three dimensional.

As used herein, the term “blastocyst-derived stem cell” is denoted BS cell, and the human form is termed “hBS cells”.

As used herein, the term “AAT” is intended to mean the liver marker alpha-anti-trypsin.

As used herein, the term “AFP” is intended to mean the liver marker alpha-feto-protein.

As used herein, the term “BSEP” is intended to mean bile salt export pump.

As used herein, the term “CK” is intended to mean the liver marker cytokeratin (used interchangeably), with different subtypes such as Cytokeratin 18, Cytokeratin 19 and Cytokeratin 7.

As used herein, the term “c-Met” is intended to mean hepatocyte growth factor and/or scatter factor receptor.

As used herein, the term “ICAM-1” is intended to mean intracellular adhesion molecule 1.

As used herein, the term “LFABP” means Liver-Fatty-Acid-Binding-Protein (used interchangeably).

As used herein, the term “EpCAM” means Epithelial Cell Adhesion Molecule (used interchangeably).

As used herein, the term “FGF” means fibroblast growth factor, preferably of human and/or recombinant origin, and subtypes belonging thereto are e.g. bFGF (sometimes also referred to as FGF2) and FGF4.

As used herein, the term “DMSO” means dimethylsulfoxide.

As used herein “CYP” is intended to mean Cytochrome P, and more specifically Cytochrome P 450, the major phase 1 metabolizing enzyme of the liver constituting of many different subunits, such as 1A1, 1A2, 3A4 etc.

As used herein, the term “GST” is intended to mean glutathione transferase, and examples of subtypes thereof are GST A1-1, GST M1-1, and GST P1-1.

As used herein the “HNF3beta”, and/or “HNF3b”, used interchangeably are intended to mean hepatocyte nuclear factor 3, a transcription factor regulating gene expression in endodermal derived tissue, e.g. the liver, pancreatic islets, and adipocytes. HNF3beta may sometimes also be referred to as Foxa2, the name originating from the transcription factor being a member of Forkhead box transcription factors family.

As used herein the term “OATP” is intended to mean Organic Anion Transporting polypeptide, that mediate the sodium (Na+)-independent transport of organic anions, such as sulfobromophthalein (BSP) and conjugated (taurocholate) and unconjugated (cholate) bile acids (by similarity) in the liver.

As used herein the term “UGT” is intended to mean Uridine diphosphoglucuronosyltransferase, which is a group of liver enzymes catalyzing glucuronidation activities.

As used herein the term “xeno-free” is intended to mean complete circumvention of direct or in-direct exposure to non-human animal components.

As mentioned in the above, the present invention provides improved hepatocyte-like cells and hepatoblast-like cells derived from hBS cells. The improved hepatocyte-like cells express drug transporters and/or metabolizing enzymes, ensuring similar drug uptake, secretion and metabolism as liver cells in vivo using the same drug transporters and metabolizing enzymes. Thus, expression of all of these features are desirable features for cells to be used in drug discovery and toxicity testing, as their reaction towards drugs and chemicals are expected to resemble the liver cells in vivo.

Accordingly, the hepatoblast-like cells or the hepatocyte-like cells disclosed in the present invention are advantageously used for a multitude of investigative purposes, such as, e.g., in a drug discovery process, in in vitro models for studying drug transporters, in in vitro models for studying drug metabolizing enzymes, in in vitro models for studying hepatogenesis, such as, e.g., early hepatogenesis, in in vitro models for studying human hepatoregenerative disorders, for in vitro hepatotoxicity testing.

Furthermore, the hepatoblast-like cells and hepatocyte-like cells according to the present invention can advantageously be used for treatment and/or prevention of several hepatic diseases and disorders. Accordingly, the hepatoblast-like cells and hepatocyte-like cells according to the present invention can be used in a medicament.

The hepatoblast-like cells are the progenitor cells of hepatocyte-like cells, and accordingly, they are suitably used e.g. for obtaining metabolically competent hepatocyte-like cells, or for studying the maturation towards hepatocyte-like cells.

Hepatocyte-Like Cells

In the present context, the term “hepatocyte-like cells” is intended to mean cells exhibiting at least one of the following characteristics Alpha-1-antitrypsin, Cytokeratin 18, HNF-3beta, Albumin or Liver-Fatty-Acid-Binding-Protein. The hepatocyte-like cells according to the present invention furthermore have important and stable characteristics relating to drug transport and drug metabolism.

Accordingly, in one embodiment the present invention relates to a cell population derived from hBS cells, wherein at least 20% of the cells in the cell population exhibits at least one of the following characteristics Alpha-1-Antitrypsin (AAT), Cytokeratin 18 (CK18), HNF-3beta, Albumin or Liver-Fatty-Acid-Binding-Protein (LFABP) and the cell population has at least three of the following six characteristics

A. Drug transporters

i) at least 1% of the cells exhibit protein and/or gene expression of BSEP,
ii) at least 1% of the cells exhibit protein and/or gene expression of MRP2,
iii) at least 1% of the cells exhibit protein and/or gene expression OATP2 and/or OATP-8,
   B. Drug metabolising enzymes
iv) at least 20% of the cells exhibit protein and/or gene expression of GST A1-1,
v) at least 20% of the cells exhibit protein and/or gene expression of at least 2 of the following CYP450s-1A2, -2A6, -2B6, -2C8, -2C9, -2C19-2D6, -2E1, -3A4 and -3A7,
vi) at least 20% of the cells do not exhibit protein and/or gene expression of GST P1-1.

In addition or as a substitute for requirement vi) at least 5% of the cells exhibit protein and/or gene expression of GST M1-1.

In one embodiment of the invention, the hepatocyte-like cells can metabolize drugs via the phase I cytochrome p450 enzymes. In particular, cyp1A2, cyp2C9 and cyp3A4 can be metabolized in the absence of inducers. In one embodiment, the substances metabolised by the hepatocyte-like cells are Phenacetin, Diclofenac and Midazolam and the metabolites were analyzed by LC-MS. It is important to note that the hepatocyte-like cells are capable of metabolizing drugs without the influence of inducers (as for example described in WO2005097980).

In a further embodiment, the hepatocyte-like cells have a composition of cyp-activity similar to the cyp activity composition in human primary hepatocyte cultures. Specifically, the composition of Cyp1A2, Cyp3A4 and Cyp2C9 in the hepatocyte-like cells are comparable to the composition in human primary hepatocyte cultures. The Cyp-activity composition between Cyp1A2, Cyp3A4 and Cyp2C9 can differ from 30%, 50%, 75% and 100% compared to the composition in human primary hepatocyte cultures.

In one embodiment of the invention, the hepatocyte-like cells express functional drug transporters. In particular, OATP-2 is active measured by take up of an ICG dye which is an indication of the presence of functional drug transporters within the cells (FIG. 48).

In a further embodiment of the present invention the cell population derived from hBS cells, wherein at least 20% of the cells in the cell population exhibit at least one of the following characteristics Alpha-1-antitrypsin, Cytokeratin 18, HNF-3beta, Albumin or Liver-Fatty-Acid-Binding-Protein and the cell population has the following the two following characteristics

A. Drug transporters

iii) at least 1% of the cells exhibit a functionally active OATP-2 and/or OATP-8
   B. Drug metabolising enzymes
iv) at least 20% of the cells exhibit functional activity of GSTA1-1
v) at least 20% of the cells exhibit a functionally active Cyp1A2, Cyp3A4 and/or Cyp2C9 measured by analyzing the drug metabolites.

In a further embodiment of the present invention a cell population derived from hBS cells, wherein at least 75% of the cells in the cell population exhibit the following characteristics Alpha-1-antitrypsin, Cytokeratin 18, HNF-3beta, Albumin or Liver-Fatty-Acid-Binding-Protein and the cell population has at least the two following characteristics

A. Drug transporters

iii) at least 10% of the cells exhibit a functionally active OATP-2 and/or OATP-8
   B. Drug metabolising enzymes
iv) at least 30% of the cells exhibit functional activity of GSTA1-1
v) at least 50% of the cells exhibit a functionally active Cyp1A2, Cyp3A4 and/or Cyp2C9 measured by analyzing the drug metabolites.

Glycogen storage is another prominent feature of hepatocyte-like cells.

Moreover, a percentage of the hepatocyte-like and hepatoblast-like cells are positive for Notch-2. The Notch signaling pathways are widely used for embryonic development in adults and maintenance of homeostasis. It is also one of the key pathways constituting the stem cell signaling network. In mammals, four Notch receptors (Notch1-Notch4) and five structurally similar Notch ligands (Delta-like1 [also called Delta1], Delta-like3, Delta-like4, Jagged1, and Jagged2) have so far been identified. Notch ligands are single-pass transmembrane proteins. By binding with ligands expressed on adjacent cells Notch receptors are activated, which leads to proteolytic release and nuclear translocation of the intracellular domain of Notch which in turn regulates differentiation. Notch-2 is widely expressed during embryonic development and has a critical role in many organs. In the liver Notch-2 is involved in the formation and differentiation of intrahepatic ducts (Ader et al., 2005, Kodama at al., 2006). Since liver-like cells are generated by stem cells it is important to understand the role of notch signaling in those cell types.

Hepatocyte-like cells display a morphology typical for hepatocytes, i.e. they have a polygonal cell shape, a large cell diameter (about 25-50 μM), are often bi-nucleated and show a tendency to accumulate lipid granules.

AAT, CK18, HNF-3beta, Albumin and LFABP are all liver specific markers, and as such their expression is indicative of hepatocyte-like cells. However, not all of these liver specific markers are necessarily expressed in all cells of a cell population according to the present invention. Even cells that express only one, such as, e.g., only two, only three, or only four of these markers may behave similar to liver cells and thereby be useful for the above-mentioned purposes depending on what they are supposed to be used for. To study for instance metabolism by lysation of the cells, at least CYPs and GSTs are desired. To study uptake, OATPs are important and furthermore for excretion studies of e.g. BSEP or MRP-2 are desired. The more in vivo-like the study to be performed the more of those characteristics are needed. Even better is to potentially have the hepatocyte-like cells together with other liver cell types, such as macrophages and Kuppffer cells providing liver environment also with cell-cell interactions. This type of culture system could be in shape of a sandwich into which the one or more cell types are embedded and this 3D structure and the more in vivo mimicking situation could potentially further make the hepatocyte-like cells show polarity, i.e. showing one hydrophilic side towards the blood and one hydrophobic side towards the bile. For toxicity studies phase I and II metabolising enzymes are both desired due to their interaction. In addition it is desirable that the cell population is reactive to known drug inducers, whereby e.g. phase I and/or phase II metabolising enzymes are inducible.

In one embodiment of the present invention, at least about 30%, such as, e.g., at least about 40%, at least about 50%, at least about 60%, at least about 70%, at least about 80%, at least about 90%, or at least about 95% of the cells in the cell population having at least three of the above-mentioned characteristics i)-vi), exhibit at least two, such as, e.g., at least three, at least four, at least five, or all of the following characteristics Alpha-1-antitrypsin, CK18, HNF-3beta, Albumin or LFABP. In a specific embodiment, at least one of the characteristics pertaining to the drug transporter group (i.e. characteristics i)-iii)) and at least one of the characteristics pertaining to the group of drug metabolizing enzymes (i.e. characteristics iv)-vi)). Accordingly, in addition to the one or more liver specific markers, the cell population may further have at least one of said drug transporter characteristics and at least one of said drug metabolism characteristics. More specifically, the cell population according to the present invention has at least four, such as, e.g. at least five, or all six of the characteristics i)-vi).

Characteristic i) relates to the percentage of cells in the cell population comprising hepatocyte-like cells, which exhibit protein and/or gene expression of the drug transporter BSEP in the cell population according to the invention. BSEP stands for bile salt export pump and is an ATP-binding cassette (ABC) transporter that catalyses transport of molecules across extra- and intracellular membranes using the energy of ATP hydrolysis and therefore e.g. can export drugs out into the bile (often situated in vivo on what is referred to as the apical side of the hepatocyte). In one embodiment of the present invention, at least 5%, such as, e.g., at least 10%, at least 15%, at least 20%, at least 30%, at least 40%, or at least 50% of the cells in the cell population comprising hepatocyte-like cells exhibit protein and/or gene expression of BSEP.

Characteristic ii) relates to the percentage of cells in the cell population comprising hepatocyte-like cells that exhibit protein and/or gene expression of the drug transporter MRP2 in the cell population according to the invention. MRP2 stands for multi-drug resistance protein 2 and is also a member of the ABC transporter family and exports drug metabolites into the bile. In one embodiment of the present invention, at least 5%, such as, e.g., at least 10%, at least 20%, at least 30%, at least 40%, at least 50%, at least 60%, at least 70%, or at least 80% of the cells in the cell population comprising hepatocyte-like cells exhibit protein and/or gene expression of MRP2.

Characteristic iii) relates to the percentage of cells in the cell population comprising hepatocyte-like cells that exhibit protein and/or gene expression of the drug transporters OATP2 and/or OATP8 in the cell population according to the invention. OATP-2 and OATP-8 stands for organic anion transporters 2 and 8 and are both members of the OATP family, known for instance to take up toxic endogenous metabolites and xenobiotic substances from the blood. The OATPs are in vivo situated on the basolateral side of hepatocytes towards the blood. In one embodiment of the present invention at least 5% such as, e.g., at least 10%, at least 20%, at least 30%, at least 40%, at least 50%, at least 60%, at least 70%, or at least 80% of the cells in the cell population comprising hepatocyte-like cells exhibit protein and/or gene expression of OATP2 and/or OATP-8.

Characteristic iv) relates to the percentage of cells in the cell population comprising hepatocyte-like cells, that exhibit protein and/or gene expression of the drug metabolising enzyme GST A1-1 in the cell population according to the invention. Glutathione transferases (GSTs) catalyse the conjugation of xenobiotics with glutathione and are a vital part of the phase II detoxifying system. There are furthermore among 17 different human cytosolic GST subunits divided into seven classes designated e.g. A, M, P, and S. GST A1-1 is the most abundant subunit in the adult human liver in vivo. GST M1-1 is also expressed in the adult human liver, while GST P1-1 is expressed to a higher degree in fetal liver. In one embodiment of the present invention, at least 30%, such as, e.g., at least 40%, at least 50%, at least 60%, at least 70%, or at least 80% of the cells in the cell population comprising hepatocyte-like cells exhibit protein and/or gene expression of GST A1-1.

Characteristic v) relates to the percentage of cells in the cell population comprising hepatocyte-like cells, that exhibit protein and/or gene expression of at least 2 of the drug metabolising enzymes selected from the group consisting of CYP450s-1A2, -2A6, -2B6, -2C8, -2C9, -2C19-2D6, -2E1, -3A4 and -3A7 in the cell population according to the invention. CYP stands for Cytochrome P450 and is a group of enzymes that are located in the endoplasmatic reticulum of the liver. Their role is metabolism and detoxification of endogenous compounds and xenobiotics. High concentrations of these enzymes can be found in the liver and small intestine, but many CYPs are also found in other tissues. CYPs can be altered by a number of mechanisms including inhibition and induction and can vary from person to person. The CYP system is important for understanding drug metabolism, drug interactions and drug-induced hepatotoxicity.

In one embodiment of the present invention, at least 30%, such as, e.g., at least 40%, at least 50%, at least 60%, at least 70%, or at least 80% of the cells in the cell population comprising hepatocyte-like cells exhibit protein and/or gene expression of at least 2 of the following CYP450s-1A2, -2A6, -2B6, -2C8, -2C9, -2C19-2D6, -2E1, -3A4 and -3A7. Furthermore, general CYP450 enzyme activity can be shown in such cell population, and the cell population may further exhibit enzymatic activity of at least one, such as, e.g., at least two, at least three, at least four, at least five, at least six, at least seven, at least eight, at least nine, or all ten of these CYP450 proteins.

Characteristic vi) relates to the percentage of cells in the cell population comprising hepatocyte-like cells, that do not exhibit protein and/or gene expression of the Phase II enzyme GST P1-1 in the cell population according to the invention. In one embodiment of the present invention, at least 10%, such as, e.g., at least 20%, at least 30%, at least 40%, at least 50%, at least 60%, at least 70%, or at least 80% of the cells in the cell population comprising hepatocyte-like cells do not exhibit protein and/or gene expression of GST P1-1.

Furthermore, the cell population may be shown to exhibit GST enzymatic activity, which may be at least 0.01 μmol/min/mg, such as, e.g., at least 0.03 μmol/min/mg, at least 0.05 μmol/min/mg, at least 1.0 μmol/min/mg, at least 0.07 μmol/min/mg, at least 0.09 μmol/min/mg, at least 0.11 μmol/min/mg, at least 0.13 μmol/min/mg or at least 0.15 μmol/min/mg of protein in a lysate of the cell population.

In specific embodiments of the present invention, the cell composition comprises cells co-expressing CK 18 and one or more CYP drug metabolizing enzymes, such as, e.g., CYP1A2, CYP2A6, CYP2B6, CYP2D6, CYP2E1, a combination of CYP2C8, CYP2C9 and CYP2C19, or a combination of CYP3A4 and CYP3A7.

In addition to the above-mentioned characteristics, at least about 5% of the cells in the cell population according to the present invention have at least one of the following additional characteristics

A. Receptor

vii) at least 5% of the cells exhibit protein and/or gene expression of c-Met,
   B. Intercellular adhesion molecule
viii) at least 5% of the cells exhibit protein and/or gene expression of ICAM-1,
   C. Drug metabolising enzyme
ix) at least 1% of the cells exhibit protein and/or gene expression of UGT,
   D. Transcription factor
x) at least 90% of the cells exhibit no protein and/or gene expression of Oct-4.

Preferably, the cell population have at least two, such as, e.g. at least three, or all four of characteristics vii), viii), ix), or x).

Characteristic vii) relates to the level of protein and/or gene expression of the receptor c-Met in the cell population according to the invention. c-Met is the hepatocyte growth factor and/or scatter factor receptor whereby the hepatocyte-like cells are expected to respond to and have the same intracellular regulations and mechanisms (methylation) as human hepatocytes in vivo. In one embodiment of the present invention, at least 10%, such as, e.g., at least 20%, at least 30%, at least 40%, at least 50%, at least 60%, at least 70%, at least 80%, or at least 90% of the cells in the cell population comprising hepatocyte-like cells exhibit protein and/or gene expression of c-Met.

Characteristic viii) relates to the level of protein and/or gene expression of the Intercellular adhesion molecule ICAM-1 in the cell population according to the invention. ICAM-1 is an intra-cell-adhesion molecule important for cell-cell interactions in the liver. In one embodiment of the present invention, at least 10%, such as, e.g., at least 20%, at least 30%, at least 40%, at least 50%, at least 60%, at least 70%, at least 80%, or at least 90% of the cells in the cell population comprising hepatocyte-like cells exhibit protein and/or gene expression of ICAM-1.

Characteristic ix) relates to the level of protein and/or gene expression of the drug metabolising enzyme UGT in the cell population according to the invention. Uridine diphospho-glucuronosyl-transferase are like the GSTs phase II metabolising enzymes responsible for enzymatic addition of sugars to fat-soluble chemicals, both endogenous substrates as well as drugs and other xenobiotics. In mammals glucoronic acid is the main sugar used to prevent the accumulation of waste products of metabolism and fat-soluble chemicals from the environment or drugs to potential toxic levels in the body. Especially UGT2B7 is an important phase II enzyme of the adult human liver e.g. it cooperates with Cyp2C9 and Cyp3A4 to metabolise the drug diclofenac. In one embodiment of the present invention, at least 5%, such as, e.g., at least 10%, at least 20%, at least 30%, at least 40%, at least 50%, at least 60%, at least 70%, at least 80%, or at least 90% of the cells in the cell population comprising hepatocyte-like cells exhibit protein and/or gene expression of UGT. Furthermore, the cell population may be shown to exhibit UGT enzymatic activity.

Characteristic x) relates to the percentage of cells in the cell population comprising hepatocyte-like cells according to the invention, which exhibit no protein and/or gene expression of the transcription factor Oct-4. Oct-4 is a transcription factor whose expression is characteristic for the undifferentiated hBS cells, whose presence in the cell population comprising hepatocyte-like cells is undesirable. Accordingly, no or low expression of Oct-4 show they are no longer undifferentiated hBS cells which for instance in regenerative medicine is an advantage because an undifferentiated cell population could then potentially give rise to teratomas-like tissues. In one embodiment of the present invention, at least 10%, such as, e.g., at least 20%, at least 30%, at least 40%, at least 50%, at least 60%, at least 70%, at least 80%, at least 90% of the cells in the cell population comprising hepatocyte-like cells exhibit protein and/or gene expression of Oct-4.

Some of the above-mentioned characteristics i)-x) are inducible upon addition of an inducer, which may be selected from the group consisting of dexamethazone, omeprazole, alone or in combination. The inducer may also comprise Rifampicin, Dexamethasone, Desoxyphenobarbital, Ethanol, Omeprazole and Isoniazid. In this way the expression of at least one of the CYP450 proteins is inducible upon addition of an inducer. Furthermore, the expression of GST A1-1 and/or GST M1-1 proteins is inducible upon addition of an inducer. The expression of UGT protein is also inducible upon addition of an inducer.

The hepatocyte-like cells according to the present invention are capable of maintaining those of the characteristics i)-x) they exhibit during cultivation. In this context the term “maintained characteristics” is intended to mean stable protein expression over a defined culture and analysis period, which can be further shown e.g. with immuno histochemistry and measuring and comparing expression intensities. Accordingly, a cell population comprising hepatocyte-like cells according to the present invention may be cultured in vitro for at least one month, such as, e.g., for at least one week, or at least 72 hours with maintained characteristics.

In one embodiment of the present invention, the cell population comprising hepatocyte-like cells, or a subpopulation thereof, further express AFP.

For certain more organ mimicking applications maybe also other liver cell types are needed, such as Kuppffer cells and/or macrophages.

The hepatocyte-like or hepatoblast-like cells according to the present invention may prior to use be selected for certain of their respective characteristics described herein, obtain a higher yield. The cells may be purified by using antigen detection for liver marker expressed on the cell surface and subsequent FACsorting. Other alternatives for antigen based sorting is to coat culture dishes with a specific antibody and add cells from culture medium to the dish and let the cells with the right antigen bind in and the remaining cells be discarded and the bound-in cells harvested for further use. This method is sometimes referred to as immunopanning and could also be performed as negative selection, i.e. letting non-wanted cell types bind in to the antigen coated with and save the culture medium with the hepatocyte-like cells in suspension. This approach may just as well be performed on magnetic beads, so called MAC sorting or using column chromatography. Still other methods to purify cells, such as hepatocyte-like cells with specific characteristics include the use of density gradient media for cell separation based on buoyant density or size under centrifugation.

Still an alternative approach for obtaining purified populations of hepatocyte-like or hepatoblast-like cells is to perform positive or negative selection on a mixed population of hBS cell-derived cells. Both selection methods can be performed manually by cutting out pieces of cells or by addition of and exposure to an enzyme, such as collagenase IV or trypsin or a chelator, such as EDTA or even a mixture of a suitable enzyme and chelator. In one specific embodiment of the present invention, the culture dishes are washed twice with calcium/magnesium free PBS and then incubated in 0.5 mM EDTA diluted in calcium/magnesium free PBS, which results in a negative selection which gets rid of the non-hepatocyte-like or non-hepatoblast-like cell and leaves the hepatic-like cell types intact growing on mouse embryonic feeders. After additional exposure to the chelator and/or enzyme the hepatic-like cells are detached from the feeder cells and dishes to be further pooled and used in experiments.

Hepatoblast-Like Cells

In the present context, the term “hepatoblast-like cells” is intended to mean cells that express at least one of HNF3beta and AFP and have proliferative capacity. Accordingly, one embodiment of the present invention relates to a cell population derived from hBS cells, wherein at least about 10% of the cells in the cell population, express at least one of HNF3beta and AFP and have proliferative capacity and the cell population has at least two of the following four characteristics

A. Receptor

i) at least 1% of the cells exhibit protein and/or gene expression of alpha-6-integrin,
ii) at least 1% of the cells exhibit protein and/or gene expression of c-Met,
   B. Intercellular adhesion molecule
iii) at least 1% of the cells exhibit protein and/or expression of ICAM-1,
   C. Transcription factor
iv) at least 10% of the cells exhibit protein and/or expression of HNF-4 alpha.
   D. Cytokeratin
v) at least 1% of cells exhibits protein and/or expression of CK19.
vi) at least 1% of cells exhibits protein and/or expression of CK7.
   E. Epithelial cell adhesion molecule
vii) at least 1% of cells exhibits protein and/or expression of EpCAM.

It is also a prominent feature of the hepatoblast-like cells that they have a high nucleus to cytoplasm ratio, and are cuboidal in shape. Furthermore, they may have small nucleoli and granules in the cytoplasm. Preferably the cells may be between 10-30 μm in diameter.

Hepatoblast-like cells are cells of endodermal origin that have the capacity to further differentiate into hepatocyte-like cells. HNF3beta is an endodermal marker, and endoderm is along the developmental pathway towards hepatocytes. HNF3beta is also known to be expressed in the pancreas. In this context, the term “proliferative capacity” is intended to mean that the cells in the cell population are dividing.

Examples of additional endodermal markers that may be expressed by hBS cells differentiating towards hepatoblast-like and hepatocyte-like cells other than HNF3beta are, Gata4, Cdx2 (caudal-related homeobox transcription factor), Sox 17 (gene product of Sry-box containing gene 17), Pdx1 (pancreatic duodenal homeobox factor-1) and AFP, the latter of which is normally regarded as a fetal liver marker.

The hepatoblast-like cells may furthermore be proliferating, which is one indication of their progenitor status, i.e. they are not mature and fully differentiated hepatocyte-like cells. The proliferative status of cells can be shown by multiple means, such as BrdU incorporation and subsequent staining or another staining using protein markers specific for proliferative cells, such as KI67, which stains the proliferating cells in the population at the moment of fixation.

The characteristics i)-vii) for the hepatoblast-like cells are in vivo correlating markers important for hepatic development.

Characteristic i) relates to the percentage of cells in the cell population comprising hepatoblast-like cells that exhibit protein and/or gene expression of the alpha-6-integrin receptor. Alpha-6-integrin is a laminin receptor. Laminin receptors are part of the extracellular matrix in e.g. the developing liver and are expressed on many cell types, such as hepatoblasts and hepatocytes in vivo. In one embodiment of the present invention, at least 5%, such as, e.g., at least 10%, at least 20%, at least 30%, at least 40%, at least 50%, at least 60%, at least 70%, at least 80%, or at least 90% of the cells in the cell population comprising hepatoblast-like cells exhibit protein and/or gene expression of the alpha-6-integrin receptor.

Characteristic ii) relates to the percentage of cells in the cell population comprising hepatoblast-like cells that exhibit protein and/or gene expression of the c-Met receptor. In one embodiment of the present invention, at least 5%, such as, e.g., at least 10%, at least 20%, at least 30%, at least 40%, at least 50%, at least 60%, at least 70%, at least 80%, or at least 90% of the cells in the cell population comprising hepatoblast-like cells exhibit protein and/or gene expression of the c-Met receptor.

Characteristic iii) relates to the percentage of cells in the cell population comprising hepatoblast-like cells that exhibit protein and/or gene expression of the intercellular adhesion molecule ICAM-1. In one embodiment of the present invention, at least 5%, such as, e.g., at least 10%, at least 20%, at least 30%, at least 40%, at least 50%, at least 60%, at least 70%, at least 80%, or at least 90% of the cells in the cell population comprising hepatoblast-like cells exhibit protein and/or gene expression of the intercellular adhesion molecule ICAM-1.

Characteristic iv) relates to the percentage of cells in the cell population comprising hepatoblast-like cells, that exhibit protein and/or gene expression of the transcription factor HNF-4 alpha. This transcription factor is specifically expressed in endodermal cell types and is therefore indicative of hepatoblast-like cells. In one embodiment of the present invention, at least 5%, such as, e.g., at least 10%, at least 20%, at least 30%, at least 40%, at least 50%, at least 60%, at least 70%, at least 80%, or at least 90% of the cells in the cell population comprising hepatoblast-like cells exhibit protein and/or gene expression of HNF-4 alpha.

Characteristic v) relates to the percentage of cells in the cell population comprising hepatoblast-like cells that exhibit protein and/or gene expression of the cytokeratin 19. This cytokeratin is specifically expressed in hepatic stem cells and hepatoblasts but not in hepatocytes and is therefore indicative of hepatoblast-like cells. In one embodiment of the present invention, at least 5%, such as, e.g., at least 10%, at least 20%, at least 30%, at least 40%, at least 50%, at least 60%, at least 70%, at least 80%, or at least 90% of the cells in the cell population comprising hepatoblast-like cells exhibit protein and/or gene expression of cytokeratin 19.

Characteristic vi) relates to the percentage of cells in the cell population comprising hepatoblast-like cells that exhibit protein and/or gene expression of the cytokeratin 7. This cytokeratin is specifically expressed in hepatic stem cells and hepatoblasts but not in hepatocytes and is therefore indicative of hepatoblast-like cells. In one embodiment of the present invention, at least 5%, such as, e.g., at least 10%, at least 20%, at least 30%, at least 40%, at least 50%, at least 60%, at least 70%, at least 80%, or at least 90% of the cells in the cell population comprising hepatoblast-like cells exhibit protein and/or gene expression of cytokeratin 7.

Characteristic vii) relates to the percentage of cells in the cell population comprising hepatoblast-like cells that exhibit protein and/or gene expression of the epithelial cell adhesion molecule. This epithelial cell adhesion molecule is specifically expressed in hepatic progenitors but not hepatocytes and is therefore indicative of hepatoblast-like cells. In one embodiment of the present invention, at least 5%, such as, e.g., at least 10%, at least 20%, at least 30%, at least 40%, at least 50%, at least 60%, at least 70%, at least 80%, or at least 90% of the cells in the cell population comprising hepatoblast-like cells exhibit protein and/or gene expression of epithelial cell adhesion molecule.

In one embodiment of the present invention, at least about 15%, such as, e.g., at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the cells in the cell population having at least two of the above-mentioned characteristics i)-vii), express at least on of HNF3beta and AFP and have proliferative capacity. Furthermore, the cell population comprising hepatoblast-like cells may have at least three or all four of the characteristics i)-vii).

In another embodiment of the present invention, the cell population comprising hepatoblast-like cells, further has at least one of the following characteristics

F. Drug transporters:

viii) at least 1% of the cells exhibit protein and/or gene expression of BSEP,
ix) at least 1% of the cells exhibit protein and/or gene expression of MRP2.

BSEP and MRP2 may be important for carrying out drug transport processes already during development towards hepatocyte-like cells because metabolism, detoxification and excretion and may also be needed during this developmental phase.

Further Use Aspects

Due to the expression of drug transports and drug metabolizing enzymes, both the hepatocyte-like cells and the hepatoblast-like cells of the present invention are well suited for use in a medicinal product. Accordingly, a cell population described in this invention can be used for the manufacture of medicinal products for the prevention and/or treatment of pathologies and/or diseases caused by tissue degeneration, such as, e.g., the degeneration of liver tissue, liver disorders, such as, e.g., liver disorders selected from the group consisting of autoimmune disorders including primary biliary cirrhosis; metabolic disorders including dyslipidemia; liver disorders caused by e.g. alcohol abuse; diseases caused by viruses such as, e.g., hepatitis B, hepatitis C, and hepatitis A; liver necrosis caused by acute toxic reactions to e.g. pharmaceutical drugs; and tumor removal in patients suffering from e.g. hepatocellular carcinoma, and metabolic pathologies and/or diseases.

Furthermore, the hepatocyte-like cells and hepatoblast-like cells according to the present invention are suitably used for screening purposes. For example the cells may be used in a method for screening a compound for hepatocellular toxicity, comprising exposing cells from a cell population according to the present invention to the compound, and determine whether the compound is toxic to the cell. The cells may also be used in a method for screening a compound for its ability to modulate hepatocellular function, comprising exposing cells from a cell population according to the present invention to the compound, determining any phenotypic or metabolic changes in the cells that result from contact with the compound, and correlating the change with an ability to modulate hepatocellular function.

For use in regenerative medicine the hBS cells must have been derived from xeno-free hBS cells (see example 1) and furthermore during differentiation, dissociation and potential subculture never been exposed to non-human animal derived components neither directly nor indirectly. This can be achieved by using exclusively human derived components such as recombinant culture media and additives.

Method for Preparation

The cell populations according to the present invention are obtained without the use of differentiating agents, which is commonly used by others. Differentiating agents have the drawback of being toxic to the cells, which leads to low yields of the differentiated cells obtained by such methods and furthermore may affect the quality of these obtained cells. The present inventors have identified cultivation conditions that allow differentiation of hBS cells into hepatocyte-like cells and/or hepatoblast-like cells without use of differentiating agents. The methods for preparation of hepatocyte-like cells and hepatoblast-like cells according to the present invention thereby provide for improved quality and improved yields of cells. Furthermore, the obtained cells have the characteristics described herein, which characteristics render these cells particularly suitable for the applications mentioned elsewhere herein.

In one embodiment of the invention, differentiation of hBS cells to hepatocyte-like cells in 96-well plates are successfully performed. At least 50%, 60%, 70%, 80% 90% or 100% of the 96-wells are successful in differentiating hBS cells into hepatocyte-like cells. The hBS cells will differentiated to hepatocyte-like cells at according to the protocols of the invention e.g. at day 20, 25, 30 or 35.

The method according to the present invention furthermore is less labour-intensive over known methods. No expensive factors are needed as additives to the culture medium, other than bFGF, which is added in low amounts and less frequently than previously reported, which together make the method cheaper than known methods.

The method relies on intrinsic factors, excreted from the cells and not on any potential additives of more or less toxic characteristics, i.e. a milder, more physiologically relevant environment. Thus, the method relies on rarely occurring medium replacement and partly medium replacement. Toxic substances are only used for confirmation of inducibility of certain inducible enzymes.

In addition the method relies on mEF cells being crucial for the differentiation towards hepatocyte-like cells. The concentration of the mEF cells on which the hBS cells differentiate may range from between 20.000 cells/cm² to 200.000 cells/cm², such as from about 30.000 to 100.000 cells/cm², such as from 40.000 to 70.000 cells/cm², such as 52.000 cells/cm².

One other factor important for the differentiation method towards hepatocyte-like cells is the presence of bFGF, which is added to the culture medium prior to the differentiation.

The starting material for the present invention is suitably pluripotent undifferentiated hBS cells, such as undifferentiated hBS cell lines. Such material can be obtained from Cellartis AB and is also available through the NIH stem cell registry http://stemcells.nih.gov/research/registry/. Cellartis AB has two hBS cell lines (SA001 and SA002) and one subclone of SA002 (SA002.5) available through the NIH. Those hBS cell lines have been frequently used in the present invention.

Characteristics of the hBS cells recommended as starting material are the following: positive for alkaline phosphatase, SSEA-3-SSEA-4, TRA 1-60, TRA 1-81, Oct-4, negative for SSEA-1, telomerase activity, and pluripotency in vitro and in vivo (the latter shown by teratomas formation in immuno-deficient mice) (FIG. 1).

Before use, the hBS cell lines used as starting material may be derived from a LOT preparation subjected to a characterization program. The LOT preparation of hBS cell lines constitutes an expansion of the hBS cells in culture and a subsequent freezing of more than 100 straws in one single passage according to a standardized method (patent pending, WO2004098285). The morphology of the hBS cell lines are monitored before and after freezing and also in consecutive passages in the subsequent culturing after thawing of cells from the LOT. The quality of the LOT freezing is verified by an examination of the thawing recovery rate, which shall show a thawing rate of 100 percent for each straw of 10 thawed. A safety test concerning microbiological safety is then performed on the cells and the media in the passage of freezing to make sure the cells are free from contamination. The characterization program performed includes a broad range of methods to validate the differentiation status of the hBS cell lines. At first a marker expression analysis of the commonly accepted markers for undifferentiated cells (SSEA-1, SSEA-3, SSEA-4, TRA-1-60, TRA-1-81, Oct-4 and ALP) is performed. The genetic stability of the cells through out passage and freezing-thawing cycles is checked through karyotyping and FISH. The telomerase activity is measured using a Telo TAGGG Telomerase PCR ELISA^{PLUS} kit. The pluripotency of the hBS cells are examined by in vitro differentiation via an embryoid body step and through in vivo differentiation by transplantation of hBS cells under the kidney capsule of immuno-deficient SCID mice.

The starting material used herein may furthermore be completely xeno-free derived whereby completely xeno-free hepatocyte-like cells may be obtained for potential use in regenerative medicine. For xeno-free derivation of hBS cells all medium and matrix components, feeder cells and other material used may not be derived from or been in contact with any non-human animal material. Suitable components for xeno-free derivation of hBS cells and furthermore xeno-free hepatocyte-like cells are xeno-free derived human fibroblasts, such as human foreskin fibroblasts, serum-free or human serum based culture medium with recombinant growth factors, differentiation factors and/or potential other additives, and either human recombinant enzymes or sterile mechanical tools for dissociation and propagation of the cells.

An alternative starting material is endodermal cells, i.e. hBS cell derived cells that have already been committed towards the endodermal lineage, such as endodermal progenitor cells. Such cells may express one or more of the following endodermal markers: HNF3beta (hepatocyte nuclear factor 3), Gata4, Cdx2 (caudal-related homeobox transcription factor), Sox 17 (gene product of Sry-box containing gene 17), and Pdx1 (pancreatic duodenal homeobox factor-1).

One embodiment of the present invention relates to a method for preparation of a population comprising hepatoblast-like cells and/or hepatocyte-like cells according to the present invention comprising the steps of

i) in vitro differentiating hBS cells or progenitors derived from hBS cells on a supporting matrix in a serum free medium for at least 5 days,

ii) changing the medium from about every 5 days to about every 25 days,

iii) isolating cells by mechanical isolation,

iv) optional dissociating the cells obtained in step iii) by treatment with an enzyme,

v) optional sorting the cells based on surface antigen expression.

The progenitors derived from hBS cells may express HNF3beta and AFP and have proliferative capacity.

The in vitro differentiation in step i) is performed for at least 10 days, such as, e.g., at least 20 days, at least 30 days or at least 40 days. The time given for the differentiation in this step is determining whether the obtained cells have the characteristics of hepatocyte-like cells or hepatoblast-like cells. Accordingly, in order to obtain hepatocyte-like cells the in vitro differentiation of hBS cells or progenitors derived from hBS cells on a supporting matrix in a serum free medium is performed from about 18 days to about 30 days, preferably 20-27 days, more preferably about 25 days, whereas only from about 5 to about 10 days, preferably 15 days, are required for obtaining hepatoblast-like cells.

The serum free medium may be selected from the group consisting of VitroHES™, VitroHES™ supplemented with bFGF and autologuous pre-conditioned VitroHES™ (already conditioned on hepatocyte-like cells). The serum free medium may further comprise bFGF, preferably in a concentration from about 4 ng/ml to about 200 ng/ml, such as, e.g., from about 4 ng/ml to about 150 ng/ml, from about 4 ng/ml to about 100 ng/ml, from about 4 ng/ml to about 50 ng/ml, or from about 4 ng/ml to about 10 ng/ml.

In one embodiment of the invention, the serum free medium is VitroHES™ comprising bFGF. The concentration of bFGF may be from about 4 ng/ml to about 200 ng/ml, such as, e.g., from about 4 ng/ml to about 150 ng/ml, from about 4 ng/ml to about 100 ng/ml, from about 4 ng/ml to about 50 ng/ml, or from about 4 ng/ml to about 10 ng/ml. The concentration of bFGF may be 4 ng/ml.

In step ii) the serum free culture medium may be changed from about every 10 days to about every 20 days, such as, e.g., about every 12-18 days, such as every 14-15 days.

The supporting matrix may comprise feeder cells, such as, e.g., human or mouse feeder cells, or it may comprise an extracellular matrix of defined or undefined composition. Alternatively, the supporting matrix may comprise a coating comprising one or more proteins, alone or in combination, coating on the inside of a plastic cell culture vessel used for cell cultivation, or it may comprise a 3D environment, such as a porous filter. In the case of using a porous filter as supporting matrix, this porous filter may have pore sizes of about 4 μm in diameter, and it may be coated with one or more proteins, alone or in combination.

The one or more proteins used for coating of vessels or filters as described in the above, may be selected from the group consisting of collagen, laminin and combinations thereof.

The mechanical dissection of the cells carried out in step iii) may be performed by cutting out the hepatoblast-like cells and/or the hepatocyte-like cells as judged by visual inspection of the morphology of the cells. Hepatocyte-like cells display a morphology typical for hepatocytes, i.e. they have a polygonal cell shape, a large cell diameter (about 25-50 μM), are often bi-nucleated and tend to accumulate lipid granules. By experience and thorough experimentation, the morphology has been correlated to the expression of liver markers such as, e.g., Alpha-1-Antitrypsin, CK18, HNF-3beta, Albumin or LFABP. The performed selection may further be verified as hepatoblast-like cells or hepatocyte-like cells by identification by immunohistochemistry. An alternative to the mechanical dissection is to dissociate the cells from the surface on which they are growing and each other by e.g. an enzyme or a chelator or a combination thereof, and after that sort the cells by e.g. FACsorting, magnetic beads, or immunopanning. The cells may then finally be seeded in suitable culture and/or analysis vessels, such as multi-well plates in more or less defined numbers to further be used for in vitro analysis.

According to the methods described herein, the cell populations according to the present invention may be obtained in the presence of feeder cells such as human or mouse feeder cells, or they may be obtained in the absence of feeder cells. In the absence of feeder cells, the cell populations according to the present invention may be obtained using an extracellular matrix of defined or undefined composition, or using plastic cell culture vessel that has been coated on the inside with one or more proteins, alone or in combination. Suitable proteins for this purpose may be selected from the group consisting of collagen, laminin and combinations thereof. Alternatively, the cell populations according to the present invention may by obtained using a 3D environment, such as a porous filter.

An additional approach to obtain hepatocyte-like cells is to perform directed differentiation of hBS cells into hepatocyte-like cells via definitive-resembling endoderm in 3D cultures stimulated by e.g. different media compositions. Different factors or components can then be added and varied in types and concentrations, for instance serum, growth factors and other stimulating factors in the media. Briefly out-lined, undifferentiated hBS cell pieces may be cut out and transferred to filter insets of for instance a 24-well plate. All cultures may then be grown in different medium compositions and subject to analysis, such as immunohistochemical analysis on different time points to find the best window in time for maximizing the yield of hepatocyte-like cells or hepatoblast-like cells.

Still an additional approach to obtain hepatocyte-like cells or hepatoblast-like cells may be to co-cultivate hBS cells or endodermal progenitor cells derived from hBS cells with for instance pieces of liver, such as human adult liver or with organ pieces or cell types of an other species, such as with letting mouse embryonic liver stimulate the differentiation towards hepatocyte-like cells as explained in Example 2 below. Co-culture in such a system may be beneficial for the formation of 3D structures, such as clusters of hepatocytes and ducts.

Moreover, induction towards a proliferative status of the hepatocyte-like and hepatoblast-like cells may be induced by culture in medium that is adjusted for hepatocytes containing e.g. growth factors.

In a particular embodiment of the present invention, the obtained cell population may be xeno-free.

One embodiment of the present invention relates to an improved method for preparation of a population comprising hepatocyte-like cells according to the present invention comprising the steps of

i) in vitro differentiating hBS cells in a media suitable for growing hBS cells, such as the VitroHES™ media, for a period of up to 10 to 30 days, preferably 13 to 27 days, e.g. until day 15 or day 23. For example, 100% of the media can be replaced with the new hepatocyte media, and subsequently changed with 50%,

ii) changing to a new medium optimised for culture of hepatocytes such as the HCM media at day 10-40, preferably at day 13-35 e.g. at day 15 or day 23. The media may contain one or more of the following components: bovine serum albumin, ascorbic acid, epidermal growth factor, transferrin, insulin, hydrocortisone and antibiotics.

The amount of the media to replace can range from 30% up to 100%. The media can be replaced either three times a week or once a week, preferably once a week.

Moreover, the method may comprise the following steps

iii) optional adding high concentration of Dexamethasone for up to 10 days, preferably 8 days

iv) optional adding Sodium Butyrate (NaB) and HGF for up to 10 days, preferably 5 days

v) isolating cells

vi) optional dissociating the cells obtained in step ii) by treatment with an enzyme,

vii) optional sorting the cells based on surface antigen expression.

All details and particulars mentioned under the general method apply mutatis mutandis to the above-mentioned specific embodiment.

Kit

Another aspect of the invention, relates to a kit comprising i) a cell population comprising hepatocyte-like cells and/or hepatoblast-like cells, ii) one or more maturation factors and/or a maturation culture medium, and iii) optionally, an instruction for use. The maturation culture medium may be selected from the group consisting of VitroHES™, VitroHES™ supplemented with bFGF and autologuous pre-conditioned VitroHES™ (already conditioned on hepatocyte-like cells).

The one or more maturation factors are selected from the group consisting of bFGF, Epithelial Growth Factor, Hepatocyte Growth Factor and Oncostatin M.

Furthermore, the kit may comprise tools for monitoring maturation.

In one embodiment, the tools for monitoring maturation comprises

i) PCR primers against at least three, such as, e.g. at least four or at least five of the genes coding for expression markers selected from the group consisting of HNF3beta, AFP, albumin, BSEP, MRP2, OATP-2, OATP-8, GST A1-1, CYP450-1A2, CYP450-2A6, CYP450-2B6, CYP450-2C8, CYP450-2C9, CYP450-2C19 CYP450-2D6, CYP450-2E1, CYP450-3A4, CYP450-3A7, GST M1-1 and UGT, and

ii) a user's manual.

In another embodiment, the tools for monitoring maturation comprises i) antibodies against at least three, such as, e.g. at least four or at least five of the expression marker antigens selected from the group consisting of HNF3beta, AFP, albumin, BSEP, MRP2, OATP-2, OATP-8, GST A1-1, CYP450-1A2, CYP450-2A6, CYP450-2B6, CYP450-2C8, CYP450-2C9, CYP450-2C19 CYP450-2D6, CYP450-2E1, CYP450-3A4, CYP450-3A7, GST M1-1 and UGT, and

ii) a user's manual.

Additional tools for mentoring the cells are PROD assay components and components for urea and/or albumin detection in the culture medium.

### REFERENCES

Schwarz, Robert. E, et al, Defined conditions for development of functional Hepatic Cells from human embryonic stem cells, STEM CELLS AND DEVELOPMENT 14:643-655 (2005).
Rambhatla, Generation of hepatocyte-like cells from human embryonic stem cells, Cell Transplant. 2003; 12(1):1-11
Heins et. al., Derivation, characterization, and differentiation of human embryonic stem cells; Stem Cells; 2004; 22(3):367-76.
WO03055992, A method for the establishment of a pluripotent blastocyst-derived stem cell line
WO 2005/097980
WO 01/81549

### FIGURE LEGENDS

The patent or application file contains at least one drawing executed in color. Copies of this patent or patent application publication with color drawing(s) will be provided by the Office upon request and payment of the necessary fee.

FIG. 1

Characteristics of the starting material, the hBS cells, i.e (A) morphology, (B) SSEA-1 (negative), (C) SSEA-3, (D) SSEA-4, (E) TRA-1-60, (F) TRA-1-81, (G) Oct-4, (H) ALP (all from hBS cell line SA002, LOT AL002) and (I) pluripotency in vivo illustrated by a hematoxylin and eosin stained teratoma section from an immuno-deficient mouse with ectodermal tissue marked-up to the upper right, endodermal tissue to the lower right, and mesodermal tissue to the left (from hBS cell line SA121).

FIG. 2

Shows hepatocyte-like cells stained positive for the liver markers (A) Albumin, and (B) CK-18, together with (C) DAPI (nuclear), and (D) phase contrast, all on SA002, passage 56, after 23 days of differentiation on mEFs.

FIG. 3

Shows hepatocyte-like cells stained positive for the liver markers (A) AAT, and (B) HNF3beta together with (C) DAPI, all on SA034, passage 137, after 32 days in differentiation on mEF.

FIG. 4

Shows hepatocyte-like cells stained positive for the liver marker (A) LFABP on SA034, passage 135, after 25 days in differentiation on mEF and weakly positive for the early liver marker (B) AFP, on SA002, passage 56, after 23 days of differentiation on mEF.

FIG. 5

Shows (A) CK18 co-expressed with (B) Cyp1A2 on SA002, passage 63, after 23 days in differentiation on mEF. Reactivity can be clearly visualized in the microscope and in color.

FIG. 6

Shows (A) CK18 co-expressed with (B) Cyp2A6 on SA002, passage 63, after 23 days in differentiation on mEF. The Cyp protein expression could also be further induced using a CYP inducer cocktail of Rifampicin, Dexamethasone, Desoxyphenobarbital, Ethanol, Omeprazole and Isoniazid (data not shown). Reactivity can be clearly visualized in the microscope and in color.

FIG. 7

Shows (A) CK18 co-expressed with (B) Cyp2B6 on SA002, passage 63, after 23 days in differentiation on mEF. Reactivity can be clearly visualized in the microscope and in color.

FIG. 8

Shows (A) CK18 co-expressed with (B) Cyp2C8/9/19 on SA002, passage 63, after 23 days in differentiation on mEF. The Cyp protein expression could also be further induced using a CYP inducer cocktail of Rifampicin, Dexamethasone, Desoxyphenobarbital, Ethanol, Omeprazole and Isoniazid (data not shown). Reactivity can be clearly visualized in the microscope and in color.

FIG. 9

Shows (A) CK18 co-expressed with (B) Cyp2D6 on SA002, passage 63, after 23 days in differentiation on mEF. The Cyp protein expression could also be further induced using a CYP inducer cocktail of Rifampicin, Dexamethasone, Desoxyphenobarbital, Ethanol, Omeprazole and Isoniazid (data not shown). Reactivity can be clearly visualized in the microscope and in color.

FIG. 10

Shows (A) CK18 co-expressed with (B) Cyp2E1 on SA002, passage 63, after 23 days in differentiation on mEF. The Cyp protein expression could also be further induced using a CYP inducer cocktail of Rifampicin, Dexamethasone, Desoxyphenobarbital, Ethanol, Omeprazole and Isoniazid (data not shown). Reactivity can be clearly visualized in the microscope and in color.

FIG. 11

Shows (A) CK18 co-expressed with (B) Cyp3A4/7 on SA002, passage 63, after 23 days in differentiation on mEF. The Cyp protein expression could also be further induced using a CYP inducer cocktail of Rifampicin, Dexamethasone, Desoxyphenobarbital, Ethanol, Omeprazole and Isoniazid (data not shown). Reactivity can be clearly visualized in the microscope and in color.

FIG. 12

Shows inducibility of Cyp 3A4/7 and Cyp 1A2 in hepatocyte-like cells visualised by Western Blot after treatment with an inducing cocktail.

Induced hBS cells (Cyp inducer cocktail of Rifampicin, Dexamethasone, Desoxyphenobarbital, Ethanol, Omeprazole and Isoniazid for 96 hours) on SA002 (LOT AL002) passages 51, 53, 54, and 55, after 23-25 days of differentiation and SA002.5 (LOT BE002.5) passages 51, 52, 54, and 55, after 23-24 days of differentiation on mEF.

Untreated hBS cells: SA002 (LOT AL002), passages 47, 52 and 56, after 19-23 days of differentiation and SA002.5 (LOT BE002.5) passages 48, 53, and 55 after 19-26 days of differentiation on mEF.

HepG2 (Cat No HB-8065, ATCC): passage 23 as negative control.

Primary keratinocytes (Cat No C-12003, Promocell) at passage 2 as negative control. Human primary hepatocytes (male and female) thawed and freshly prepared used as positive controls. Beta-actin was used as an internal loading control.

FIG. 13

Shows general Cyp activity using PROD assay. The activity is increased upon treatment with Cyp inducers (as above) here visualised by picture brightness. Untreated cells in phase contrast (A) and PROD fluorescence (B), induced cells in phase contrast (C) and PROD fluorescence (D). Technical control (without addition of PROD to the cells) in phase contrast (E) and PROD fluorescence (F). All pictures on SA002 passage 56, after 26 days of differentiation on mEF. Pictures converted from red to yellow using Adobe Photoshop to better visualize brightness in grey scale.

FIG. 14

Shows expression of GST A1-1 before (A) and after induction (B), GST M1-1 before (C) and after induction (D), and GST P1-1 before (E) and after induction (F) by adding the cocktail of Rifampicin, Dexamethasone, Desoxyphenobarbital, Ethanol, Omeprazole and Isoniazid. For GST M1-1 and GST P1-1 no clear induction can be observed but a slight induction for GST A1-1. Pictures converted from red to yellow using Adobe Photoshop to better visualize brightness in grey scale.

FIG. 15

Shows induction of GST A1-1 in hepatocyte-like cells by Western Blot after treatment with an inducing cocktail.

SA002.5 (LOT BE002.5) passages 35, 36, 43, and 46 and SA167 (LOT CE167) passages 17, 18, 25 and 28 day 20-26 after differentiation on mEF, both untreated and treated with a CYP inducer cocktail of Rifampicin, Dexamethasone, Desoxyphenobarbital, Ethanol, Omeprazole and Isoniazid for 96 hours. A GST protein preparation and human male and female freshly prepared (thawed) primary hepatocytes were used as controls.

FIG. 16

GST enzymatic activity toward CDNB in hepatocyte-like cells derived from three different hESC lines and human hepatocytes and HepG2 cells, presented as μmol substrate conjugated/min/mg of total protein (mean ±SD, n=3).

FIG. 17

Shows immunoreactivity of hepatocyte-like cells for phase II metabolising enzymes UGT1A1 and UGT1A6.

UGT1A1 (A) co-expressed with CK18 (B), and UGT1A6 (C) double-stained with CK18 (D), all on SA002 passage 59, after 24 days of differentiation on mEF. Reactivity can be clearly visualized in the microscope and in color.

FIG. 18

Shows immunoreactivity of hepatocyte-like cells for the drug transporter OATP-2/8. (A) DAPI, (B) OATP-2/8, (C) phase contrast on SA002 passage 56, after 23 days of differentiation on mEF. Pictures converted from red to yellow using Adobe Photoshop to better visualize brightness in grey scale. Reactivity can be clearly visualized in the microscope and in color.

FIG. 19

Shows immunoreactivity of hepatocyte-like cells for the drug transporter BSEP. (A) DAPI, (B) BSEP, (C) phase contrast on SA002 passage 32, after 23 days of differentiation on mEF. Pictures converted from red to yellow using Adobe Photoshop to better visualize brightness in grey scale. Reactivity can be clearly visualized in the microscope and in color.

FIG. 20

Shows immunoreactivity of hepatocyte-like cells for the drug transporter MRP-2. (A) DAPI, (B) MRP-2, (C) phase contrast on SA002 passage 33, after 22 days of differentiation on mEF. Pictures converted from red to yellow using Adobe Photoshop to better visualize brightness in grey scale. Reactivity can be clearly visualized in the microscope and in color.

FIG. 21

Shows storage of glycogen in hepatocyte-like cells from cell line SA002, LOT AL002, passage 15 at day 21 of differentiation. Glycogen is detected by the PAS staining-system as a pink staining. The cultures were either treated, (B and D), or not treated with human saliva (A and C) prior to glycogen detection.

FIG. 22

Shows CYP and GST immuno stainings on hepatocyte-like cells differentiated on Matrigel.

(A) Cyp1A2 co-expressed with (B) Cyp 2B6 and (C) CK18 co-expressed with (D) GST A1-1 both on SA002, p57, after 24 days of differentiation on Matrigel. Pictures converted from red to yellow using Adobe Photoshop to better visualize brightness in grey scale.

FIG. 23

Shows high MRP-2 expression using QPCR in hepatocyte-like cells from cell line SA002.5, LOT BE002.5, passage 2 day 21 of differentiation related to control samples of undifferentiated hBS cells (BE002.5 passage 24, day 5 (control sample 1) and BE002, passage 62, day 4 (control sample 2)) from the same total amount of cDNA. The expression levels were calculated from the CT values (threshold cycles) obtained for the three cell samples and further compared to the expression of control sample 2. MRP2 expression in hepatocyte-like cells was between 11 and 32 times higher in the undifferentiated cells.

FIG. 24

Shows induction of Cyp 1A2 expression in hepatocyte-like cells. Untreated hepatocyte-like cells (A) and corresponding phase contrast (B) and induced (C) and corresponding phase contrast (D). Pictures converted from red to yellow using Adobe Photoshop to better visualize brightness in grey scale.

FIG. 25

Shows induction of Cyp 2B6 expression in hepatocyte-like cells. Untreated hepatocyte-like cells (A) and corresponding phase contrast (B) and induced (C) and corresponding phase contrast (D). Pictures converted from red to yellow using Adobe Photoshop to better visualize brightness in grey scale.

FIG. 26

Shows AFP-positive hepatoblast-like cells formed by hBS cells co-cultured with embryonic mouse liver. Human specific nuclear antigen in red (distinctively staining the nuclei) shows that the hBS cells are giving rise to the endodermal derived AFP-positive large cluster in green (see example 2).

FIG. 27

Shows western blot analysis of Cyp1A2, 3A4/7 and 1A1 protein expression in untreated and induced hepatocyte-like cells derived from cell line SA167 (A and B), SA002 (C and D) and SA002.5 (E and F). A, C, E: untreated cells, B, D, F: cells treated with inducer cocktail, G: human hepatocytes Lot GIU 22 (for CYP1A1 Lot MYO), H: undifferentiated hESC line SA002.5, I: MEF, J: HepG2 cells and K: recombinant CYP1A1.

FIG. 28

(A) shows the reaction for the hepatic/endodermal marker HNF4α, (B) shows reaction for Ki67, a marker used for showing proportion of proliferating cells in a population, (C) shows HNF4α and Ki67 being co-localized in a number of hepatoblast-like cells, (D) shows DAPI (staining nuclei) and (E) shows morphology.

FIG. 29

Shows the results pertaining to proliferative capacity and induction thereof as a response to the culture medium. (See example 4). (A)-(C) show hepatocyte-like cells of SA002 cells cultured in VitroHES™, with (A) showing morphology, (B) showing no reaction for the KI67 marker, (i.e. no proliferation) and (C) showing reaction for the liver marker alpha-1-antitrypsin. (D)-(F) show hepatocyte-like SA002 cells cultured in Willimas E medium, with (D) showing morphology, (E) showing reaction for the KI67 marker, (proliferation) and (F) showing reaction for the liver marker alpha-1-antitrypsin.

FIG. 30

Shows EROD reaction in hepatocyte-like cells and primary hepatocytes. Left column shows EROD activity in (from top) untreated, Omeprazol+Rifampin induced, and 6-component cocktail induced hepatcyte-like cells and the right column shows the corresponding results in primary hepatocytes. The hepatocyte-like cells accordingly have a specific Cyp 1A2 reactivity, which was also detected before treatment with Cyp inducers, although then very weak. (See example 13.)

FIG. 31

Shows Cyp activities in the hBS cell derived hepatocyte-like cells compared to in HepG2. (A) and (D) show untreated hepatocyte-like cells, (B) and (E) show induced hepatocyte-like cells and (C) and (F) show HepG2.

FIG. 32

Shows PROD reaction in hepatocyte-like cells and primary hepatocytes. Left column shows PROD activity in (from top) untreated, Primidone induced, and 6-component cocktail induced hepatcyte-like cells and the right column shows the corresponding results in primary hepatocytes. The hepatocyte-like cells accordingly have a general Cyp activity also before treatment with Cyp inducers. (See example 4.)

FIG. 33

Shows rat hepatocytes (picture from Professor Ian Cotgreave) with hepatocyte canaliculi marked out (left) and hepatocyte-like cells with canaliculi resembling structures (right).

FIG. 34

Shows a flowchart for derivation of hepatocyte-like cells from hBS cells via hepatoblast-like cells.

FIG. 35

Shows expression of membrane expressed Notch2 (in green, in membranes) and nuclear staining (in blue) in cell line SA461 (passage26) after 17 days in culture. Arrows indicate bi-nucleated hepatocyte-like cells. Magnification 250×.

FIG. 36

Shows the relative gene expression levels of Cyp3A4, 3A7, 1A1, 1A2 and Cyp2A6 were measured and compared by real-time PCR techniques in induced and non-induced cultures of hepatocyte-like cells, HepG2 and human liver extracts. Measurement of the human liver extract was set to 1 and all other samples were related to the human liver reference for each cytochrome p450. The expression for all genes is normalised against either GAPDH (CYP1A1/1A2, CYP2A6) or TBP (CYP3A4/3A7).

FIG. 37

Shows the study design of the improvement of mediums for culturing hepatocyte-like cells. Study design A; 100% of the medium was replaced from VitroHES™ to HCM after 15 days and at day 23 50% of HCM was replaced with new HCM-medium. The experiment was carried out with cell line SA002. Study design B; 100% of the medium was replaced from VitroHES™ to HCM after 23 days. The experiment was carried out with cell line SA348.

FIG. 38

FIG. 38A shows the morphology of the hepatocyte-like cells cultured in VitroHES™.

FIG. 38B shows the morphology of hepatocyte-like cells cultured in HCM. Both

FIGS. 38A and 38B show cells cultured according to study design A and B, FIG. 37 and exp.1, 2, table 5.

FIG. 39

Shows the relative mRNA expression levels of HNF4-alpha, Albumin, CYP3A4 and UGT2B7 in VitroHES™ compared with HCM according to study design A and B, FIG. 37 and exp.1, 2, table 5. Data from reference protocol (VitroHES™) was set to 1 and fold increase in expression levels of the different genes by HCM-medium is presented in the graph.

FIG. 40

Shows the study design for supplementation and induction factors in medium for culture of hepatocyte-like cells. Study design C; 50 μm Dexamethasone was added after 22-24 days. The experiment was carried out with cell line SA002, SA167 and SA348. Study design D; VitroHES™ medium was replaced after 21 days to HCM-medium supplemented with HGF and Sodium butyrate (NaB) for another 5 days.

FIG. 41

Shows the morphology of the hepatocyte-like cells cultured in VitroHES™ (A) and VitroHES™ supplemented with 50 μm Dexamethasone (B) according to study design C, FIG. 40 and exp. 1, 2, 3 table 6. As well as, hepatocytes-like cells cultured in C) VitroHES™ D) HCM supplemented with HGF and Sodium Butyrate according to study design D, FIG. 40 and exp. 1, 2 table 7.

FIG. 42

A) Shows the relative mRNA expression levels of HNF4-alpha, Albumin, CYP3A4 and UGT2B7 in hepatocyte-like cells after treatment with 50 μm Dexamethasone according to study design C, FIG. 40 and exp. 1, 2, 3 table 6. B) Relative mRNA gene expression levels after treatment with Sodium Butyrate and HGF according to study design D, FIG. 40 and exp. 1, 2 table 7. Data from reference protocol (VitroHES™) was set to 1 and fold increase in expression levels of the different genes by the different treatments is presented in the graph.

FIG. 43

Shows the study design for medium replacement frequency, rare and frequent medium replacement. Both HCM and VitroHES™ was used.

FIG. 44

FIG. 44A shows the morphology of the hepatocyte-like cells after rare medium replacement. FIG. 44B shows the morphology of the hepatocyte-like cells after frequent medium replacement. Both FIGS. 44A and 44 show cells according to study design E, as in FIG. 43 and exp.1, table 8.

FIG. 45

Shows the relative mRNA gene expression levels of HNF4-alpha, Albumin, CYP3A4 and UGT2B7 after rare medium replacement compared to frequent medium replacement according to study design E, FIG. 43 and exp.1, table 8. Data from frequent medium replacement was set to 1 and fold increase in expression levels of the different genes after rare medium replacement is presented in the graph.

FIG. 46

FIG. 46 shows three (see FIG. 46A) to six (see FIG. 46B-46C) days after reseeding of 38 days old hepatocyte-like cells on Collagen I coated 96-wells. The explants of the hepatocyte-like cells were treated with either Ca and Mg-free PBS (see FIG. 46A) or collagenase (see FIGS. 46B-46D) prior to reseeding. Cultures were double stained for the hepatocyte markers CK18 (see FIGS. 46B and 46D) and HNF3beta (see FIG. 46C). The Scalebar: 100 μm (see FIG. 46A) and 50 μm (see FIGS. 46B and 46C). For experimental details see Example 15.

FIG. 47

FIG. 47 shows immunocytochemistry and morphology of hepatoblast-like cells after five days reseeding on to Collagen I coated (see FIGS. 47A-47C ad 47G-47I) and mEF-cell layer (see FIGS. 47D-47F and 47J-47L) 96-wells. Cultures were stained for the hepatoblast markers HNF4alpha (see FIGS. 47A, 47B, 47D, and 47E) and CK19 (see FIGS. 47G, 47H, 47J, and 47K). Overlay figures of the markers with Dapi for nuclear staining is shown in FIGS. 47B, 47E, 47H, and 47K. Corresponding morphological figures for each staining are seen in FIGS. 47C, 47F, 47I, and 47L respectively. Arrows in FIG. 47C indicate bi-nucleated cells. Scalebare: 25 μm; 40× objective was used. For experimental details see Example 18.

FIG. 48

Shows ICG uptake of hepatocyte-like cells in a 30 days old culture.

FIG. 49

FIG. 49 shows hepatoblasts of a 15 day old cultures. The cells are positive for CK19 (as seen in FIG. 49A), CK7 (as seen in FIG. 49B) and EpCam (as seen in FIG. 49C).

### EXAMPLES

### Example 1

### Starting Material

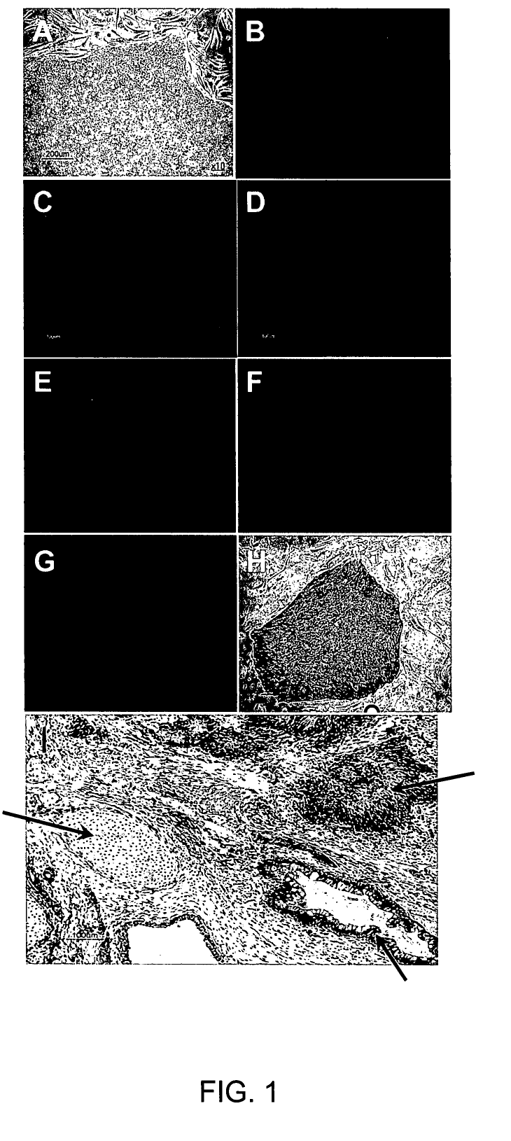

The starting material for the present invention is suitably pluripotent undifferentiated hBS cells, such as undifferentiated hBS cell lines. Such material can be obtained from Cellartis AB and is also available through the NIH stem cell registry http://stemcells.nih.gov/research/registry/. Cellartis AB has two hBS cell lines (SA001 and SA002) and one subclone of SA002 (SA002.5) available through the NIH. In addition, 20 of Cellartis cell lines are listed in the UK stem cell bank. Those hBS cell lines and in addition SA167 and SA348 from Cellartis AB have been frequently used in the present invention. Characteristics of the hBS cells recommended as starting material are the following: positive for alkaline phosphatase, SSEA-3, SSEA-4, TRA 1-60, TRA 1-81, Oct-4, negative for SSEA-1, telomerase activity, and pluripotency in vitro and in vivo (the latter shown by teratomas formation in immuno-deficient mice) (See FIG. 1.) (Methods and protocols as previously shown, Heins et al, WO03055992.)

LOT Preparation and Characterization Program

The LOT preparation of hBS cell lines constitutes an expansion of the hBS cells in culture and a subsequent freezing of more than 100 straws in one single passage according to a standardized method (patent pending, WO2004098285). The morphology of the hBS cell lines are monitored before and after freezing and also in consecutive passages in the subsequent culturing after thawing of cells from the LOT. The quality of the LOT freezing is verified by an examination of the thawing recovery rate, which shall show a thawing rate of 100% for each straw of 10 thawed, i.e. cell material can be subcultured from each individual vitrified straw upon thawing. A safety test concerning microbiological safety is then performed on the cells and the media in the passage of freezing to make sure the cells are free from contamination. The characterization program performed includes a broad range of methods to validate the differentiation status the of the hBS cell lines. At first a marker expression analysis of the commonly accepted markers for undifferentiated cells (SSEA-1, SSEA-3, SSEA-4, TRA-1-60, TRA-1-81, Oct-4 and ALP) is performed. The genetic stability of the cells through out passage and freezing-thawing cycles is checked through karyotyping and FISH. The telomerase activity is measured using a Telo TAGGG Telomerase PCR ELISA^{PLUS} kit. The pluripotency of the hBS cells are examined by in vitro differentiation via an embryoid body step and through in vivo differentiation by transplantation of hBS cells under the kidney capsule of immuno-deficient SCID mice.

The starting material used herein may furthermore be completely xeno-free derived whereby completely xeno-free hepatocyte-like cells may be obtained for potential use in regenerative medicine and so significantly decreasing the risks of graft rejection and potential transfer of non-human pathogens. For xeno-free derivation of hBS cells all medium and matrix components, feeder cells and other material used may not be derived from or been in contact with any non-human animal material. Suitable components for xeno-free derivation of hBS cells and furthermore xeno-free hepatocyte-like cells are xeno-free derived human fibroblasts, such as human foreskin fibroblasts, serum-free or human serum based culture medium with human recombinant growth factors, differentiation factors and/or potential other additives, and either human recombinant enzymes or sterile mechanical tools for dissociation and propagation of the cells.

### Example 2

### Protocols to Obtain Hepatocyte-Like Cells

Intrinsic Factor Protocol (Differentiation is Induced by Exposure to Intrinsic Factors Secreted to the Culture Medium)

a) hBS cells grown of mEF cell layers in IVF culture dishes (Falcon) are subject to differentiation under 37° C., 5% CO₂, and 95% humidity for up to 40 days to obtain hepatocyte-like cells. The culture medium used (VitroHES™ [Vitrolife AB] with 4 ng/ml of human recombinant bFGF [Invitrogen] added) is changed between every 7 and 21 days, normally every 14 days by discarding approximately 1 to 2 ml of old medium and adding 1 to 2 ml of fresh medium. After between 18 to 30 days hepatocyte-like cells are isolated from the cultures using sharp micro capillaries or the Stem Cell Tool™ (Vitrolife AB) as cutting and transfer tools and the cells are then pooled for long term storage (frozen) or immediate use, or alternatively fixed and stained directly in the culture dishes or used as living cells for e.g. Cyp activity assays. mEFs seem to provide essential signals supporting the development of hepatocyte-like cells, since the differentiation of hBS cells is drastically altered in the absence of mEFs (e.g. in hBS cell cultures on Matrigel) and much less hepatocyte-like cells can be obtained from such cultures (data not shown). This can be partly rescued by using mEF-conditioned medium for such cultures, indicating secretion of factors by mEFs into the culture medium. In accordance, we have observed that changing the medium more often than twice during the culturing period seems to be of disadvantage for obtaining hepatocyte-like cells. Another important factor for obtaining hepatocyte-like cells is supplementing the medium with bFGF.

b) hBS cells grown on Matrigel™ (Becton-Dickinson) in mEF conditioned medium supplemented with 4 ng/ml bFGF are subject to differentiation under 37° C., 5% CO₂, and 95% humidity for up to 40 days to obtain hepatocyte-like cells. The culture medium used is changed between every 7 and 21 days. After between 18 and 30 days hepatocyte-like cells are isolated from the cultures using sharp micro capillaries or the Stem Cell Tool™ as cutting and transfer tools and the cells are then pooled for long term storage (frozen) or immediate use or are fixated and used for characterization, such as immunohistochemistry.

The selection of the hepatocyte-like cells and hepatoblast-like cells in the culture dishes with differentiated cell populations is performed manually and relying mostly on morphology. The morphology has by previous experience and thorough experimentation using mainly immunohistochemistry been correlated to liver marker expression, such as those listed in Examples 3 to 7. With that experience the skilled person can actively select the hepatocyte-like cells by morphology.

To obtain hepatoblast-like cells, the same protocols are used but the incubation period is shortened down to between 10 and 20, preferred 15 days.

Filter Cultures

Undifferentiated hBS cells (BE002.5 passage 42), cultured on mEF in IVF dishes for 5 to 10 days are cut into small pieces and transferred to a filter inset (pore size: 4 μm Ø, specialized for explant cultures, Millipore) of a 24-well plate containing 400 μl media, by using glass capillary. The filter is in contact with the media surface allowing nutrition uptake of the cells and creating a moisture environment while preventing drowning the cells in media, whereby the in vivo situation is mimicked. The culture medium constitutes of 50% VitroHES™ and 50% conditioned media from undifferentiated hBS cells on mEF supplemented with 4 ng/ml bFGF. Half the media is changed every other or third day. Cells are differentiated 7, 14, 21 and 31 days, respectively before analysis. The 3D-hBS cell cultures are analyzed by immunohistochemistry. Cultures are fixed in 4% PFA following cryo-preservation in 30% sucrose and embedding in Sakura O.C.T. tissue-tek. Cryo-sections of 10 μm are analyzed morphologically and for immunoreactivity of different endodermal- and hepatocyte-like markers.

Co-Culture Protocol

Organogenesis of mouse embryonic liver can stimulate differentiation of hBS cells into hepatocyte-like cells in a 3-dimensional filter system. Liver explants of EGFP (enhanced green fluorescent protein) transgenic mouse embryos at different developmental stages were isolated and grafted next to hBS cells cultured in the filter-system. As control cultures, hBS cells alone or mouse embryonic explants other than the liver (heart and yolk sac) were grafted next to the hBS cells and cultured on filter. The co-cultures were grown in VitroHES™ supplemented with 4 ng/ml bFGF and 50% of the media was exchanged every second or third day. Day 7 and 14, co-cultures were prepared for immunohistochemical analysis. Endodermal- and hepatic markers such as HNF3beta, AFP, HNF4α, CK18, CYP3A4/7 and CYP1A2/1 were analyzed.

The amount or number of endodermal derived structures was divided into four categories, small clusters, large clusters, ducts and linings, epithelium (see definitions here below). The structures, except for the epithelium, were positive for the endodermal and early hepatic markers AFP, HNF3beta and alpha-1-antitrypsine. The number of different structures was counted for each section and culture. The total number of each structure was divided by the number of sections counted for each co-culture. This resulted in a value measuring how often the structure is occurring per section that will give an indication of the quantity of endodermal structures in a 3-dimensional hBS cell co-culture. A mean value and standard error of the mean (SEM) of each group (n=3) was calculated. The study was repeated twice.

Small cluster was defined as a gathering of cells less than or equal to five cells positive for AFP. Large cluster was defined as a gathering of cells greater than or equal to 6 cells positive for AFP. Ducts and linings formed a common category defined as mono- or multi-layered hollow structures. Epithelium was defined as an organized structure with elongated nuclei in a tight row associated with AFP-positive linings or cluster of cells. The epithelium was never positive for AFP. At day 14, all groups had developed similar amount of small clusters, while ducts and linings were only present in groups containing a mouse embryonic explant graft, thus spontaneously differentiated hBS cells were not as prone to form ducts and linings. However, large clusters were more often occurring in liver co-cultures compared to yolk sac- and heart co-cultures and spontaneously differentiated hBS cells. Structures, such as large clusters and ducts were positive for endodermal and hepatic markers such as HNF3beta, AFP, α-1-antitrypsine, Hnf4α, CYP3A4/7 and CYP1A2/1. However the clusters were negative for CK18 potentially indicating immature hepatocyte-like cells.

Altogether, the data indicates that hBS cells can differentiate more efficiently into hepatoblast-like cells or hepatocyte-like cells during direct contact with liver from E10.5 mouse embryo than alone.

TABLE 1
listing hepatic markers analysed by immunohistochemistry
after 14 days of co-culture with hBS cells with
E10.5 embryonic mouse liver.

| IHC Markers | Cluster/duct |
|---|---|
| HNF3beta | + |
| HNF4alpha | + |
| AFP | + |
| AAT | + |
| CK18 | − |
| CYP3A4/7 | + |
| CYP1A2/1 | + |

### Example 3

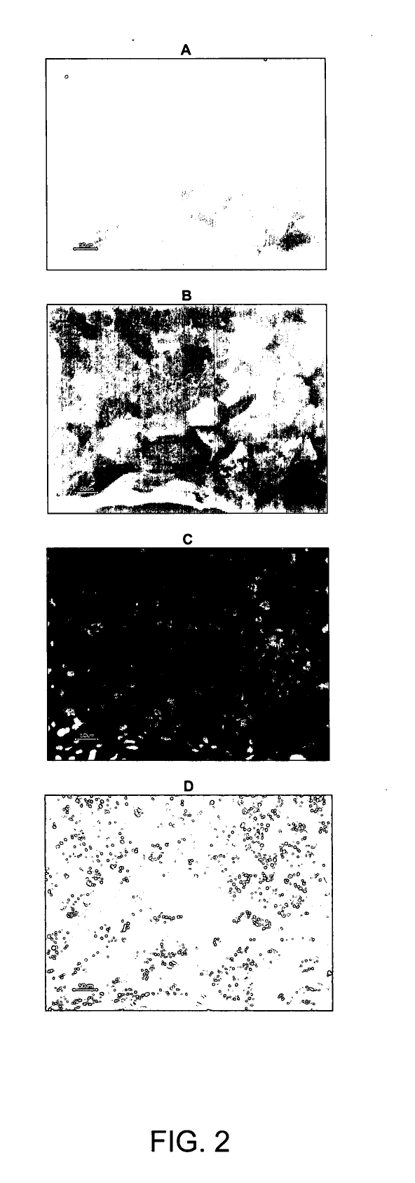

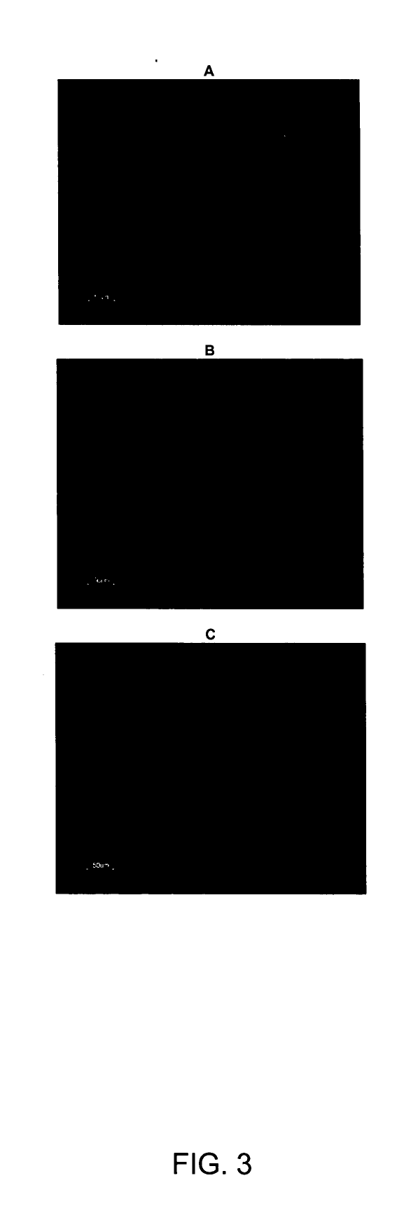

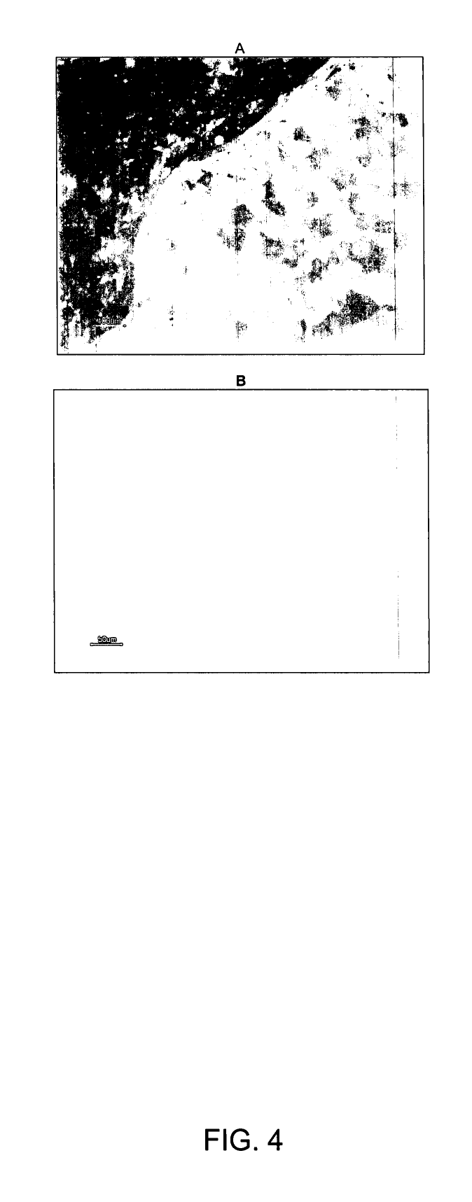

### Characterisation with Hepatic Markers (See FIGS. 2, 3 and 4)

Hepatocyte-like cells display a morphology typical for hepatocytes, i.e. they have a polygonal cell shape, a large cell diameter (about 25-50 μM), are often bi-nucleated and tend to accumulate lipid granules. Furthermore, they express several markers described for hepatic cell types, e.g. Albumin, α1-Antitrypsin, LFABP, CK18, and HNF3beta. They no longer express Oct-4, a stem cell marker used for undifferentiated cells. Some presumably less mature hepatocyte-like cells still express the fetal liver marker AFP. These cells are preferentially found inside colonies of differentiating hBS cells. DAPI (4′,6′-diamidino-2-phenylindole dihydrochloride hydrate. Sigma Aldrich) as a control to visualize cell nuclei. (For CK18 expression in hepatocyte-like cells differentiated on Matrigel™, see FIG. 22.)

For identification of proliferative hepatoblast-like progenitor cells AFP, HNF4alpha, CK19, CK7 and EpCam were used. (FIG. 49)

Used Primary Antibodies:

Albumin (rabbit) 1:500, DAKOCytomation, A0001

MT (rabbit) 1:200, DAKOCytomation, A0012

CK18 (mouse) 1:200, DAKOCytomation, M7010

LFABP (goat) 1:500, Santa Cruz, sc-16064

HNF3b (goat) 1:250, Santa Cruz, sc-6554

Oct-4 (mouse) 1:500, Santa Cruz, sc-5279

c-Met (HGF receptor, mouse) 1:100, upstate, 05-237

α6-integrin (CD49f, rat) 1:250, BD Biosciences, 555736

ICAM-1 (CD54, mouse) 1:500, BD Pharmingen, 559047

AFP (mouse) 1:200, SIGMA, A8452

HNF4alpha (rabbit) 1:400, Santa Cruz, sc-8987

CK19 (mouse) 1:200, Novocastra, NCL-CK19

CK7 (mouse) 1:200, Novocastra, NCL-L-CK7-560

EpCAM-FITC, 1:200, GeneTex, Inc. GTX28666

Used Secondary Antibodies:

donkey anti-goat-Alexa 488, 1:500, Molecular Probes, # A-11055

donkey anti mouse-Cy3, 1:1000, Jackson Immuno Research, #715-165-151

donkey anti-mouse-Cy2, 1:100, Jackson Immuno Research, #715-225-151

donkey anti-rabbit-Alexa488, 1:1000, Molecular Probes, #A-21206

donkey anti-rabbit-Alexa594, 1:1000, Molecular Probes, #A-21207

donkey anti-rat-Cy3, 1:500, Jackson Immuno Research, #712-165-153

donkey anti-rat-Cy2, 1:100, Jackson Immuno Research, #712-225-153

Immunostaining Protocol:

Albumin, MT, CK18, HNF3b, Oct-4, CK19, CK7, AFP:

15 min fixation in 4% PFA, 2×PBS wash, 30 min 5% FBS in 0.1% PBT, primary antibodies incubated in 1% FBS in 0.1% PBT overnight at 4 C, secondary antibodies in 1% FBS in 0.1% PBT for 3 hr at RT, all washes in 0.1% PBT, DAPI at 0.05 mg/ml for 5 min at RT, mounted in DAKOCytomation mounting medium.

LFABP, c-Met, α6-integrin, ICAM-1, CXCR4:

15 min fixation in 4% PFA, 2×PBS wash, 30 min 5% FBS in PBS, primary antibodies incubated in 1% FBS in PBS overnight at 4 C, secondary antibodies in 1% FBS in PBS for 3 hr at RT, all washes in PBS, DAPI at 0.05 mg/ml for 5 min at RT, mounted in DAKOCytomation mounting medium.

EpCam:

15 min fixation in 4% PFA, 2×PBS wash, 30 min 5% FBS in 0.1% PBT, FITC direct conjugated primary antibody incubated in 1% FBS in 0.1% PBT overnight at 4 C, wash in PBS, DAPI at 0.05 mg/ml for 5 min at RT, mounted in DAKOCytomation mounting medium.

### Example 4

### Characterisation

Phase I Metabolic Enzymes:

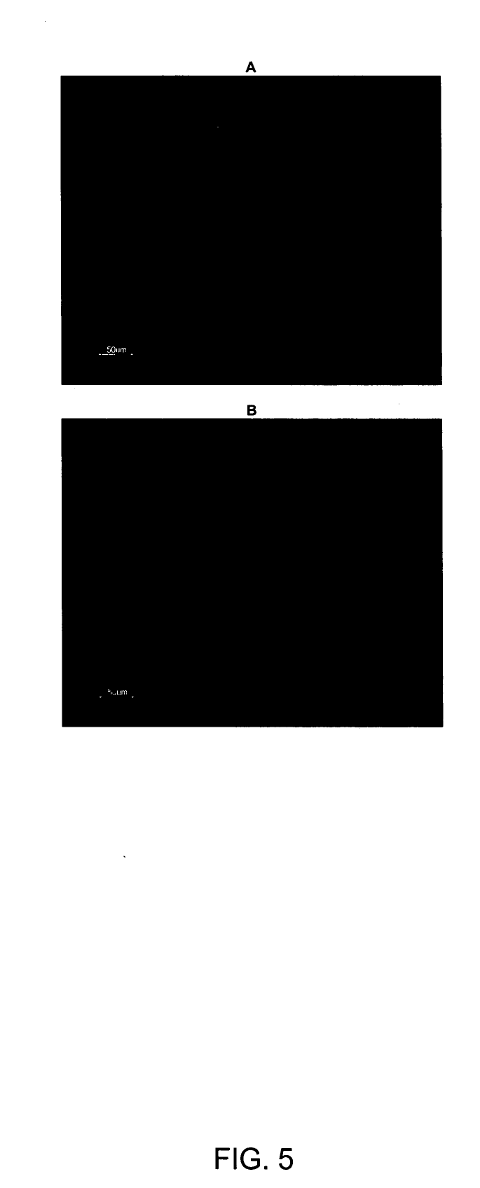

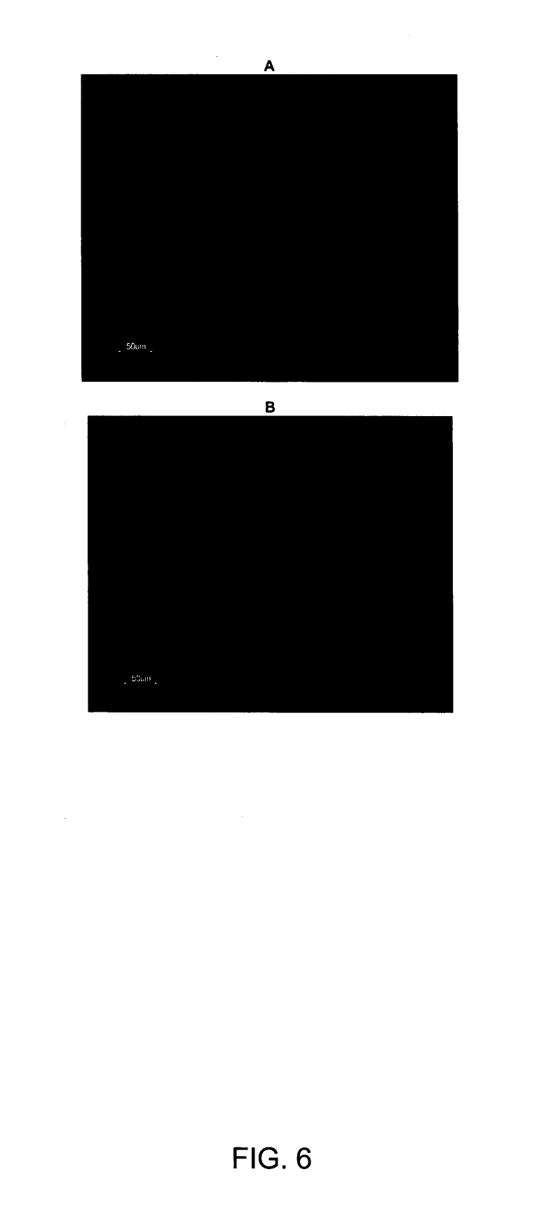

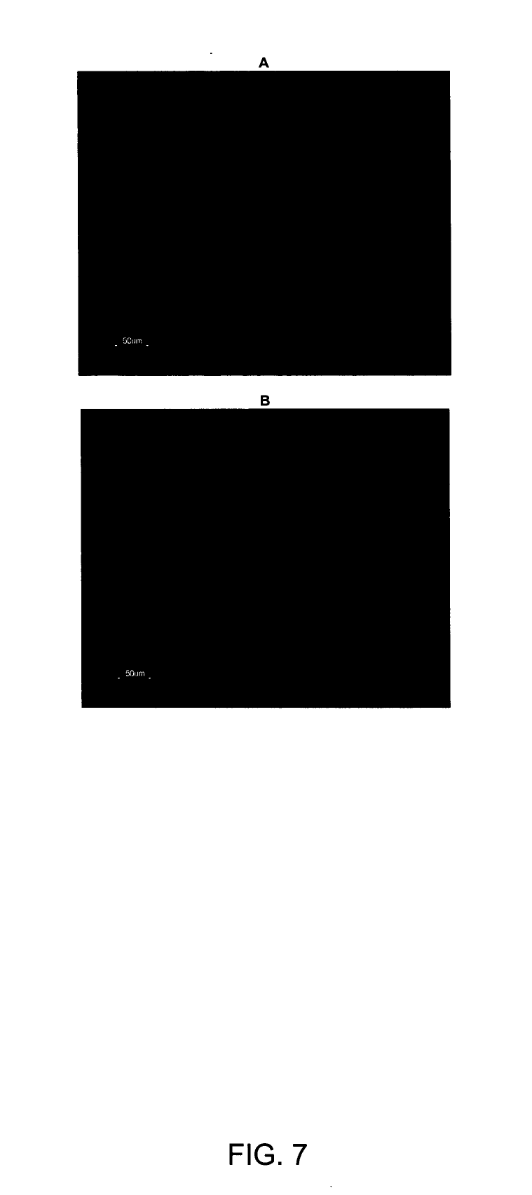

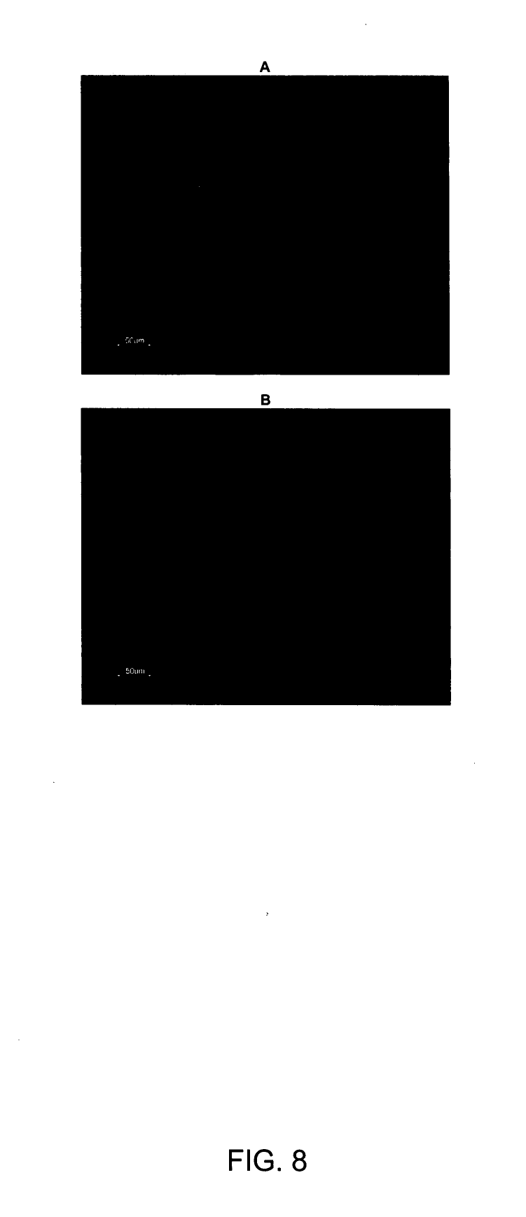

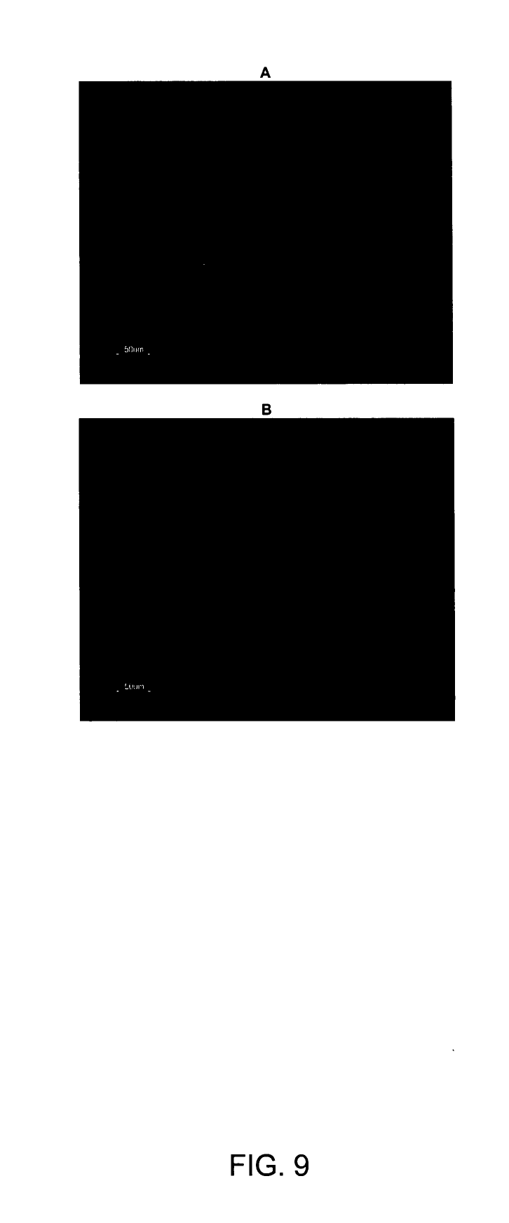

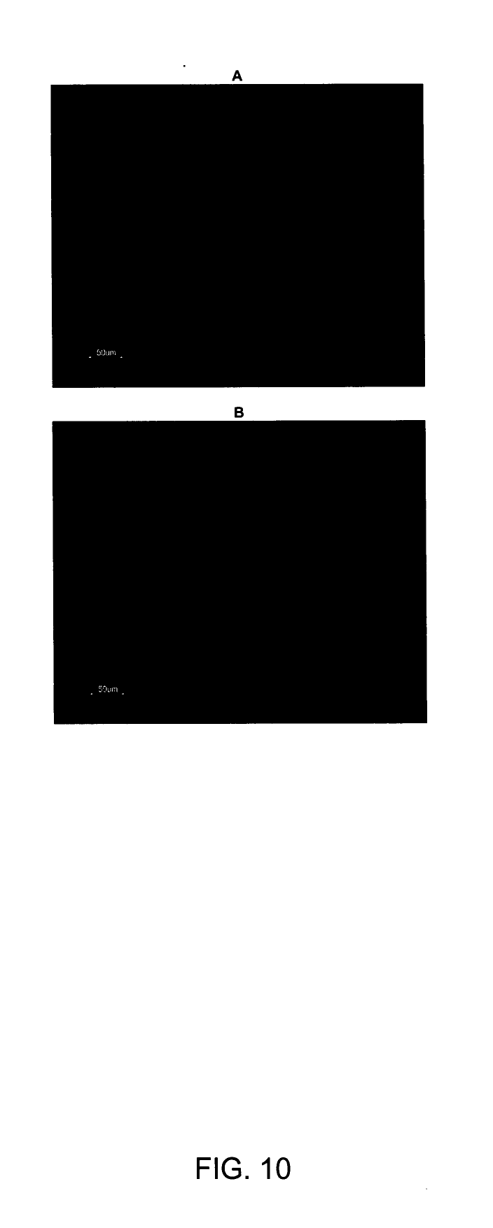

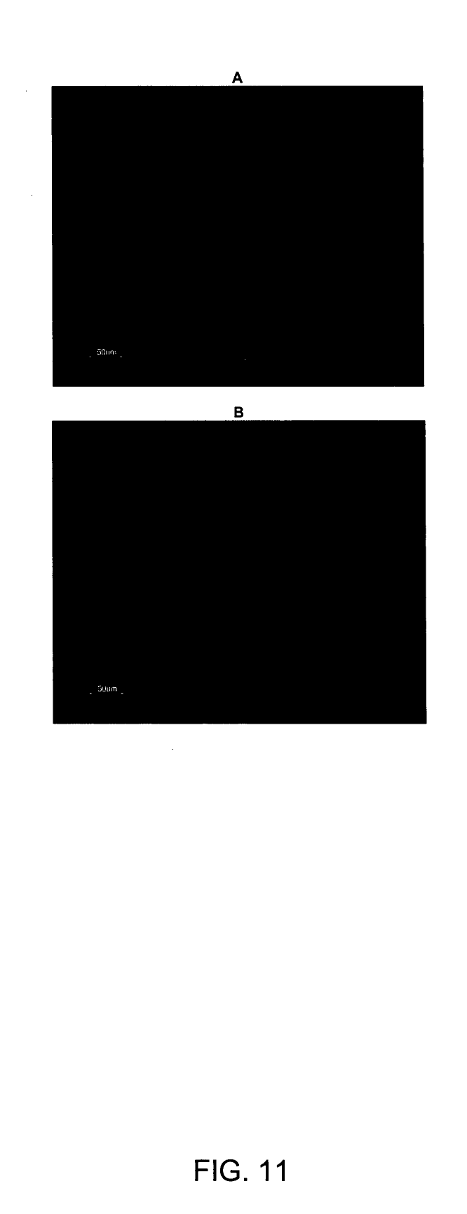

Hepatocyte-like cells display immunoreactivity for the following Cyps: 1A2, 2A6, 2B6, 2C8/9/19 (potential antibody cross reaction between the three subtypes), 2D6, 2E1, 3A4/7 (potential antibody cross reaction between the two subtypes). (For hepatocyte-like cells differentiated on mEF, see FIGS. 5-11 and for Cyp1A2 and Cyp2B6 expression in hepatocyte-like cells differentiated on Matrigel™, see FIG. 22). Inducibility of Cyp expression was seen after 96 hours of exposure to a Cyp inducing cocktail containing 25 μM Rifampin, 20 μM Desoxyphenobarbital (Primidone), 100 μM Dexamethasone, 88 mM Ethanol, 25 μM Omeprazole, and 100 μM Isoniazid. (See FIGS. 24 and 25 for induction of Cyp 1A2 and 2B6 in hepatocyte-like cells on mEF.) Cyps were also analysed immunohistochemically in HepG2, which did not give rise to any reaction (see FIG. 31).

Used Primary Antibodies:

Cyp1A2 (rabbit) 1:100, biomol, CR3130

Cyp2A6 (rabbit) 1:100, biomol, CR3260

Cyp2B6 (sheep) 1:100, biomol, CR3295

Cyp2C8/9/19 (rabbit) 1:100, biomol, CR3280

Cyp2D6 (sheep) 1:100, biomol, CR3245

Cyp2E1 (rabbit) 1:100, Chemicon, AB1252

Cyp3A4/7 (sheep) 1:100, biomol, CR3345

Cyp2E1 (rabbit) 1:1000, Oxford Biomedical Research, PA26

Cyp3A4/7 (rabbit) 1:1000, Oxford Biomedical Research, PA32

Used Secondary Antibodies:

donkey anti-goat-Alexa 488, 1:500, Molecular Probes, # A-11055

donkey anti mouse-Cy3, 1:1000, Jackson Immuno Research, #715-165-151

donkey anti-mouse-Cy2, 1:100, Jackson Immuno Research, #715-225-151

donkey anti-rabbit-Alexa488, 1:1000, Molecular Probes, #A-21206

donkey anti-rabbit-Alexa594, 1:1000, Molecular Probes, #A-21207

donkey anti-rat-Cy3, 1:500, Jackson Immuno Research, #712-165-153

donkey anti-rat-Cy2, 1:100, Jackson Immuno Research, #712-225-153

donkey anti-sheep-Alexa488, 1:1000, #A-11015

donkey anti-sheep-Alexa594, 1:1000, #A-11016

Immunostaining Protocol:

15 min fixation in 4% PFA, 2×PBS wash, 30 min 5% FBS in 0.1% PBT, primary antibodies incubated in 1% FBS in 0.1% PBT overnight at 4° C., secondary antibodies in 1% FBS in 0.1% PBT for 3 hr at RT, all washes in 0.1% PBT, DAPI at 0.05 mg/ml for 5 min at RT, mounted in DAKOCytomation mounting medium.

Western Blot:

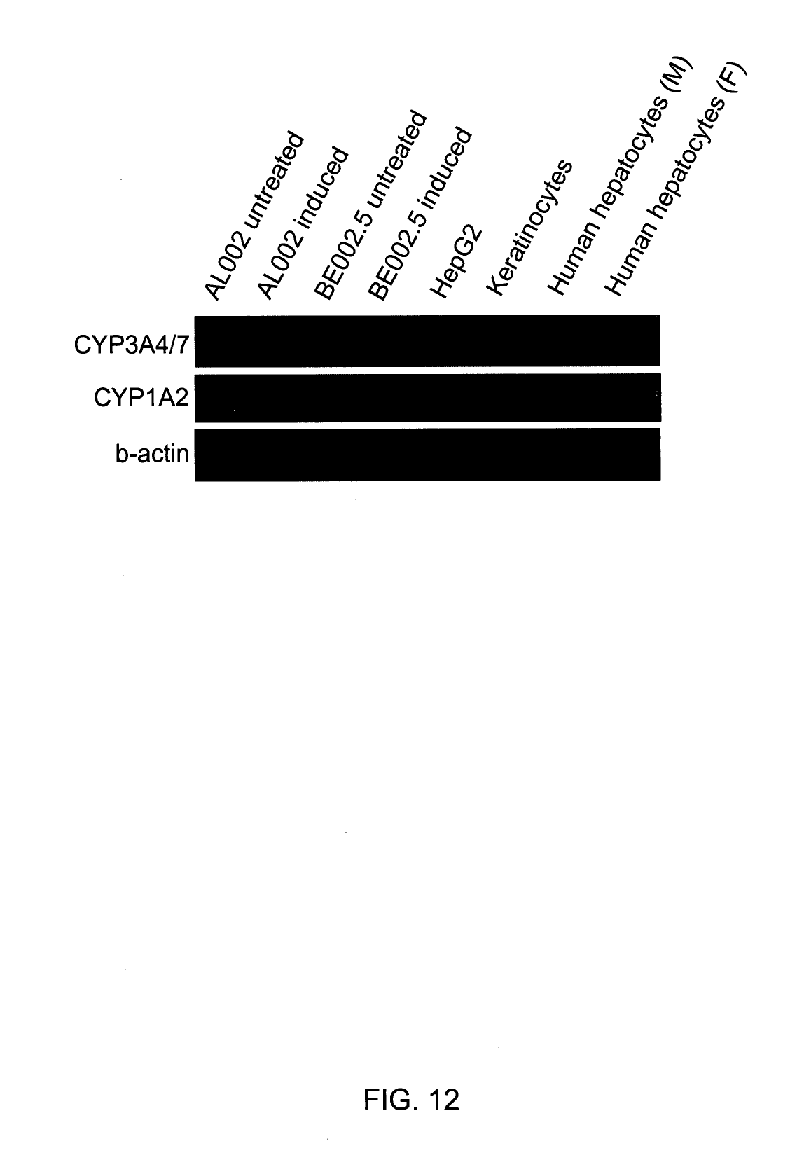

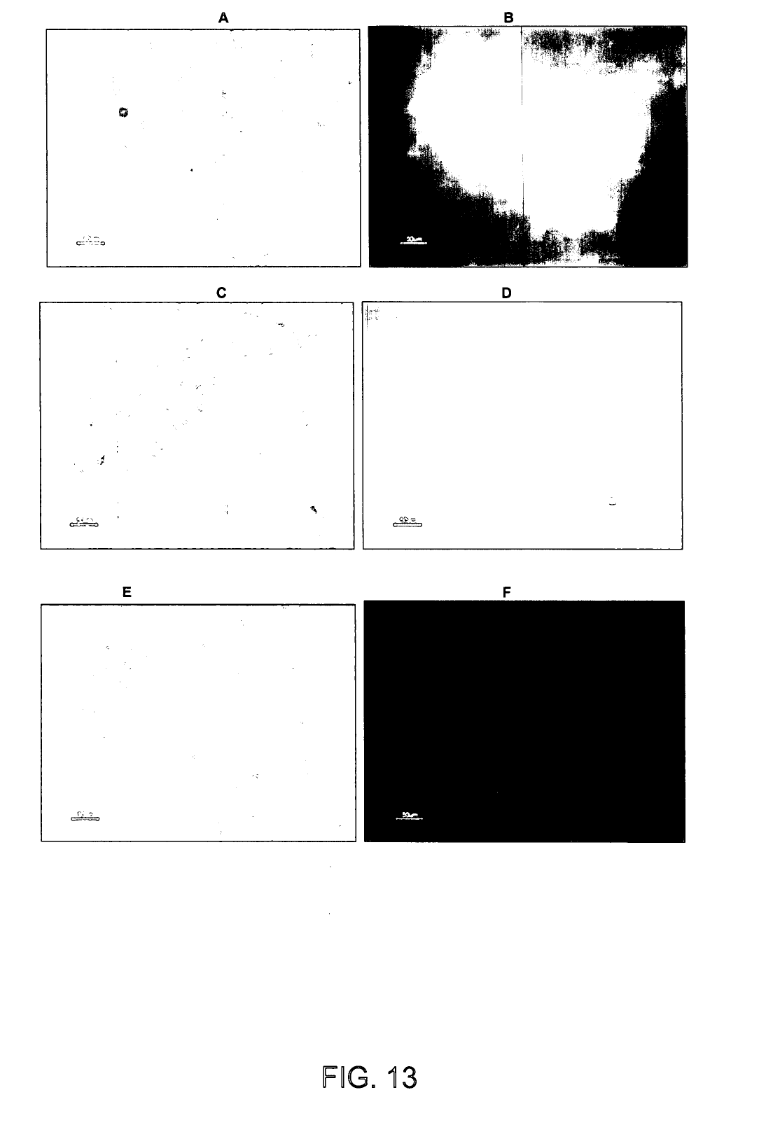

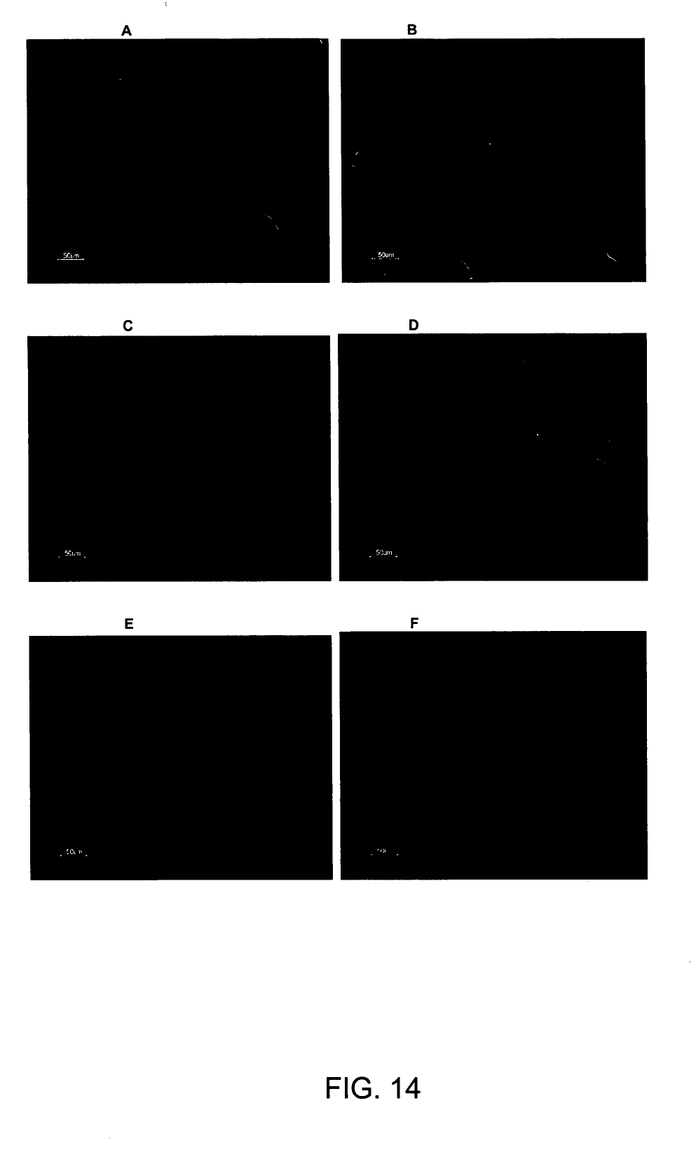

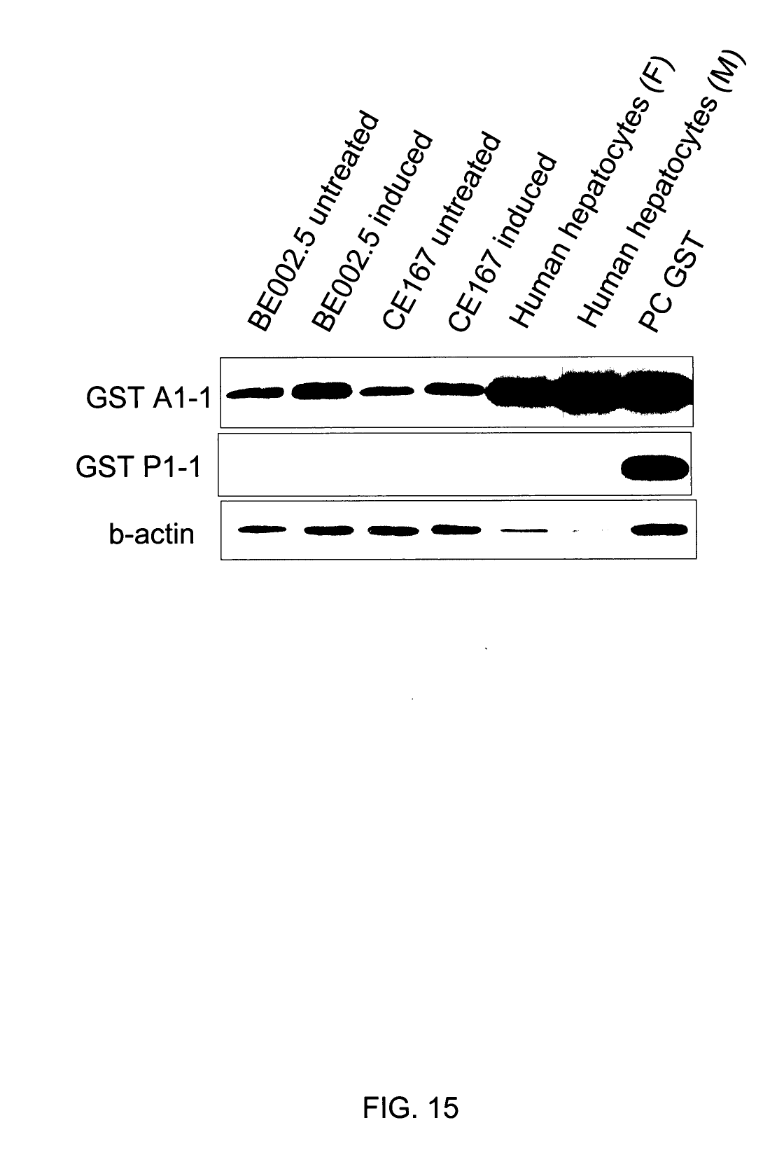

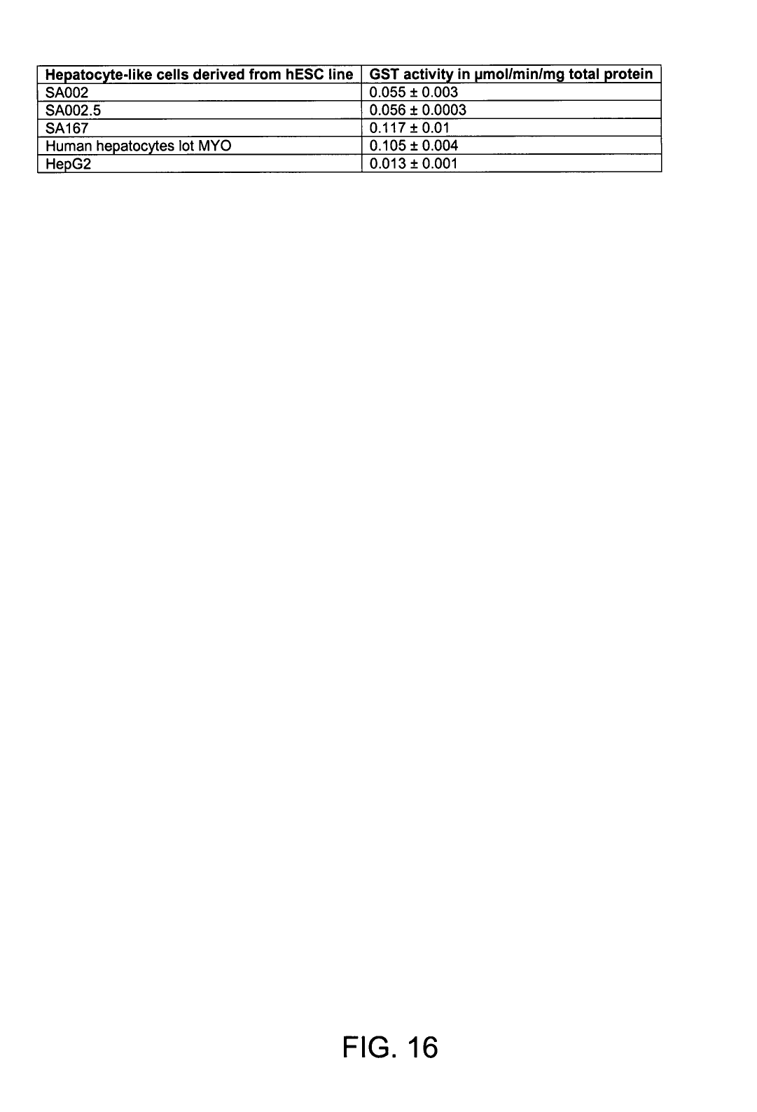

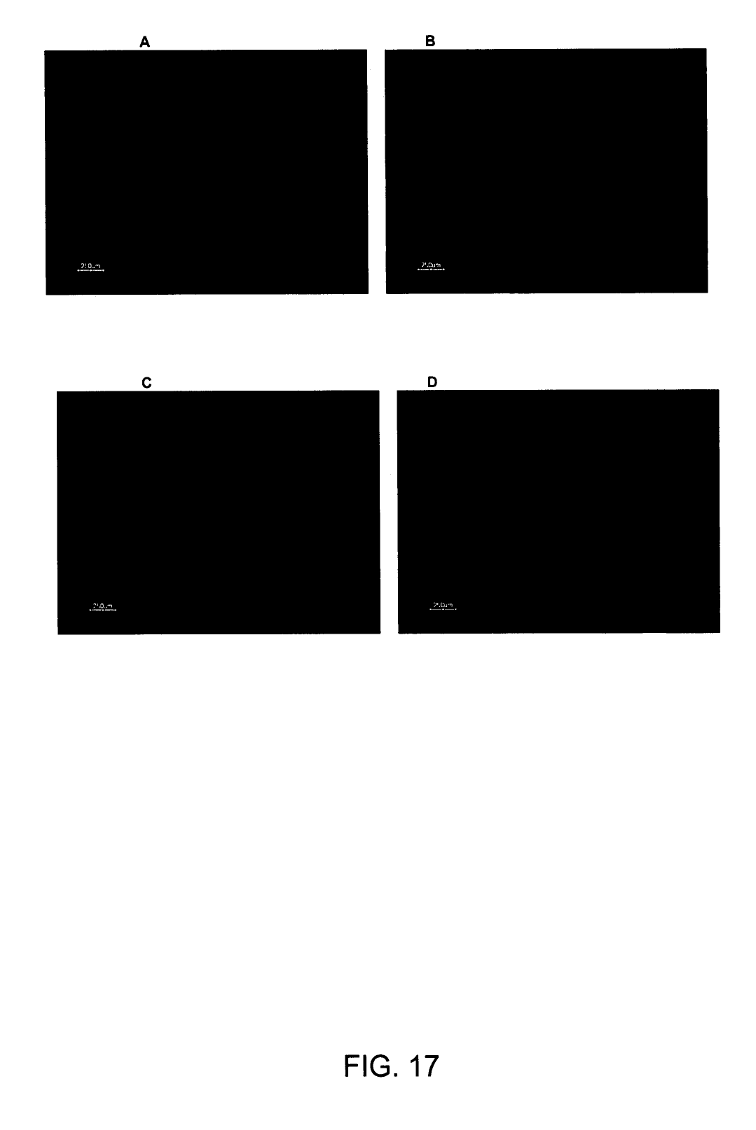

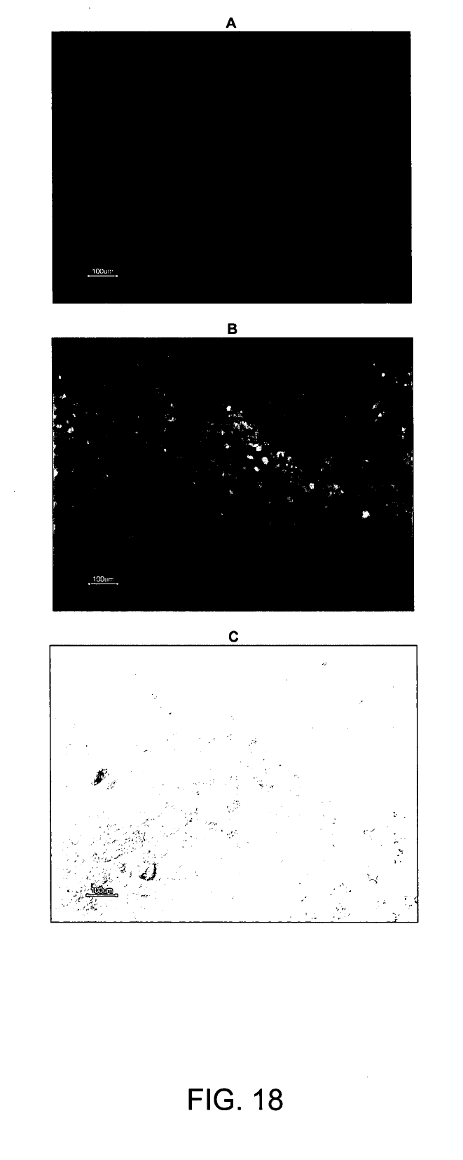

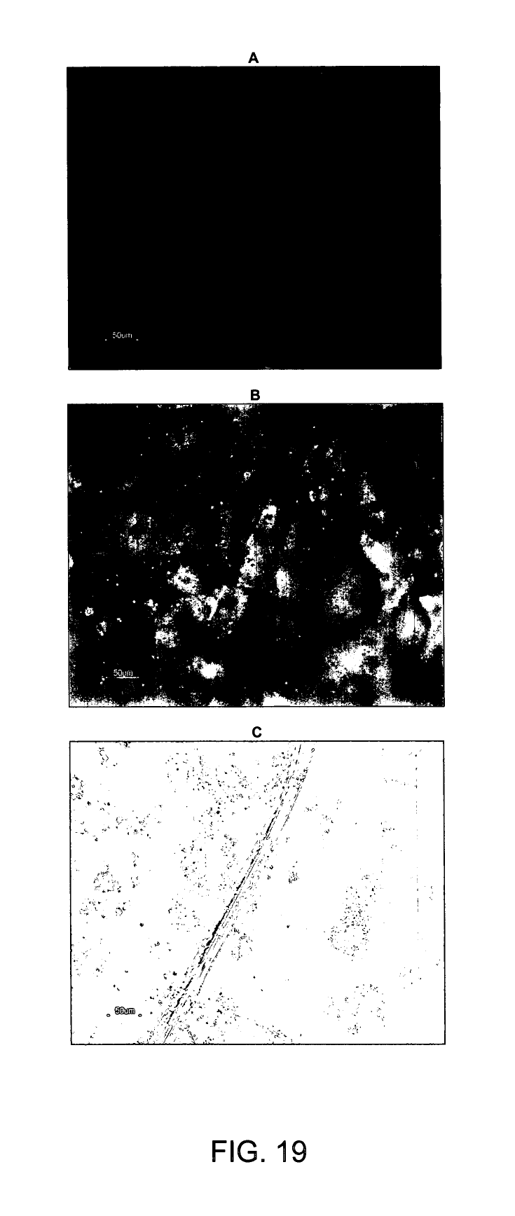

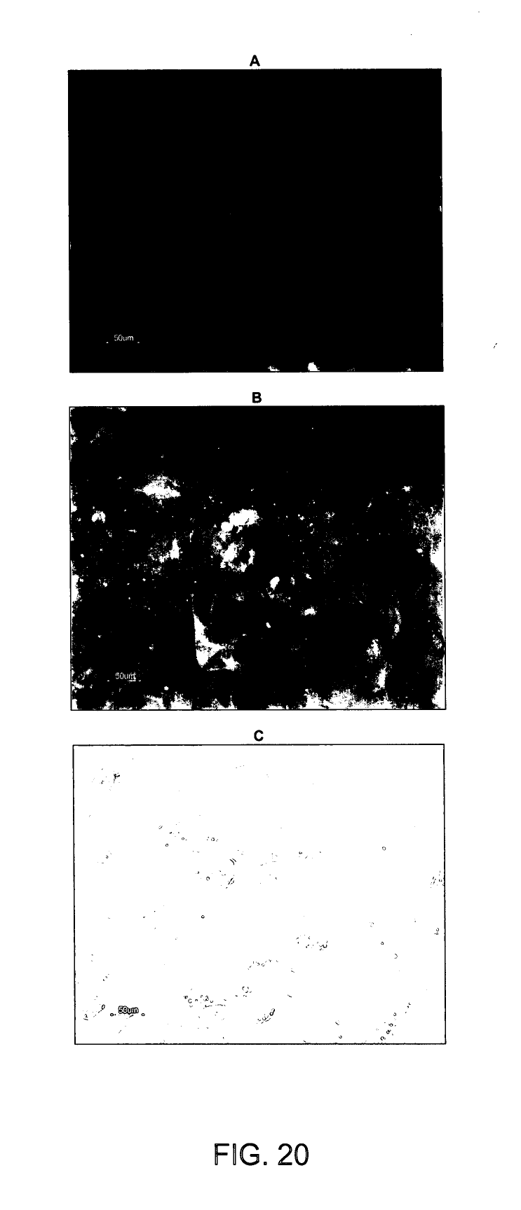

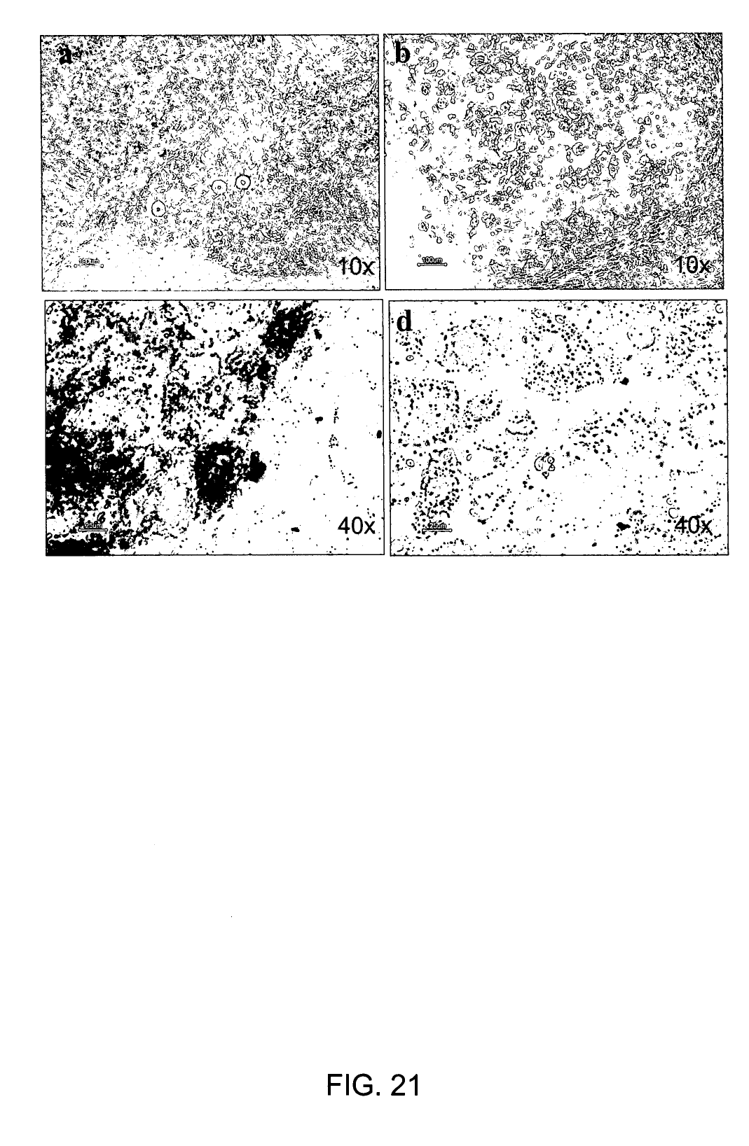

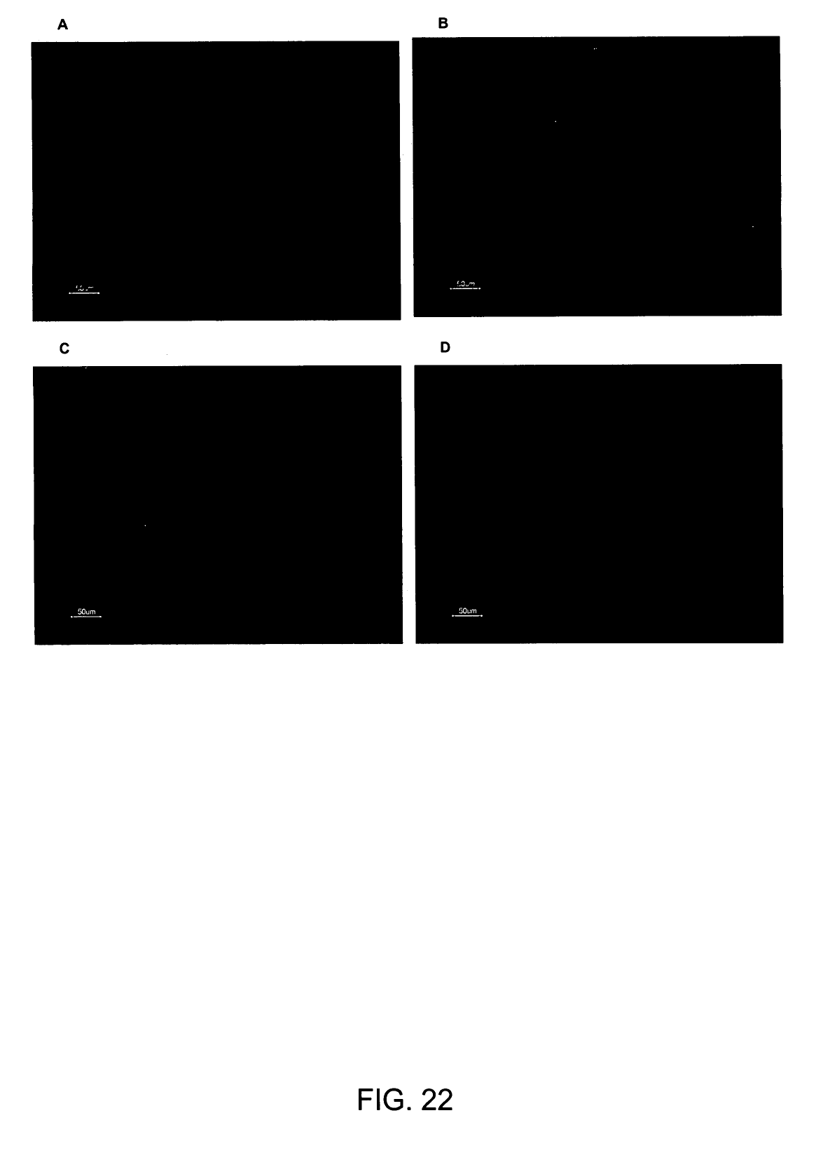

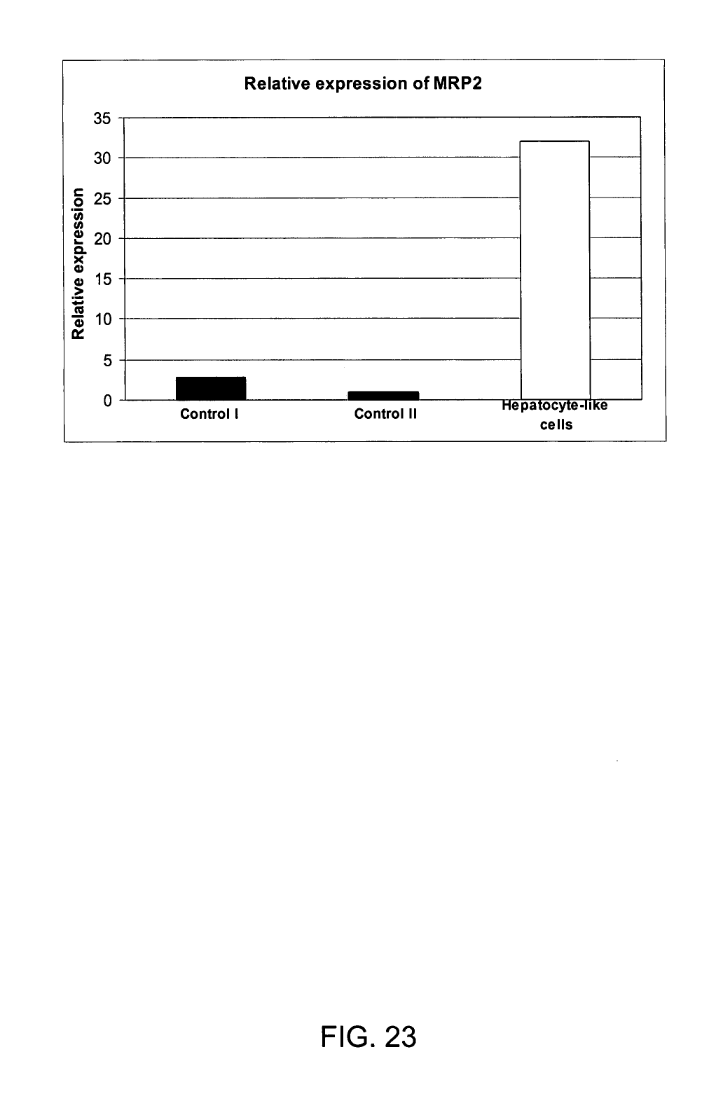

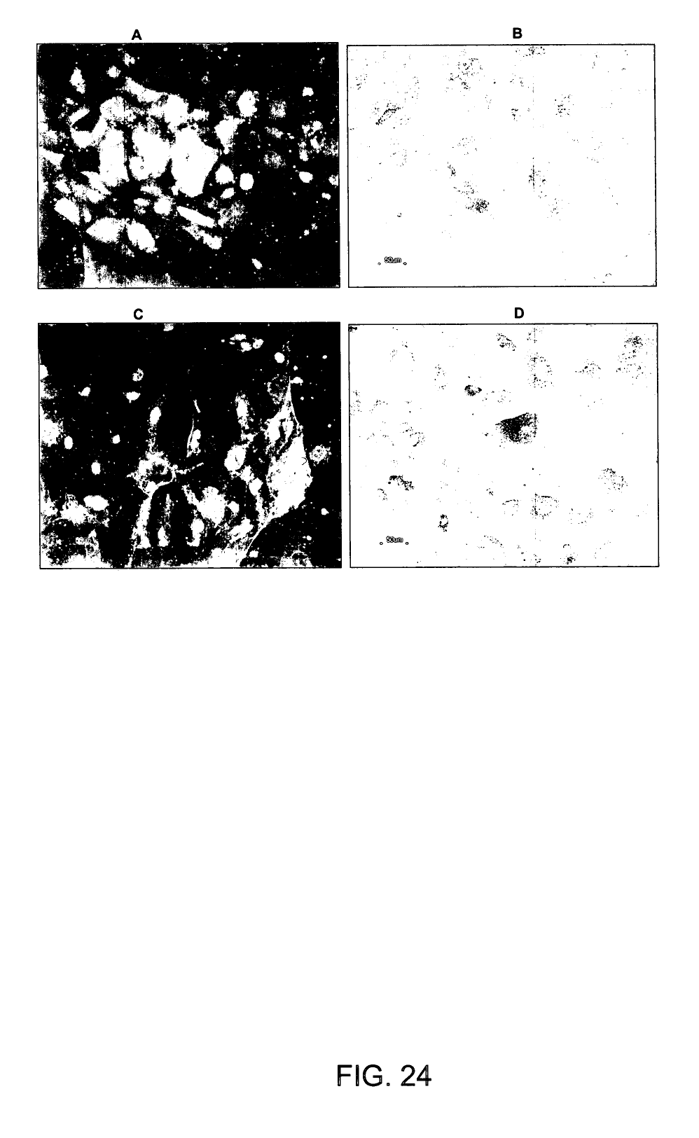

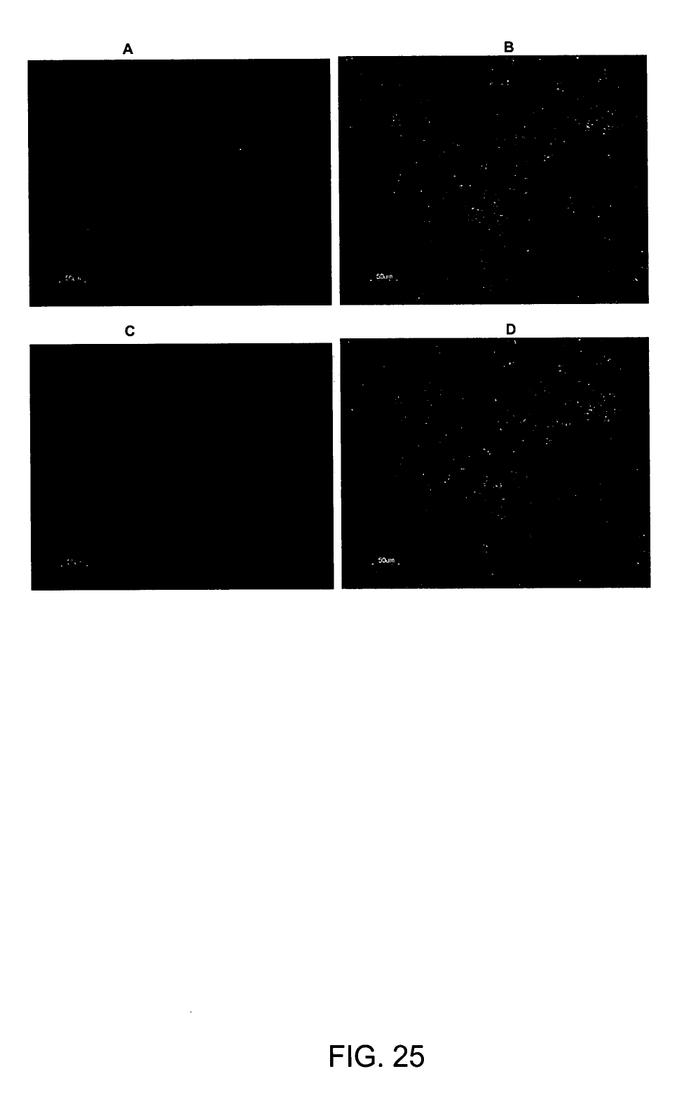

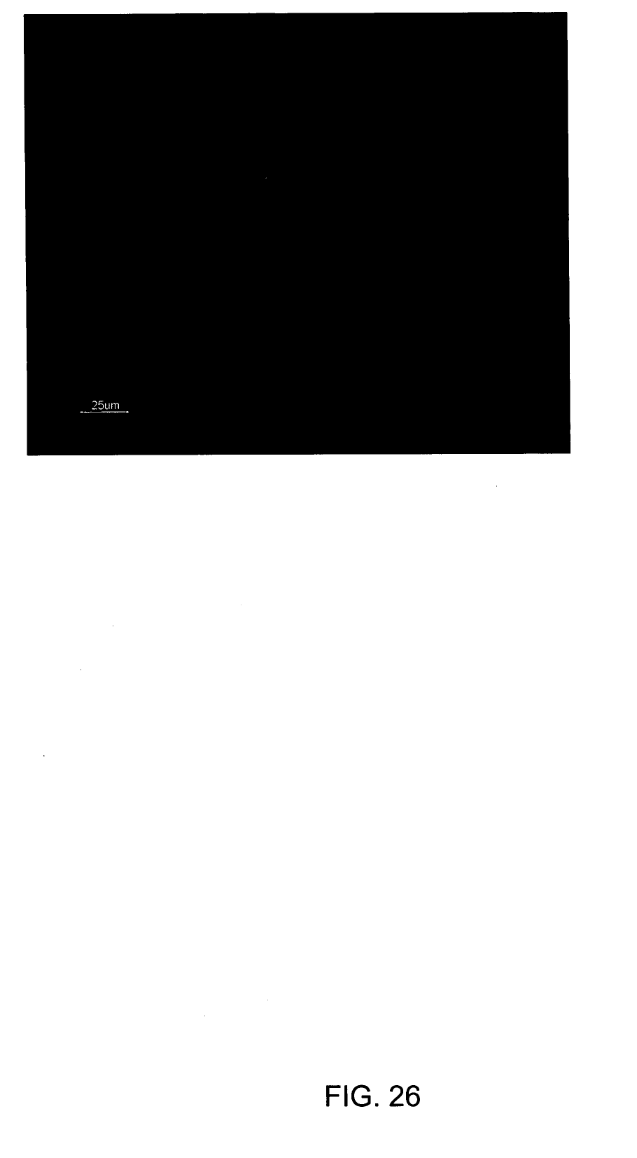

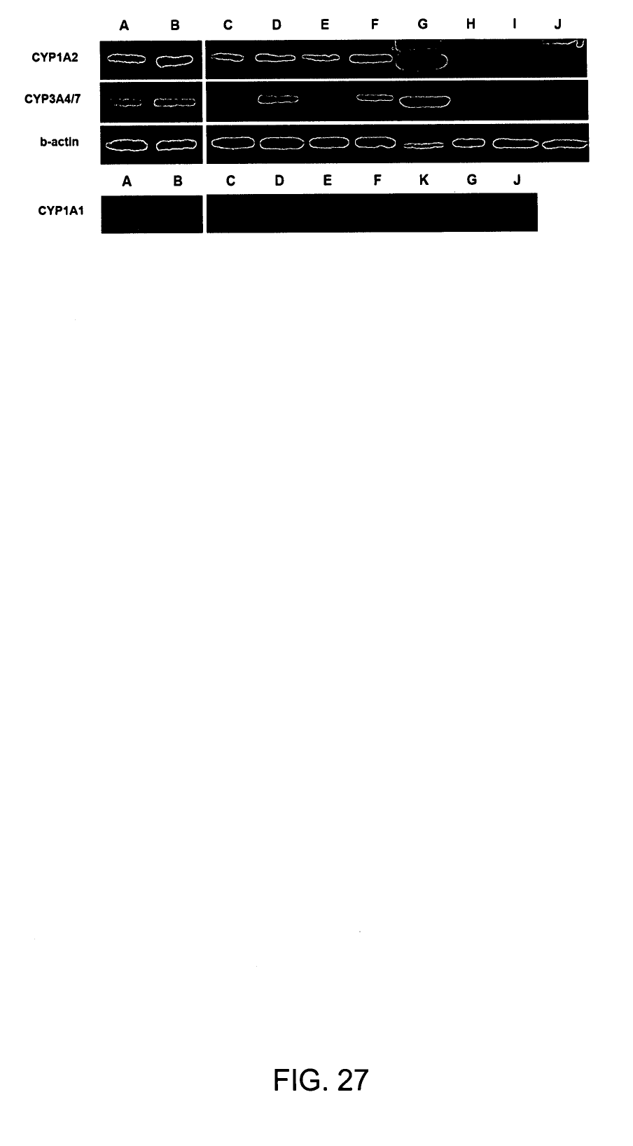

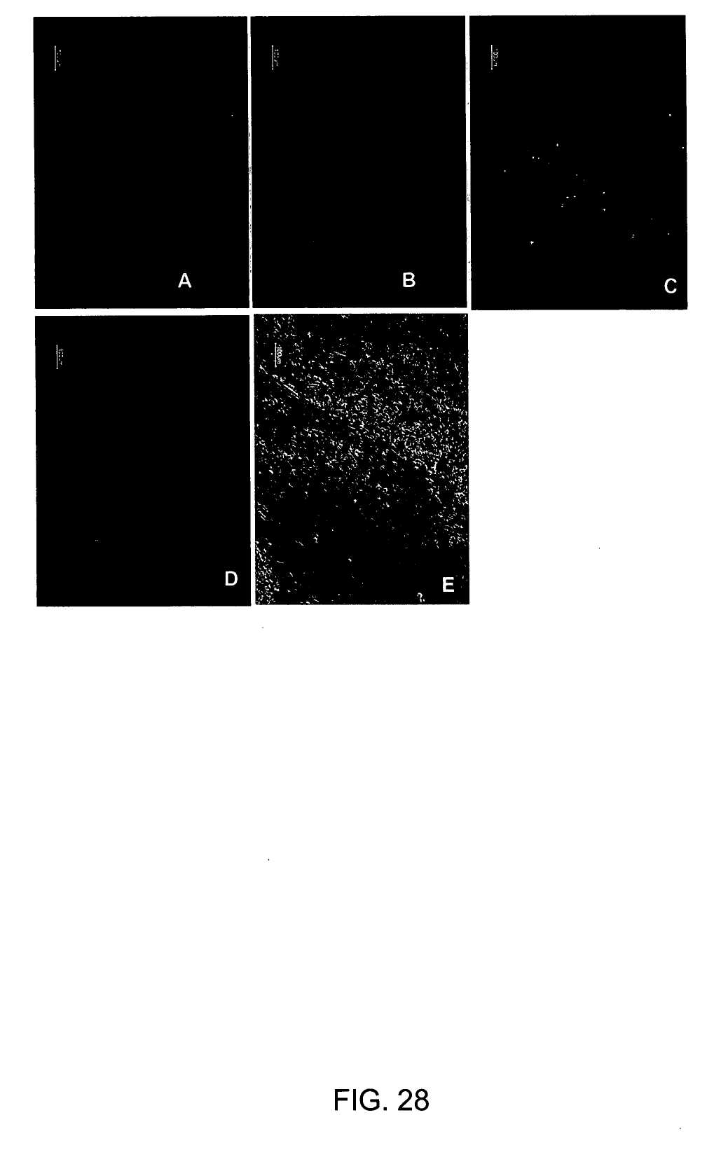

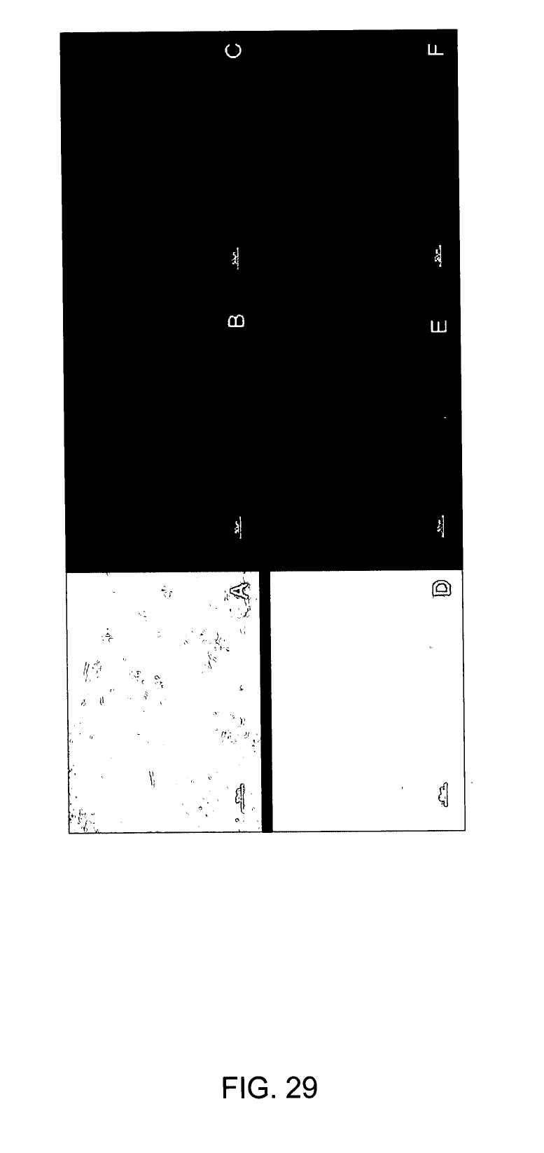

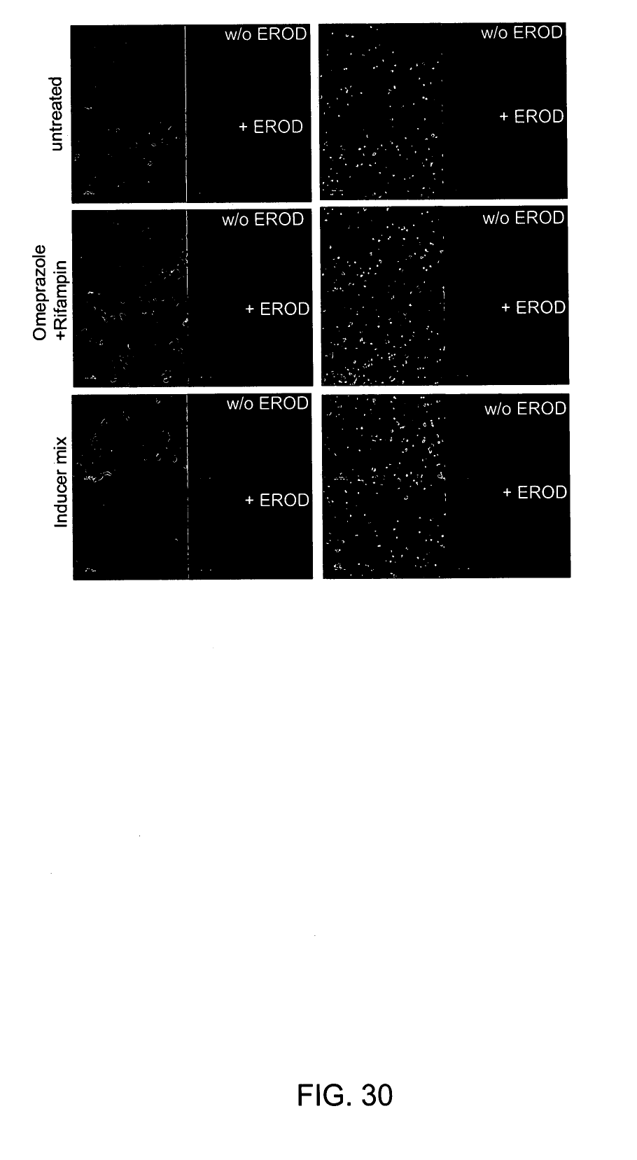

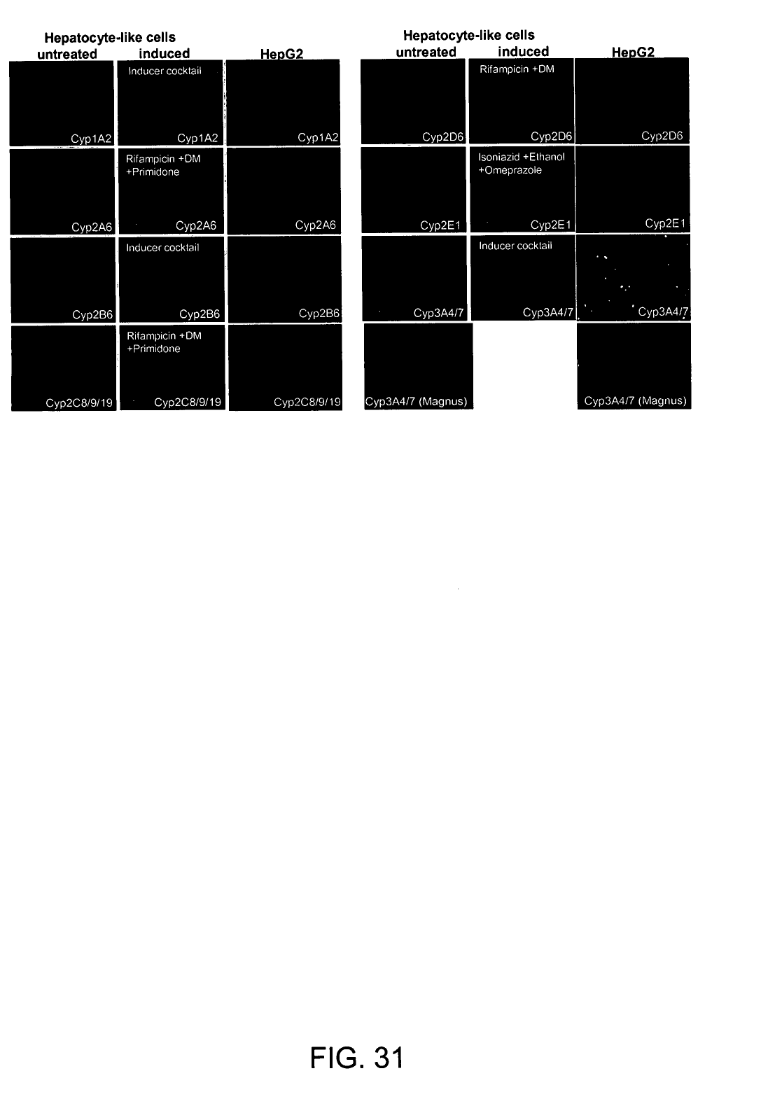

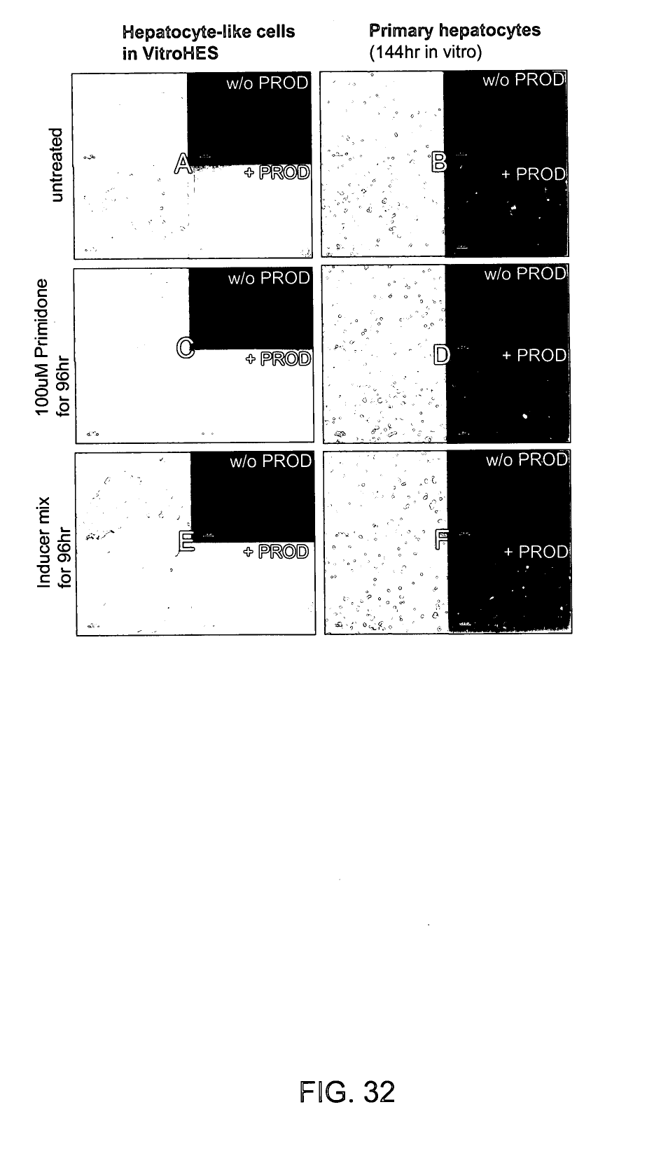

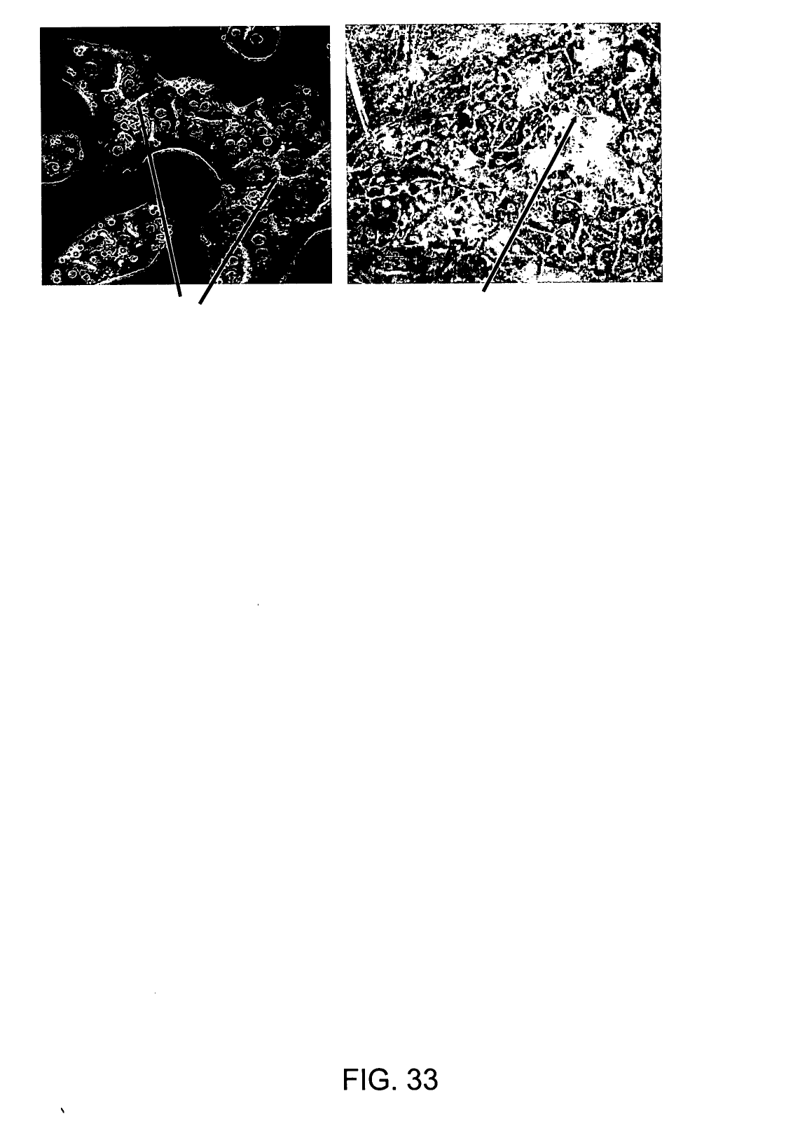

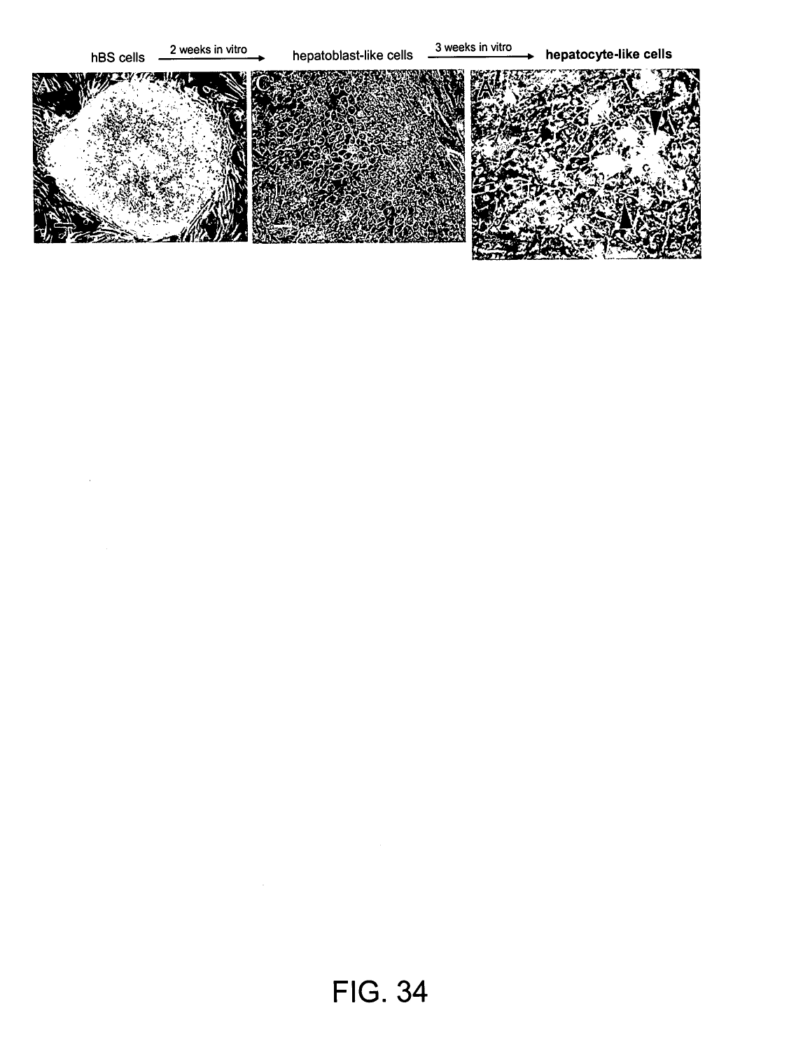

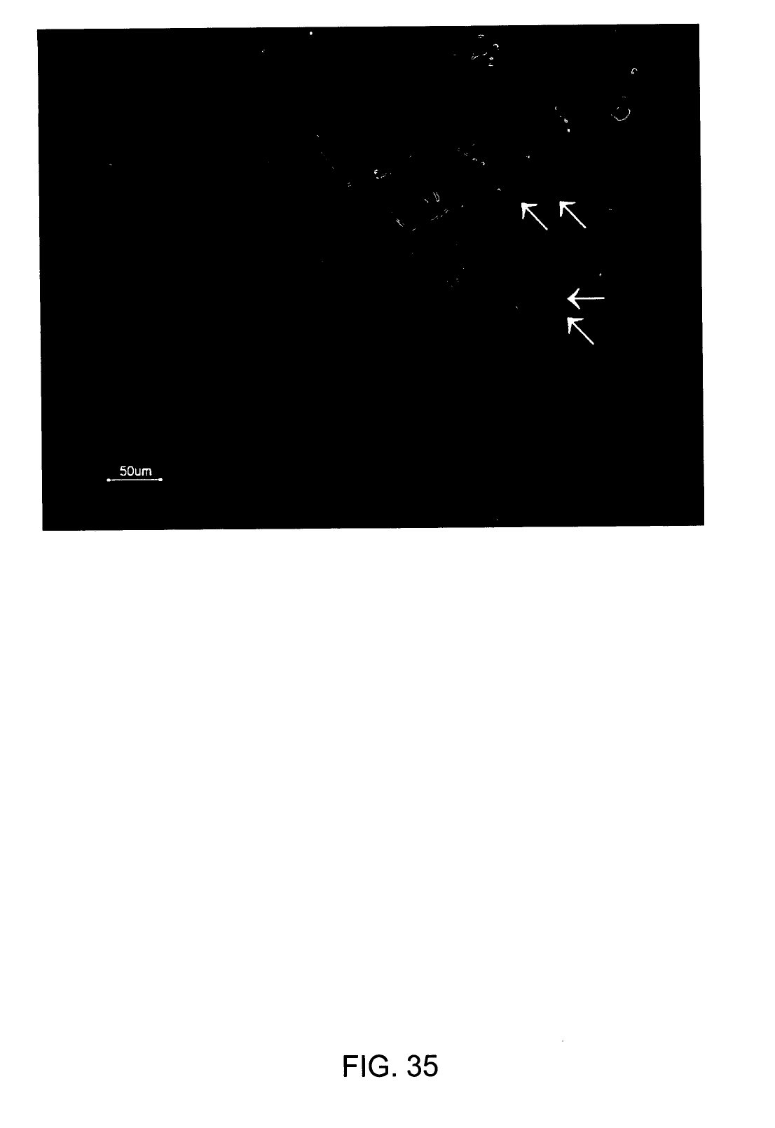

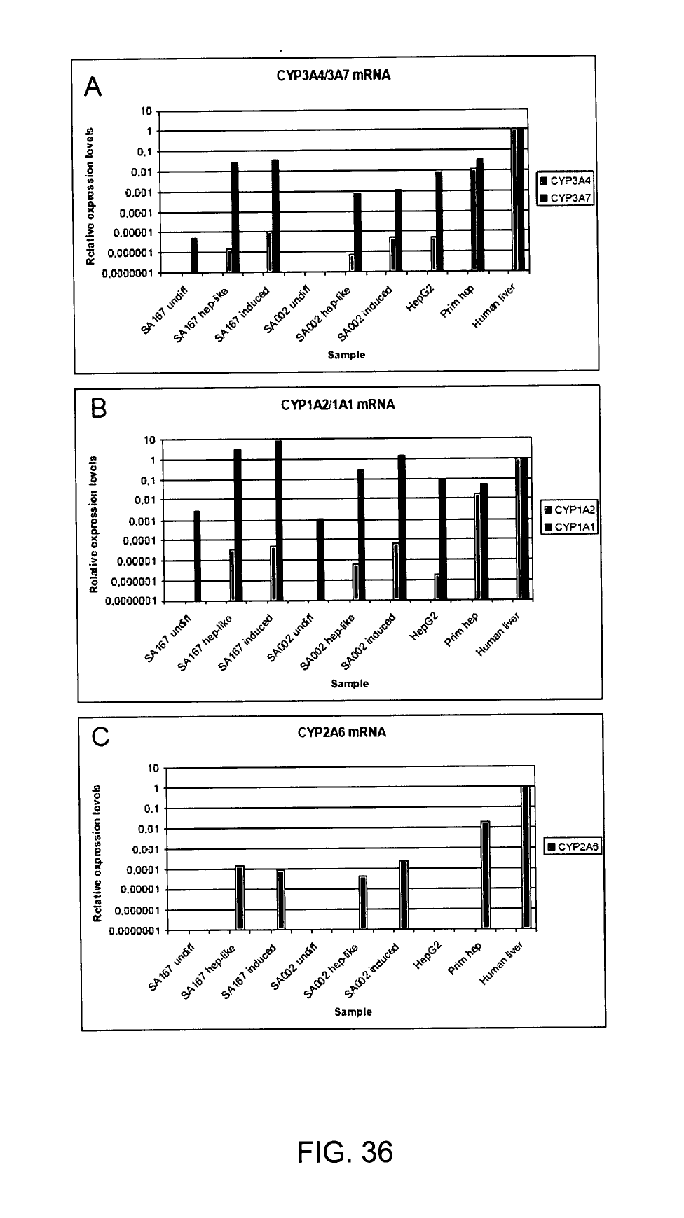

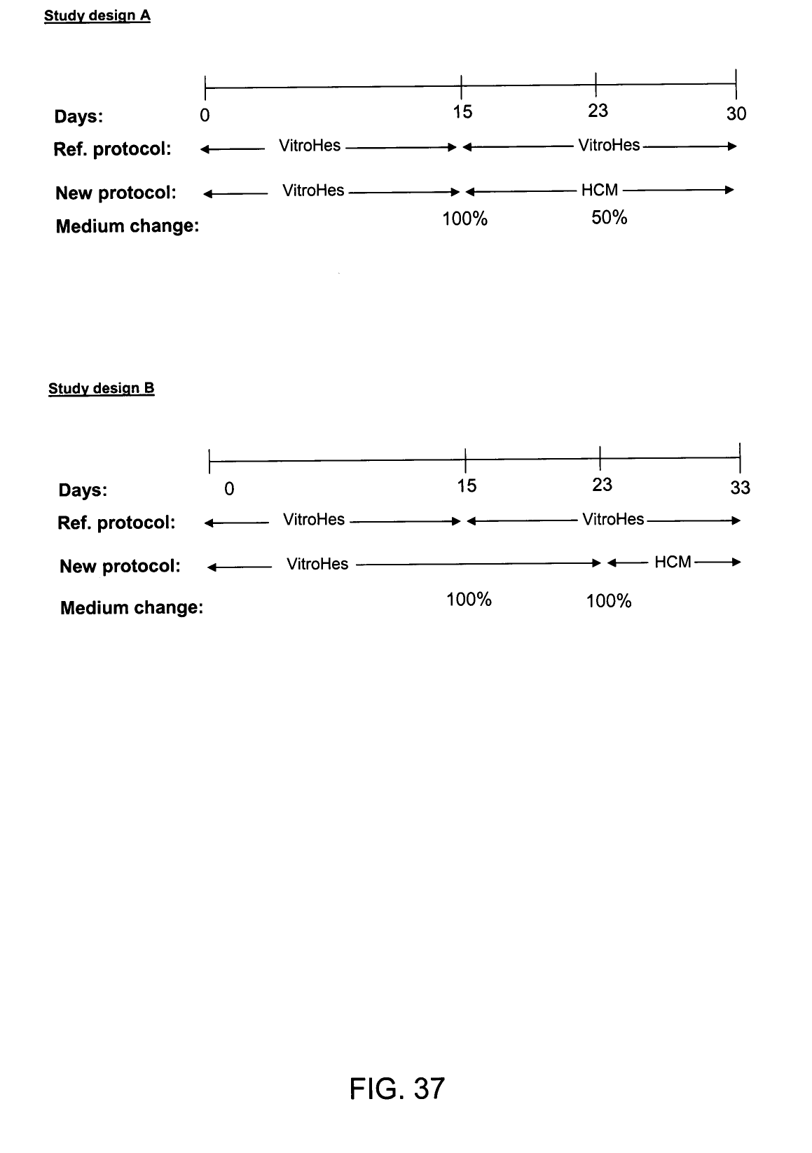

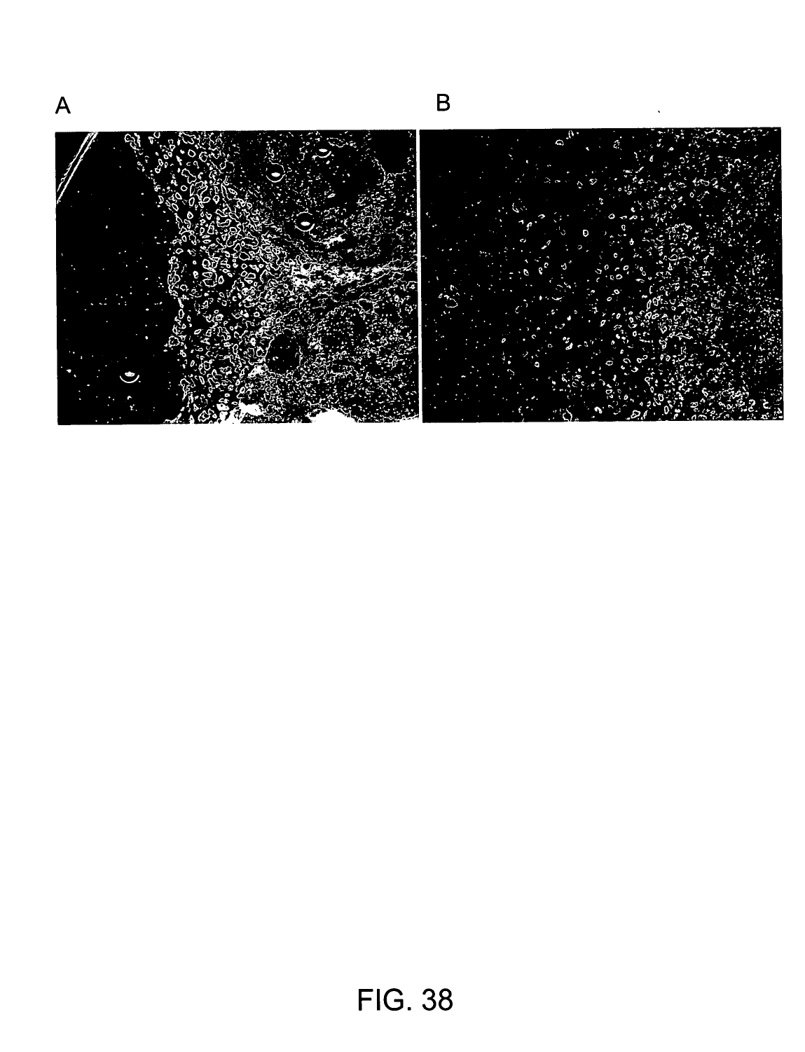

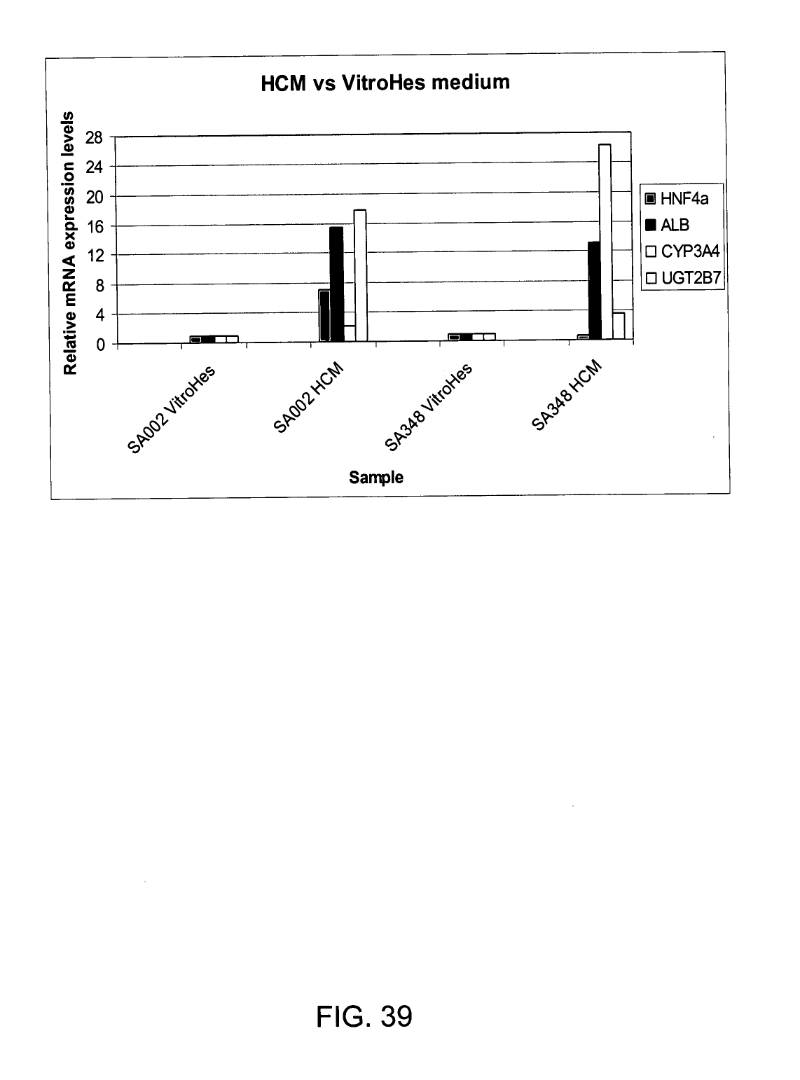

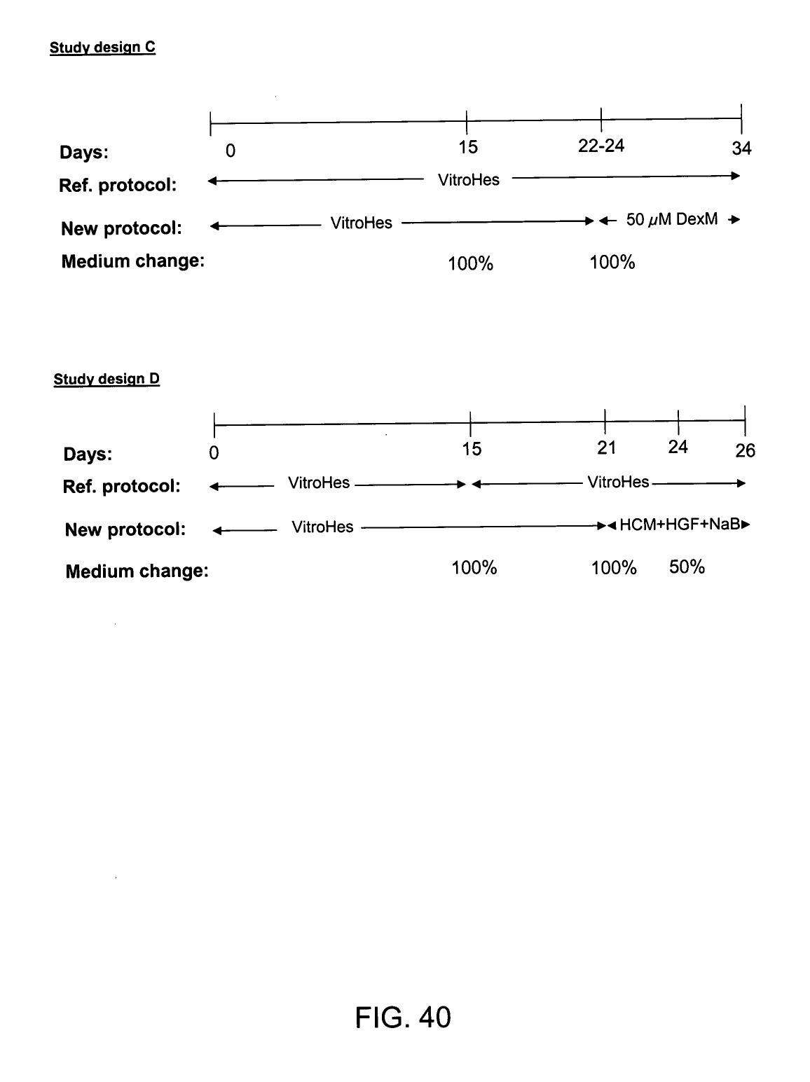

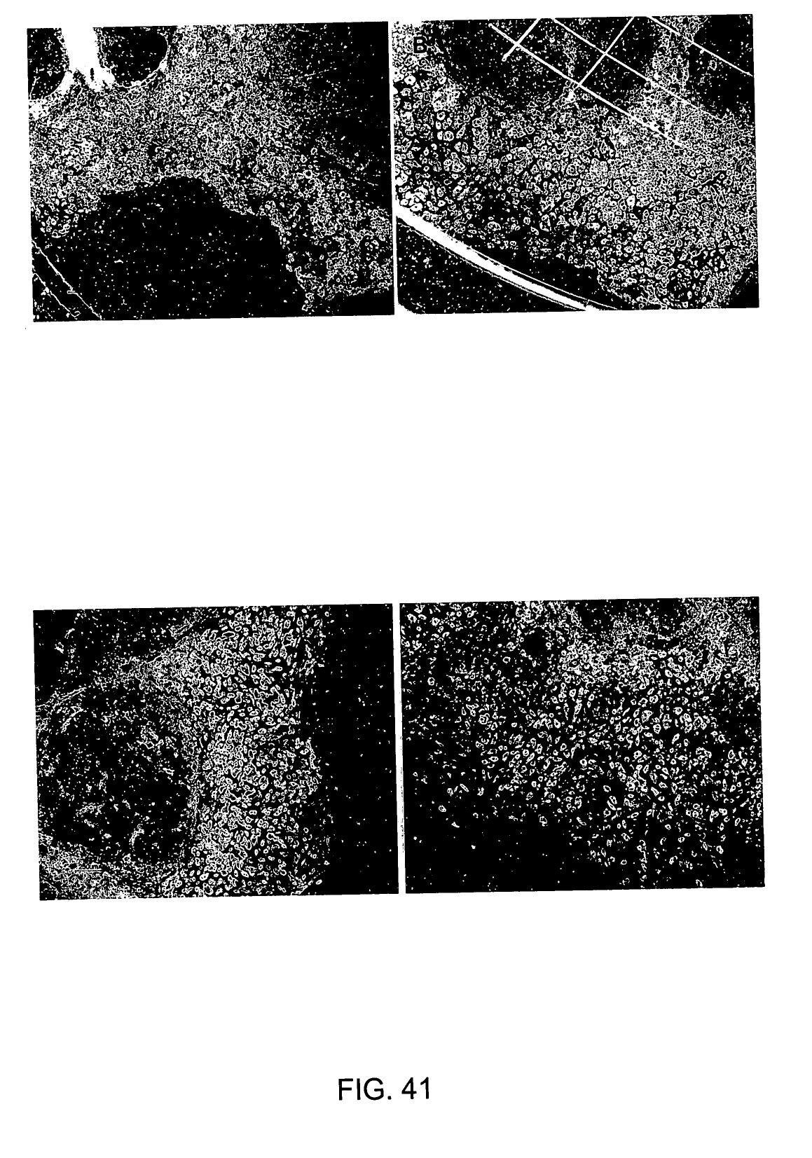

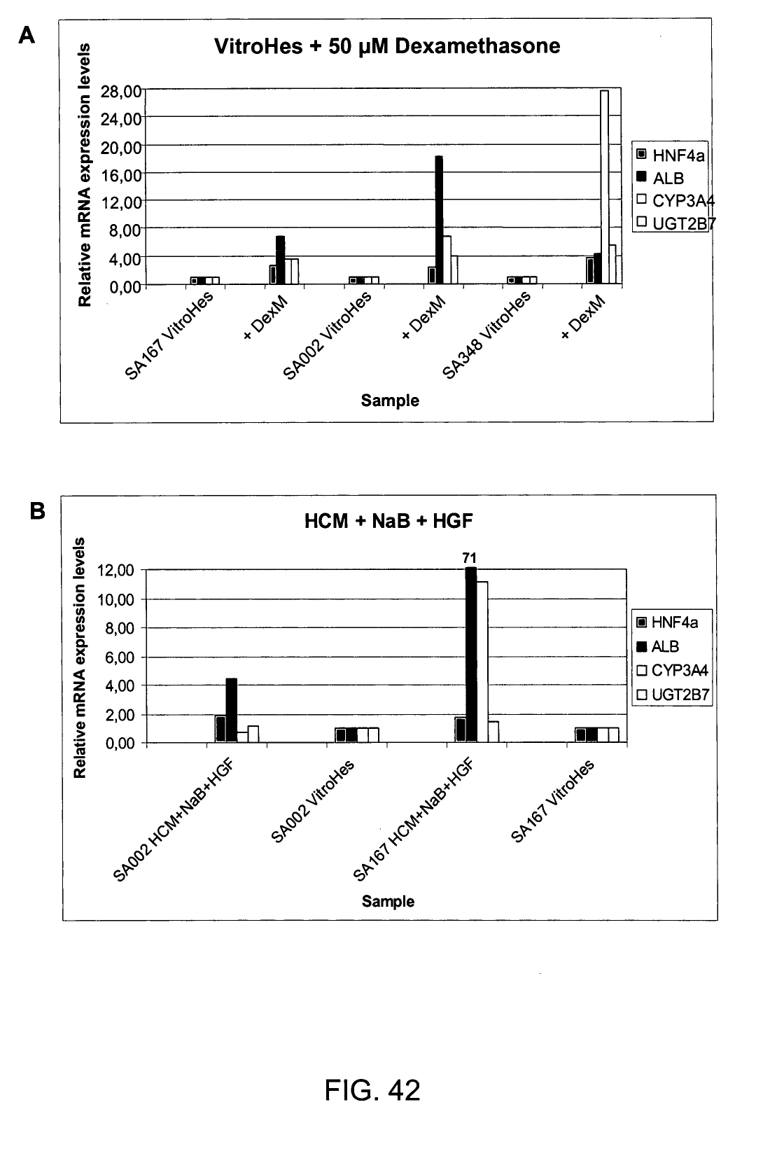

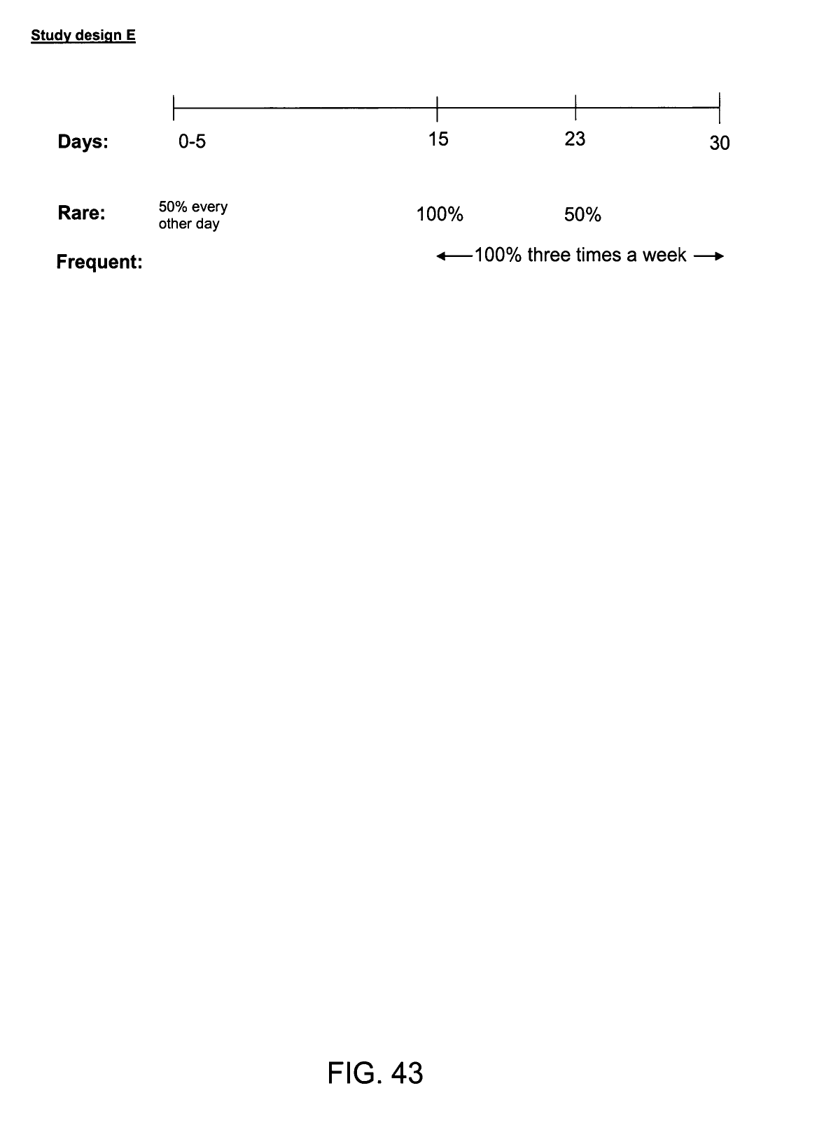

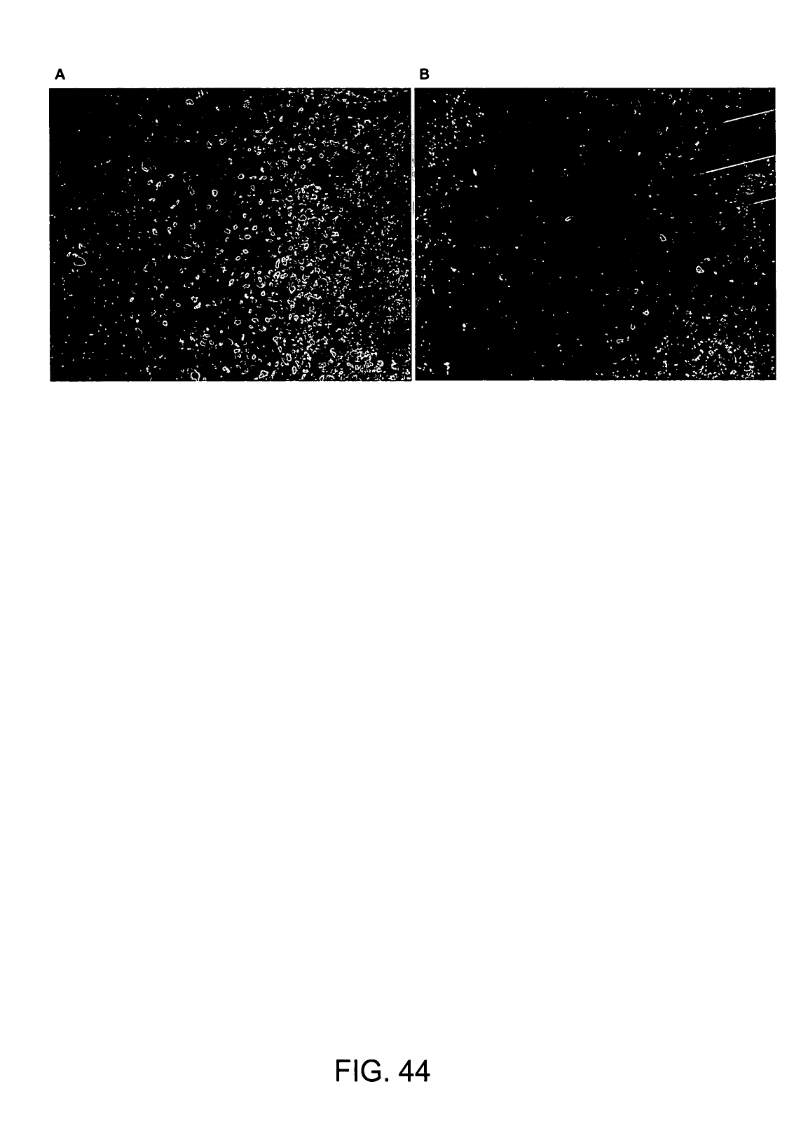

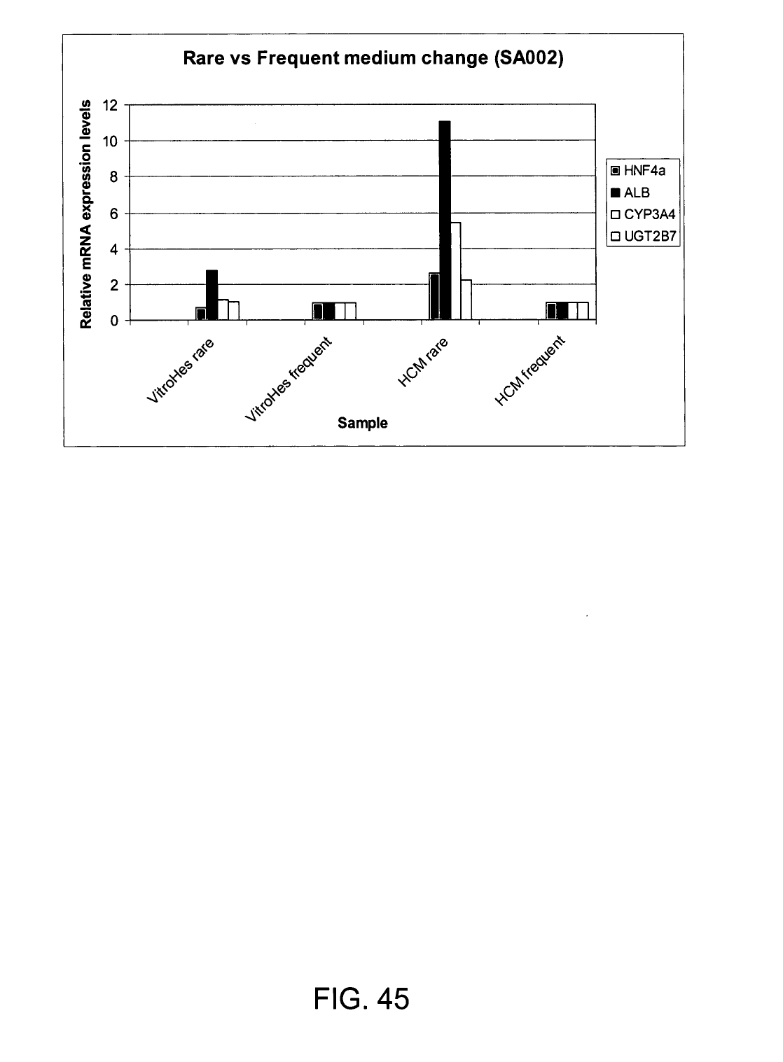

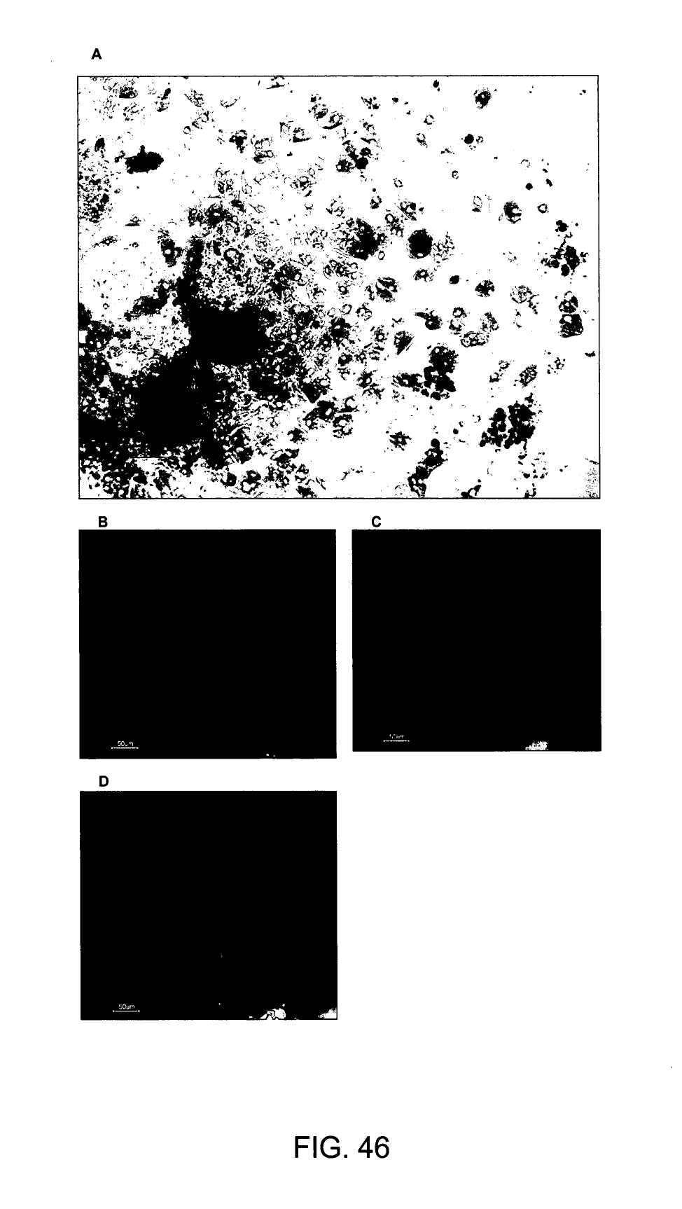

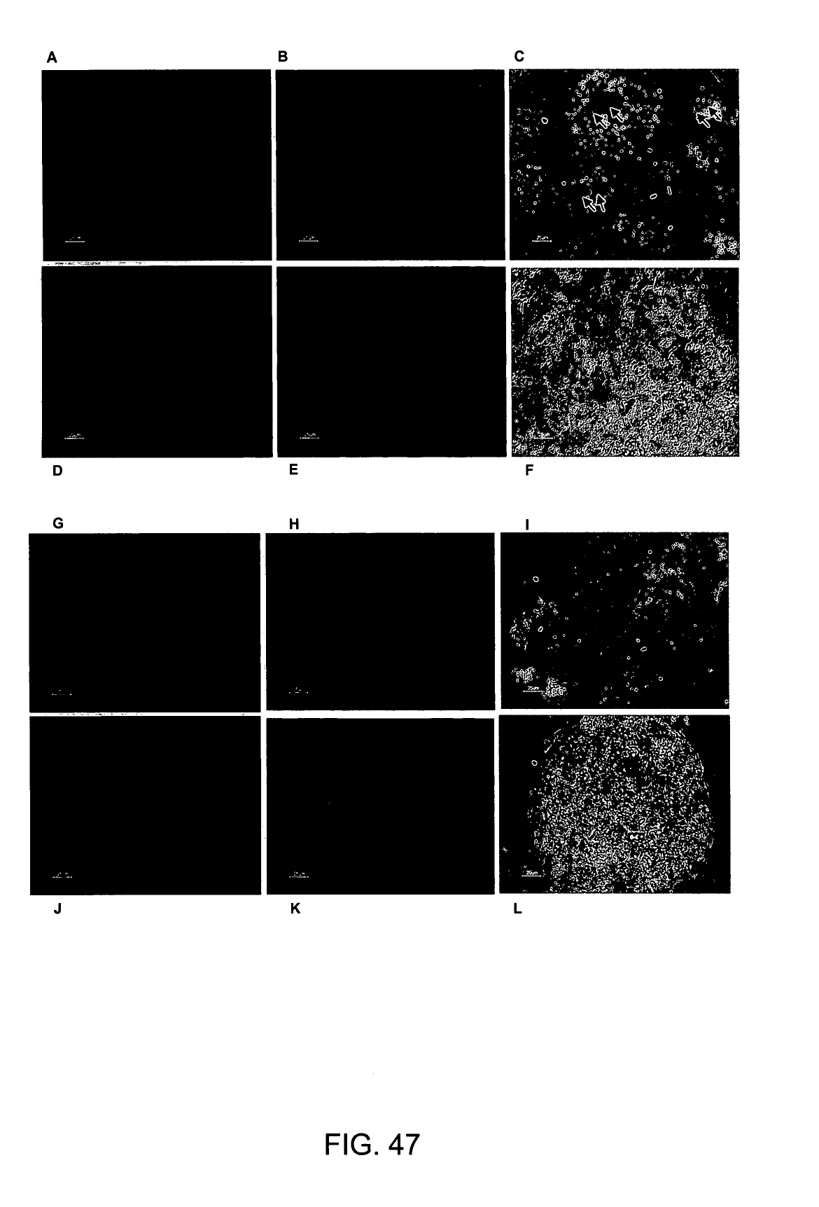

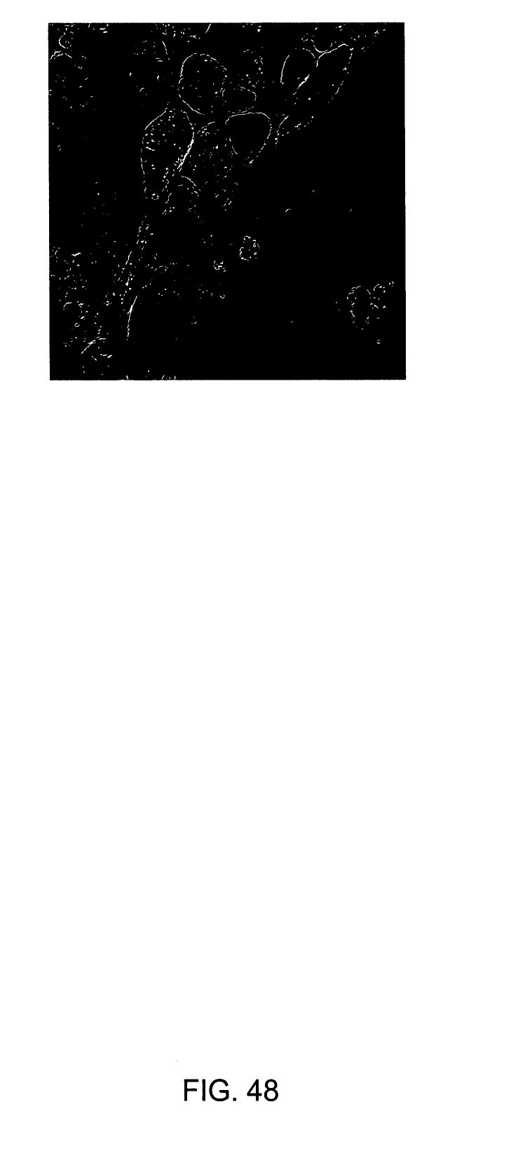

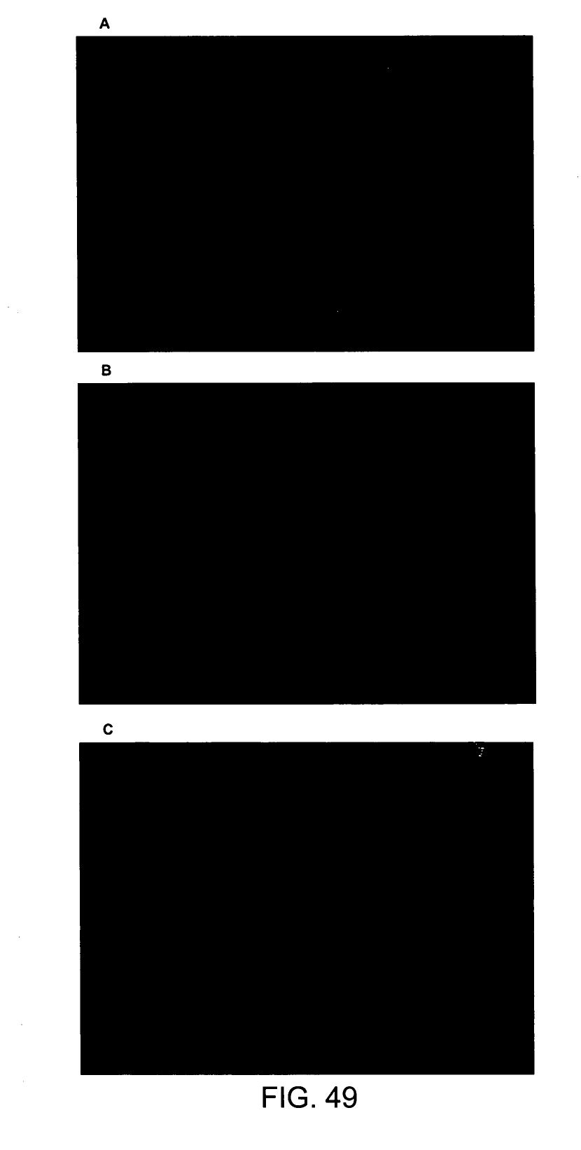

Expression of Cyp1A2 and 3A4/7 was confirmed in Western Blot (see FIG. 12). Upon treatment with Cyp inducing drugs the protein amount of 1A2 and 3A4 and or 3A7 (potential cross reaction) is clearly increased visualising the inducibility of the hepatocyte-like cells compared to protein bands from untreated and induced cells. Proteins were extracted from cells using the M-PER protein extraction reagent (Pierce), supplemented with 1:100 protease inhibitor cocktail (Sigma-Aldrich). Proteins were separated on a 12% SDS polyacrylamide gel by electrophoresis and then transferred to nitrocellulose membranes (Biorad, Hercules, Calif.). For immunoblotting, primary antibodies against (Cyp1A2 (rabbit) and Cyp3A4/7 (rabbit), both from Biomol) were diluted in 1% BSA blocking buffer. Secondary antibodies were the corresponding HRP-conjugated antibody (goat anti-rabbit, goat anti-mouse and rabbit anti-goat respectively (1:2000; DAKO, Glostrup, Denmark)). Enhanced chemiluminescence (ECL) was used according to the manufacturer's instructions (Amersham, Piscataway, N.J.). HepG2 cells were used as negative control. Primary human hepatocytes (In Vitro Technologies, Leipzig, Germany) were used as positive control.

In additional runs a Cyp1A2 specific antibody from Biomol (rabbit pAb, CR130) was used. The result from such a run on SA002, LOT AL002 and SA167, LOT AL002, induced and untreated, respectively, indicate on a specific Cyp1A2 reaction (although weak) in both hBS cell derived hepatocyte-like cell populations. (See FIG. 27).

Cyp Activity Assay (see FIGS. 13, 30, and 32):

The PROD assay showed a constitutive PROD activity in untreated hepatocyte-like cells and a strong induction of PROD activity upon treatment of the cells with Cyp inducing cocktail for 96 hours. Also EROD assay was performed indicating specific constitutive and inducible Cyp 1A2 activity (see FIG. 30). Both assays showed an activity as inducible as in primary hepatocytes and, moreover, there was in fact a basal expression, although weak, before any induction in the hBS cell-derived hepatocyte-like cells.

The cocktail contained 25 μM Rifampicin, 20 μM Desoxyphenobarbital (Primidone), 100 μM Dexamethasone, 88 mM Ethanol, 25 μM Omeprazole, and 100 μM Isoniazid. PROD (Pentoxyresorufin) is a general Cyp activity assay and EROD (Ethoxyresorufin) is specific for Cyp1 A2. PROD or EROD stock solutions (both products from Sigma Aldrich, Cat. No 77105 and 46121, respectively) are prepared in DMSO at a concentration of 2 mM. Cells were induced for 96 hr with various inducers or left untreated as controls. Before performing the assay, cells were washed carefully twice with PBS and incubated in fresh PBS for 15-30 min to wash out residual amounts of inducers. Cells were incubated with 25 μM PROD in PBS or 50 μM EROD in PBS for 60 min in the dark at 37° C. Then cells were washed again twice with PBS and analysed under the microscope.

The relative gene expression levels of Cyp3A4, 3A7, 1A1, 1A2 and Cyp2A6 were measured and compared by real-time PCR techniques in induced and non-induced cultures of hepatocyte-like cells, HepG2 and human liver extracts. Measurement of the human liver extract was set to 1 and all other samples were related to the human liver reference for each cytochrome p450. The expression for all genes is normalised against either GAPDH (CYP1A1/1A2, CYP2A6) or TBP (CYP3A4/3A7), see FIG. 36.

### Example 5

### Characterisation

Phase II Metabolic Enzymes:

Hepatocyte-like cells display a strong immunoreactivity for GST A1-1 and a weaker immunoreactivity for GST M1-1. No or very low immunoreactivity was shown for the fetal GST P1-1 (see FIG. 14 for hepatocyte-like cells differentiated on mEF.) Furthermore, hepatocyte-like cells show immunoreactivity for UGT 1A1 and UGT 1A6 (see FIG. 17). Upon treatment with inducing drugs, a slight induction is observed for GST A1-1, but no clear induction for GST M1-1 or GST P1-1. (For GST A1-1 expression in hepatocyte-like cells grown on Matrigel™, see FIG. 22.)

Used Primary Antibodies:

GSTA1-1 (rabbit) 1:500, Oxford Biomedical Research, GS62

GST M1-1 (rabbit) 1:500, Oxford Biomedical Research, GS67

GST P1-1 (rabbit) 1:500, Oxford Biomedical Research, GS72

UGT 1A1 (rabbit) 1:500, BD Biosciences, 458-411

UGT1A6 (rabbit) 1:500, BD Biosciences, 458-416

Used Secondary Antibodies:

donkey anti-rabbit-Alexa488, 1:1000, Molecular Probes, #A-21206

donkey anti-rabbit-Alexa594, 1:1000, Molecular Probes, #A-21207

Immunostaining Protocol:

15 min fixation in 4% PFA, 2×PBS wash, 30 min 5% FBS in 0.1% PBT, primary antibodies incubated in 1% FBS in 0.1% PBT overnight at 4° C., secondary antibodies in 1% FBS in 0.1% PBT for 3 hr at RT, all washes in 0.1% PBT, DAPI at 0.05 mg/ml for 5 min at RT, mounted in DAKOCytomation mounting medium.

Western Blot:

Western blot analysis confirms expression of GST A1-1 (25 kDa) in hepatocyte-like cells from hBS cell lines SA002.5 (LOT BE002.5), SA167 (LOT CE167) (see FIG. 15) and SA002 (LOT AL002) (data no shown). For the more fetal GST subtype GST P1-1 (25 kDa) no expression was detected by Western blot. Cell lysate from V79 cells over-expressing the human GSTs were used as positive controls. B-actin (42 kDa) was used as an internal loading control. Expression of GST A1-1, or GST P1-1 could not be detected in undifferentiated hBS cells from lines SA002 or SA002.5.

Proteins were extracted from cells using the M-PER protein extraction reagent (Pierce), supplemented with 1:100 protease inhibitor cocktail (Sigma-Aldrich). Proteins were separated on a 12% SDS polyacrylamide gel by electrophoresis and then transferred to nitrocellulose membranes (Biorad, Hercules, Calif.). For immunoblotting, primary antibodies against GST A1-1 and GST P1-1 (1:1000) were diluted in 1% BSA blocking buffer. Secondary antibodies were the corresponding HRP-conjugated antibody (goat anti-rabbit, goat anti-mouse and rabbit anti-goat, respectively 1:2000, (DAKO, Glostrup, Denmark)). Enhanced chemiluminescence (ECL) was used according to the manufacturer's instructions (Amersham, Piscataway, N.J.). Primary human hepatocytes (In Vitro Technologies, Leipzig, Germany) and GST protein preparations were used as positive controls.

GST Activity Assay (See FIG. 16):

GST catalytic activities toward 1-chloro-2,4-dinitrobenzene (CDNB) were determined using the spectrophotometric assay of Habig et al. 1974. CDNB (1-chloro-2,4-dinitrobenzene) is a general GST reference substrate (Mannervik 1988). The reaction mixture consisted of 85 μl of 0.1 M potassium phosphate (pH 6.5), 2.5 μl of 0.2 M GSH, 2.5 μl of 20 mM CDNB and 10 μl protein lysate in M-PER lysis buffer. A complete assay mixture with 10 μl of M-PER lysis buffer, without protein, was used as a control. Absorbance at 340 nm and 30° C. was monitored for 1 min using Philips PU8720 UV/VIS scanning spectrophotometer. The GST catalytic activity toward CDNB was assayed in hepatocyte-like cells derived from hBS cell lines SA002 (LOT AL002), SA002.5 (LOT BE002.5) and SA167 (LOT CE167) and human primary hepatocyte cultures as well as HepG2 cultures. Hepatocyte-like cells derived from hBS showed similar GST-activity as human primary hepatocytes and significant higher GST activity levels than HepG2 cultures. See FIG. 16.

### Example 6

### Characterisation

Drug Transporters

Immunohistochemistry:

Hepatocyte-like cells show immunoreactivity for the transporters MRP2, BSEP, and OATP-2 and/or OATP-8 (potential antibody cross reaction) (see FIG. 18, 19, 20). OATP-2/8 and MRP2 seem to be expressed in the majority of hepatocyte-like cells while BSEP is expressed only in a smaller population of hepatoblast- and hepatocyte-like cells.

Used Primary Antibodies:

MRP2 (rabbit) 1:50, Santa Cruz, sc-20766

BSEP (goat) 1:50, Santa Cruz, sc-17292

OATP-2 (mouse) prediluted, Progen Biotechnik GmbH, clone mMDQ

Used Secondary Antibodies:

donkey anti-goat-Alexa 488, 1:500, Molecular Probes, # A-11055

donkey anti mouse-Cy3, 1:1000, Jackson Immuno Research, #715-165-151

donkey anti-mouse-Cy2, 1:100, Jackson Immuno Research, #715-225-151

donkey anti-rabbit-Alexa488, 1:1000, Molecular Probes, #A-21206

donkey anti-rabbit-Alexa594, 1:1000, Molecular Probes, #A-21207

donkey anti-rat-Cy3, 1:500, Jackson Immuno Research, #712-165-153

donkey anti-rat-Cy2, 1:100, Jackson Immuno Research, #712-225-153

donkey anti-sheep-Alexa488, 1:1000, #A-11015

donkey anti-sheep-Alexa594, 1:1000, #A-11016

Immunostaining Protocol:

MRP2, OATP-2:

15 min fixation in 4% PFA, 2×PBS wash, 30 min 5% FBS in 0.1% PBT, primary antibodies incubated in 1% FBS in 0.1% PBT overnight at 4° C., secondary antibodies in 1% FBS in 0.1% PBT for 3 hr at RT, all washes in 0.1% PBT, DAPI at 0.05 mg/ml for 5 min at RT, mounted in DAKOCytomation mounting medium.

BSEP:

15 min fixation in 4% PFA, 2×PBS wash, 30 min 5% FBS in PBS, primary antibodies incubated in 1% FBS in PBS overnight at 4° C., secondary antibodies in 1% FBS in PBS for 3 hr at RT, all washes in PBS, DAPI at 0.05 mg/ml for 5 min at RT, mounted in DAKOCytomation mounting medium.

Analysis of MRP2 Gene Expression (See FIG. 23)

MRP2 expression in hepatocyte-like cells was between 11 and 32 times higher respectively, compared to in undifferentiated cells.

Total RNA was extracted from the hepatocyte-like cells (SA002.5, LOT BE002.5 passage 2, after 21 days of differentiation on mEF) and undifferentiated hBS cells (SA002.5, LOT BE002.5 passage 24, day 5 (control sample 1) and SA002, LOT BE002, passage 62, day 4 (control sample 2)) using the Trizol Reagent from Gibco. 5 μg of total RNA was reverse transcribed into complementary DNA (cDNA) after treatment with deoxyribonuclease I (Invitrogen Ltd, Paisley, UK) using SupercriptII kit (Invitrogen Ltd). Gene-specific primers and probes for the transporters were designed using the Primer3 software program and Netprimer. 200 ng of cDNA were amplified by polymerase chain reaction (PCR) using primers for the relevant gene with SensiMix Sybr Green Mix (1.5 mM final MgCl₂); (Celtic Diagnostics), 0.7 units AmpliTaq Gold DNA polymerase), 300 nM of gene-specific forward and reverse primers. Primer sequences used are Forward: tgcagcctccataaccatga and Reverse: ggacttcagatgcctgcca. The PCR was performed on a Corbett Rotorgene with initial steps of 2 minutes at 50° C., 10 minutes at 95° C. and then 40 cycles of 15 seconds at 95° C. and 60 seconds at 62° C. PCR amplification of each sample was performed in duplicate to minimise pipetting error. A no-template control was included for each run as a negative control. The Ct value for each sample was recorded and the relative gene expression for each sample was calculated using 2^{Δ(40-Ct)}, using the standard ABI protocol. Relative expression under assumption of 90% efficiency was estimated by setting the lowest sample to zero.

### Example 7

### Glycogen Storage Detection in Cells by PAS-Staining (Periodic Acid-Schiff Staining System, SIGMA-ALDRICH, Cat-no. 395-B)

Hepatocyte-like cells store glycogen detected by the PAS-staining method (see FIG. 21). As technical negative control saliva-treated cultures demonstrated a decrease in glycogen detection in 95-99% of the cultures. Glycogen synthesis and storage is a common function of many cell types of the human body when ever there is a glucose surplus. However, hepatocytes and skeletal muscles are in particular specialised in storing glycogen. In the differentiated hBS cell cultures before selecting or cutting out the hepatocyte-like cells other cell populations are observed that can store glycogen as well as several populations that can not store glycogen are observed. Importantly, there are non-hepatocyte-like cells in the cultures that are not storing glycogen. Cells were fixed in 4% paraformaldehyde diluted in methanol for 15 minutes at room temperature and subsequently washed three times in PBS. As technical negative control, a culture was treated with human saliva for 20 minutes at room temperature and subsequently washed in PBS. The human saliva contains α-amylase which digests glycogen. Periodic acid, which oxidizes glycols to aldehydes, was added to the treated and untreated cultures for 5 minutes at room temperature followed by repeatedly washing in PBS. Subsequently, cultures were incubated in Schiff's reagent for 15 minutes at room temperature allowing a reaction between pararosaniline and sodium metabisulfite which results in a pararosaniline product that stains the glycol-containing cellular compartments bright pink. After washing in PBS cells were counter-stained in heamatoxylin for 90 seconds at room temperature and rinsed in H₂O prior to mounting in mounting media. Heamatoxylin stains the nuclei of a cell (bluish).

### Example 8

### Genetic Analysis

Still another method to analyse specific genes of interest, e.g. those listed below is by using quantitative PCR (QPCR). One such procedure of doing that could look like the following: first suitable genes are selected, matching primers complementary to those genes are designed, quantification is done by PCR analysis and the expression levels of the genes of interest are compared to either house-keeping genes (if known to be stably expressed during differentiation) or to genes down-regulated during differentiation. Examples of suitable genes being down-regulated during differentiation in several hBS cell lines are Cripto, Nanog, and Oct-4.

The hBS cell derived hepatocyte-like cells may be further characterised at the genetic level (mRNA expression level) by conventional techniques, such as microarrays, microfluidity cards or gene chips with suitable selection of genes, such as the one listed in the table below (table 1). cDNA derived from total RNA of the samples can be hybridised with e.g. a microfluidity cards and the experiment ran in a PCR setup and further analysed using a suitable software.

TABLE 2

| | | | Transcription | |
|---|---|---|---|---|
| CYPs | UGTs | Transporters | factors | Others |
| 1A1 | 1A1 | OATP-A | Ah | Albumin |
| 1A2 | 1A3 | OATP-B | PXR | Glucose-6- |
| 1B1 | 1A4 | OATP-C | CAR | phosphatase |
| 2A6 | 1A6 | NTCP | RXR | |
| 2B6 | 1A7 | OCT1 | HNF-1alfa | |
| 2C8 | 1A8 | HPGT | HNF-4alfa | |
| 2C9 | 1A9 | MDR3 | | |
| 2C19 | 1A10 | MDR1 | | |
| 2D6 | 2B7 | BSEP | | |
| 2E1 | | MRP2 | | |
| 3A4 | | | | |
| 3A5 | | | | |
| 3A7 | | | | |
| 4A11 | | | | |
| 4B1 | | | | |

Undifferentiated hBS cells of cell line SA167, LOT CA167 and SA002, LOT AL002 as well as heptocyte-like cells derived from the two cell lines, both treated with an inducer mix and untreated were run in parallel in repeated experiments on the LDA chip following the instructor's manual (Applied Biosystems 7900HT Micro Fluidic Card Getting Started Guide) and the following shortened protocol:

cDNA was prepared from total RNA and diluted it in RNase/DNase-free water to receive a suitable concentration (see below). The following components were mixed:

cDNA (1-100 ng), 5 μl

RNase/DNase-free water

TaqMan Universal PCR, 45 μl

Master mix (2×), 50 μl

Total: 100 μl

The samples were thereafter loaded onto the LDA card (each sample mix is 100 ul and 170 ng cDNA per sample) and centrifuged, whereafter the LDA card was sealed. Finally, the card was run on ABI 7900HT real-time PCR system according to the instructions in the manual and the results analyzed by using SDS 2.2.1 software and the relative quantification method.

A summary of so far detected (by either LDA analysis or QPCR) hepatic proteins in hBS cell-derived hepatocyte-like cells are presented in table 3 and 4 below.

TABLE 3

| CYPs | UGTs | Transporters |
|---|---|---|
| 1^{a}1 | 1A1, 3, 4, 5, 6, 7, 8, 9, 10 | OATP-A |
| 1^{a}2 | 1A3 | OATP-C |
| 1B1 | 1A6 | NTCP |
| 2^{a}6 | 1A8 | OCT1 |
| 2B6 | 2B7 | MDR3 |
| 2C8 | | MDR1 |
| 2C9 | | BSEP |
| 2C19 | | MRP2 |
| 2D6 | | |
| 2E1 | | |
| 3A4 | | |
| 3A5 | | |
| 3A7 | | |

TABLE 4

| Transcription factors | Others |
|---|---|
| PXR | Albumin |
| CAR | Glucose-6- |
| FXR | phosphatase |
| RXRα | Apolipoprotein E |
| RXRβ | Alcoholdehydrogenase |
| RXRγ | 1A |
| HNF-1α | |
| HNF-3α | |
| HNF-3β | |
| HNF-4α | |
| HNF6 | |
| DBP | |
| C/EBP A | |
| C/EBP B | |

### Example 9

### Bioreactor Culture of Hepatocyte-Like Cells

The hBS cells and derivative cell types thereof, such as hepatoblast-like cells or hepatocyte-like cells may be grown in a 3-D space which may be compartmentalized by e.g. artificial hollow fiber capillary membranes. This system would enable growth of the cells into larger cell masses between the capillaries, and provide an optimized, natural environment by the perfusion of culture medium and gases like oxygen. The closed bioreactor systems may be developed to produce larger amount of cells (up to 10ˆ11 cells), and to support the maintenance of functional properties of the cells. Cell isolation via enzyme perfusion would then allow scale-up in closed GMP systems. Cell purification could then be performed using e.g. FACsorting based on e.g. membrane antigen expression or using density gradient media and centrifugation.

### Example 10

### Urea Synthesis

Urea synthesis is a liver specific characteristic therefore the urea levels of the medium from different cell-lines differentiating into hepatocyte-like cells were measured at different time points after 24 h incubation. Medium samples were collected and sent for analysis. Urea secretion was analyzed using a kit for kinetic UV assay for urea/urea nitrogen (Roche/Hitachi) at Klinisk Kemi, C-lab, Sahlgrenska University Hospital, Gothenburg.

Expected values in serum/plasma are 10-50 mg/dL (1.7-8.3 mmol/L). The lower level for urea detection according to the method was 0.8 mmol/L

| Cell line and age | |
|---|---|
| of culture | urea mmol/L |
| SA002 day 5 | <0.8 |
| SA002 day 10 | <0.8 |
| SA002 day 20 | 1.1 |
| SA002 day 28 | 1 |
| SA002.5 day 12 | <0.8 |
| SA002.5 day 26 | 1.3 |
| SA167 day 5 | <0.8 |
| SA 167 day 10 | 0.8 |
| SA167 day 20 | 0.8 |
| SA167 day 28 | 1 |
| Human primary | 1.15 |
| hepatocytes | |

hBSC-derived hepatocyte-like cells produce and secretes urea into the medium at levels similar to primary hepatocytes. Interestingly enough, significant urea levels can only be measured after hepatocyte-like cells are appearing within the cultures, around day 20 and later.

Further functional testing regarding e.g. albumin secretion and can be performed as described in Schwarz et al., 2002 and 2005. Still further functional characterisation of the hepatocyte-like cells and hepatoblast-like cells could be analysed for their ability to perform LDL uptake and glyconeogenesis.

### Example 11

### Cell Polarity and Functionality

Sandwich culture systems can further improve cell polarity and functionality over time of hepatocyte-like cells. In such culture system, embedding of the hepatocyte-like cells provides, other than lower levels of oxidative stress, a 3D environment mimicking the liver in vivo, whereby the hepatocyte-like cells may show polarity, i.e. form an epithelium-like structure with an apical side (hydrophobic, towards the bile side in vivo) and a basolateral side (hydrophilic, towards the blood side in vivo). Another potential improvement with hepatocyte-like cells in sandwich cultures is even stronger inducibility of Cyp expression.

### Example 12

### Notch Characterization with Immunohistochemistry

Cultured hBS cells grown in IVF dishes were fixed with 4% paraformaldehyde for 15 min at room temperature. The cells were then incubated with primary antibody (Notch2, 1:200, Santacruz, sc5545, USA) overnight at 4° C. The next day the cells were washed with 1×PBS twice for 10 min and subsequently incubated with secondary antibody (anti-rabbit-FITC, 1:500) in 1×PBS at room temperature for 1 h. washed twice for 5 min each and exposed to 1 μg/ml DAPI solution for 5 min. After washing the cells twice for 5 min each with water they were mounted in Aquamount and analyzed by fluorescence microscopy. Recently it has been shown that undifferentiated hBS cells express Notch2 (Noggle et al., 2006). In Cellartis hBS cell line SA002 the vast majority of cells are in the first days of differentiation weakly Notch2 positive. After two weeks Notch2 is found in only a few subsets of hepatocyte-like and/or hepatoblast-like cells, i.e. binucleated cells that resemble hepatic morphology (see FIG. 35).

### Example 13

The proliferative status of hepatoblast-like and hepatocyte-like cells was tested by culturing hBS cells for 14 and 21 days, respectively, according to the intrinsic factor protocol in Example 2. The day of analysis the cultures were fixed in 4% PFA for 15-20 minutes in room temperature, washed several times in PBS and permeabilized in 0.1-0.5% TritonX100 diluted in PBS. For hepatoblast-like cells, the endodermal/early liver marker HNF4α (rabbit pAb, Santa Cruz, SC-8987, 1:400 dilution) was used and for hepatocyte-like cells, the endoermal/liver marker HNF3beta (Foxa-2) (IgG, Santa Cruz, SC-6554, 1.200 dilution) was used. For studying proliferation of the two hepatic-like cell populations the antibody, Ki67 (BD Pharmingen, #556003, 1:500 dilution) was added for over-night incubation in the fridge. After several washes secondary antibodies were applied and incubated for 2 h in room temperature. Cultures were washed and DAPI was included in the last wash in order to detect nuclei. Hepatoblast-like cells positive for HNF4α are proliferating indicated by co-localization with Ki67, (see FIG. 28). Hepatocyte-like cells were not positive for Ki67, indicating non-proliferating cells.

In addition was tested the induction of proliferation of hBS cell-derived hepatocyte-like cells to see if a proliferative capacity could be induced in the hepatocyte-like cells by changing media conditions and adding growth factors known to stimulate proliferation in primary hepatocytes. hBS cells (cell line SA002) were allowed to differentiate for 17 days into hepatoblast- and hepatocyte-like cells under previously describe conditions. Media was changed into Williams medium E (Sigma, W-4128) supplemented with 10% FBS (or Serum Replacement), 1% PEST, 1% Glutamax, 1×ITS, 0.25 ng/ml Dexamethasone, 2% DMSO, 20 ng/ml HGF, 10 ng/ml EGF, 10 ng/ml Oncostatin M, 10 mM Nicotinamide and 4 ng/ml bFGF. hBS cells were grown for another 5-15 days with the medium being changed approximately every 5 days. The proliferative capacity of the cells was thereafter analysed after an additional 11 days as described above. The hepatocyte-like cells derived from hBS cells proliferated when cultured in Williams E-media supplemented as described above, while no proliferation was detected in hepatocyte-like cells grown in VitroHES™ supplemented with 4 ng/ml bFGF (see FIG. 29). Accordingly, the hepatocyte-like cells can be stimulated to an increased proliferative capacity if cultured in therefore suitable media.

### Example 14

### Differentiation of hBS Cells to Hepatocyte-Like Cells in 96-Well Plates

Four to five days old hBS cells were manually dissected into small pieces and transferred to mitomycin C-treated mEF-coated 96-wells. Two to three pieces of hBS cells were added to each well. The cultures were incubated in VitroHES™ supplemented with 4 ng/ml bFGF at 37° C., 5% CO₂ and 95% humidity for 20-30 days. 50% of the medium was replaced with fresh VitroHES™ supplemented with 4 ng/ml bFGF at day 10 and 100% at day 20. The hBS cell colonies differentiated in a similar pattern as hBS cells-colonies cultured in MEF-coated IVF-dishes, thus hepatoblast-like cells, immuno-reacted positively with HNF4alpha appeared in the periphery of the colony around day 14 and hepatocyte-like cells at day 20.80% of the 96-wells were successful in differentiating hBS cells into hepatocyte-like cells at day 20 by using this protocol.

### Example 15

### Reseeding of Hepatocyte-Like Cells onto Different Surfaces

Prior to reseeding of hepatocyte-like cells, wells of a 96-well plate were pre-coated with different coatings; collagen I from rat tail (BD Biosciences, #354236), different concentrations of Matrigel (basement membrane matrix, BD Biosciences, #356237) ranging from 10% to 100%, or mitocycin C treated mEF cell layer. Coatings were performed as follows: Collagen I was added to 96-wells and incubated at 37° C., 5% CO₂, 95% humidity for 30 min-1 h. The surplus of the collagen I was discarded from each well and the wells were subsequently washed twice with PBS to get rid of any acidic acid traces. The wells were filled with 50-100 μl of pre-warmed HCM-medium (HBM™, cc-3199 supplemented with HCM™ SingleQouts of cc-4316BB, cc-4362BB, cc-4335BB, cc-4313BB, cc-4321BB, cc-4317BB, cc-4381BB from Cambrex) supplemented with 20% FCS. For matrigel-coating, frozen aliquots of matrigel were defrosted overnight in the fridge. The matrigel was diluted in HCM-medium to achieve 10% to 100% matrigel. Different concentrations of matrigel was added to 96-wells and incubated at 37° C. for 30 min. The surplus of the matrigel was discarded before adding HCM-medium supplemented with 20% FCS. Regions of hepatocyte-like cells from 25 to 38 days old cultures were micro dissected by using micro scalpels from BD and transferred with a stem cell knife to a collecting dish containing VitroHES™ medium. The micro dissected hepatocyte-like cells containing regions formed small clusters and were gathered from several plates. The clusters were either treated with calcium and magnesium free PBS for 10 min at 37° C., TrypLe Select (Gibco, #12563) on a heating plate, 42° C., for 3-5 min or Collagenase IV for 5-15 min at 37° C. before reseeding onto different coatings of a 96-well plate containing HCM-medium supplemented with 20% FCS. After one day, clusters with hepatocyte-like cells had attached to the surface and started to migrate from the cluster onto the surface. This was true for all different kinds of coatings and dissociation treatments of the hepatocyte-like cells. HCM-medium supplemented with 20% FCS was exchanged to serum-free HCM-medium. Cultures were provided with fresh HCM-medium every other day. After several days the hepatocyte-like cells had reseeded the wells. The hepatocyte-like cells were kept for 45 days in culture with retained liver typical morphology; large polygonal and multi nucleated cells (see FIG. 46) and were positive for the hepatocyte markers HNF3beta and CK18 (FIG. 46)

### Example 16

### Metabolising Hepatocyte-Like Cells

hBS cells derived hepatocyte-like cells obtained by different differentiation protocols was tested for its ability to metabolize Phenacetin (Aldrich), Diclophenac (SIGMA) and Midazolam (SIGMA) via the phase I cytochrome p450 enzymes, cyp1A2, cyp2C9 and cyp3A4 respectively in the absence of inducers. Released metabolites of respective substance into the medium were measured by LC-MS. Mixed cultures containing hepatocyte-like cells from cell-line SA002, SA002.5 and SA348 were tested at an age of 26 to 35 days. The substances were incubated as a cocktail in phenol red free medium; 26 μM Phenacetin, 9 μM Diclofenac and 3 μM Midazolam for 6 h, 12 h and 24 h at 37° C. and 5% CO₂ Samples from each culture and time point were collected and centrifuged for 20 min at high speed to get rid of any cell debris. 100 μl of the cleared medium sample was transferred to a 96-well plate and 15 μl of acetonitril was added to each well. The samples were frozen until metabolite measurements were performed and analysed by LC-MS.

The liquid chromatographic system consisted of an HP 1100 series LC pump and column oven (Agilent Technologies Deutschland, Waldbronn, Germany) combined with an HTS PAL injector (CTC Analytics, Zwingen, Switzerland). For, 4-hydroxydiclofenac and 1-hydroxymidazolam LC separations were performed on a reversed-phase HyPurity C18 (2.1×50 mm, 5 μm, ThermoQuest, Runcorn, UK) at 40° C. with a HyPurity C18 precolumn. The mobile phase consisted of (A) 0.1% (v/v) formic acid and (B) 0.1% (v/v) formic acid in acetonitrile. The organic modifier content was increased linearly from 5 to 80% B over 3 min, then back to 5% B for 0.2 min.

For paracetamol chromatography was performed on a Zorbax Eclipse XDB-C8 (4.6×150 mm, 5 μm) with a HyPurity C18 precolumn, employing the same system and mobile phase. The organic modifier content was increased linearly from 2 to 30% B over 5 min, then from 30 to 80% over 2 min, and then back to 2% B for 0.1 min. The retention times for 4-hydroxydiclofenac, 1-hydroxymidazolam, and paracetamol were 2.9, 2.4 and 6.4 min, respectively. Detection was performed with a triple quadrupole mass spectrometer, API4000, equipped with electrospray interface (Applied Biosystems/MDS Sciex, Concord, Canada). The MS operated at turbo heater temperature at 450° C. for 4-hydroxydiclofenac and at 550° C. for 1-hydroxymidazolam and paracetamol, nebuliser gas was (GS1) 50, 30, 70, and 50, respectively. Turbo gas was (GS2) 50, 60, 70, and 70, respectively, and curtain gas was 20, 20, 10, and 20, respectively. Electrospray voltage was −3 kV in negative mode for 4-hydroxydiclofenac, and 5 respective 3.5 kV in positive mode for 1-hydroxymidazolam and paracetamol. The collision energy was set at −15, 39, and 21 V, respectively, and collision-activated dissociation gas at 5, 7, and 5, respectively. The MRM transitions chosen were 309.9>265.9 for 4-hydroxydiclofenac, 342.0>202.7 for 1-hydroxymidazolam, and 152.3>110.0 for paracetamol. A dwell time of 200 ms was used. Instrument control, data acquisition and data evaluation were performed using Applied Biosystems/MDS Sciex Analyst 1.4 software.

Cyp-activity of different hBS cell derived hepatocyte-like cells from different ages and in the absence of inducers.

| Cell | | | | | |
|---|---|---|---|---|---|
| line | Age | Cyp1A2 | Cyp3A4 | Cyp2C9 | Comments |
| SA002 | 26-37 | x | x | x | |
| | days | | | | |
| SA348 | 30-37 | x | x | x | |
| | days | | | | |
| SA002.5 | 34-39 | x | x | x | Reseeded HCLC to |
| | days | | | | collagen I or |
| | | | | | matrigel showed |
| | | | | | Cyp 1A2, 3A4 and |
| | | | | | 2C9 activity. |

### Example 17

### Composition of cyp Activity

The composition of cyp-activity within a hepatocyte is essential when analysing drug metabolism in order to predict drug metabolism in vivo. To analyse how well the cyp-activity composition of the hBSC-derived hepatocyte-like cells mirrors that of human hepatocytes, a cocktail of the drugs Phenacetin (26 μM), Diclophenac (9 μM) and Midazolam (3 μM), (metabolised by cyp1A2, cyp2C9 and cyp3A4 respectively) were added to the hepatocyte-like cell derived from the cell line SA348 and human primary hepatocytes. After 12 h incubation of the drug cocktail samples were collected for LC-MS detection of the metabolites. The samples were prepared and analysed by LC-MS as previous been described in example 16. The cyp activity composition was analysed between the three enzyme systems tested.

Cyp-activity composition between Cyp1A2, Cyp3A4 and Cyp2C9

| | Cyp1A2 | Cyp3A4 | Cyp2C9 |
|---|---|---|---|
| Human primary hepatocytes | 67% | 21% | 12% |
| Hepatocyte-like cells SA348 | 70% | 21% | 8% |

The cyp activity composition of the hepatocyte-like cells was similar to the cyp activity composition in human primary hepatocyte cultures.

### Example 18

### Improvements of Hepatocyte-Like Cells

In order to improve the hBSC-derived hepatocyte-like cells a screening method based on real-time PCR techniques was used. The relative expression levels of four genes; HNF4 alpha, Cyp3a4, Albumin and UGT2B7 indicated the improvements of the hepatocyte-like cells. Cyp3a4 is an important cytochrome p450 enzyme in the adult liver. It constitutes 60% of the phase I enzymes activities in the adult liver. Albumin is highly expressed in mature and adult hepatocytes. UTG2B7 is an important phase II enzyme (glucuronosyltransferase) of the adult liver. Rising levels of Cyp3a4, Albumin and UTG2B7 indicate improved, matured and potentially more functional hepatocyte-like cells. HNF4alpha is an early marker for hepatoblast-like cells. A rising level in HNF4alpha gene expression indicates an increased number of hepatoblast-like cells. High levels of Albumin, Cyp3A4 and UTG2B7 and in addition a decreased level of HNF4alpha will indicate a mature and more functional population of HCLC.

Total RNA of cell samples from different protocols (see below for detailed description) was extracted using Invitrogens Trizol method. cDNA was then made from the total RNA samples using oligo dT. Thereafter the cDNA was amplified in a real-time PCR reaction using ready-to-use Taqman primers, standard program and apparatus from Applied Biosystems.

For analysis the numbers of cycles (CT-value) needed to detect a PCR product was normalized to the CT-value of a house-keeping gene, GAPDH, in the same sample. The normalized CT-value was thereafter compared with normalized CT-value of the control sample in each experiment by using the comparative Cₜ method for relative quantification (ΔΔCₜ method). The results were then presented as a fold change between a sample and the control sample in the same experiment.

Different parameters and culturing protocols were elaborated with in order to improve hepatocyte-like cells. Different mediums were tested, hepatocyte-like cells were kept for longer time in cultures, frequency in medium replacement was analysed, different maturation and induction factors were tested etc.

The reference protocol for differentiating hBS cells into hepatocyte-like cells (similar to what is described in example 2):

| | |
|---|---|
| Day 1 | hBS cells were manually cut into pieces and transferred |
| | to mEF-coated IVF-dishes containing VitroHES ™ -medium |
| | supplemented with 4 ng/ml bFGF. |
| Day 10 | 50% of the medium was replaced with VitroHES ™ |
| | supplemented with 4 ng/ml bFGF. |
| Day 14-15 | Hepatoblast-like cells indicated by HNF4-alpha staining |
| | is present in the periphery of the colony. |
| Day 20 | 100% of the medium was replaced with VitroHES ™ |
| | supplemented with 4 ng/ml bFGF. Hepatocyte-like cells are |
| | appearing in the periphery of the colony. |

One of the key-elements of the protocol is the very rare occurring replacement of medium. Intrinsic factors are crucial for the differentiation process.

Improved protocols

| I | Different mediums | |
|---|---|---|
| | HCM-medium from Cambrex | Exp. 1, 2 in Table 5; |
| II | Supplementation with maturation and induction factors | |
| | a. Dexamethasone | Exp. 1, 2, 3 in Table 6; |
| | b. HCM-medium, HGF, Sodium Butyrate | Exp. 1, 2 in Table 7; |
| III | Medium replacement frequency | |
| | HCM-medium and VitroHES ™ | Exp. 1 in Table 8; |
| | | FIG. 43-45 |

TABLE 5

| | | | | | | | | Medium | | | Real- |
|---|---|---|---|---|---|---|---|---|---|---|---|
| Exp | Cell line | | | | Supplements/ | | | replacement | Study | Mor- | time |
| # | & passage | Aim | Groups | Medium | conc. | Surface | Duration | frequency | design | phology | PCR |
| 1 | SA002 p55 | Test medium | A | VitroHes | 4 ng/ml bFGF | MEF | 15 d | 50% at d 23 | A, FIG. | FIG. | FIG. |
| | fr 070110 | from Cambrex, | | | | | Vitrohes + | | 37 | 38A | 39 |
| | | HCM | | | | | 15 days | | | | |
| | | | | | | | VitroHes | | | | |
| | | | B | HCM | 1% PEST | MEF | 15 d | 50% at d 23 | A, FIG. | FIG. | FIG. |
| | | | | | 1% Glutamax | | Vitrohes + | | 37 | 38B | 39 |
| | | | | | 4 ng/ml bFGF | | 15 days | | | | |
| | | | | | | | HCM | | | | |
| 2 | SA348 | Test medium | A | HCM | | MEF | 23 d | 100% d 23 | B, FIG. | FIG. | FIG. |
| | fr. 31/1-07 | from Cambrex, | | | | | Vitrohes + | 2.5 ml | 37 | 38B | 39 |
| | | HCM | | | | | 10 d | | | | |
| | | | | | | | HCM | | | | |
| | | | B | VitroHes | 4 ng/ml bFGF | MEF | 33 d | 100% d 23 | B, FIG. | FIG. | FIG. |
| | | | | | | | VitroHes | 2.5 ml | 37 | 38A | 39 |

TABLE 6

| | | | | | | | | Medium | | | Real- |
|---|---|---|---|---|---|---|---|---|---|---|---|
| Exp | Cell line | | | | Supplements/ | | | replacement | Study | Mor- | time |
| # | & passage | Aim | Groups | Medium | conc. | Surface | Duration | frequency | design | phology | PCR |
| 1 | SA167 p28 | Test VitroHes | A | VitroHes | 4 ng/ml bFGF | MEF | 32 d Vitrohes | 100% day 15 | C, FIG. | FIG. | FIG. |
| | fr 070122 | with | | | | | | and day 24 | 40 | 41A | 42A |
| | | Dexamethasone | | | | | | | | | |
| | | (DexM) | | | | | | | | | |
| | | (dissolved in | | | | | | | | | |
| | | DMSO) | | | | | | | | | |
| | | | B | VitroHes | 4 ng/ml bFGF + | MEF | 24 d Vitrohes + | 100% day 15 | C, FIG. | FIG. | FIG. |
| | | | | | 50 uM DexM | | 8 days | and day 24 | 40 | 41B | 42A |
| | | | | | | | VitroHes/DexM | | | | |
| | | | C | VitroHes | 4 ng/ml bFGF + | MEF | 24 d Vitrohes + | 100% day 15 | C, FIG. | | FIG. |
| | | | | | 0.1% DMSO | | 8 days | and day 24 | 40 | | 42A |
| | | | | | | | VitroHes/DMSO | | | | |
| 2 | SA002 p53 | Test VitroHes | A | VitroHes | 4 ng/ml bFGF | MEF | 32 d Vitrohes | 100% day 15 | C, FIG. | | FIG. |
| | fr 070122 | with | | | | | | and day 24 | 40 | | 42A |
| | | Dexamethasone | | | | | | | | | |
| | | (DexM) | | | | | | | | | |
| | | (dissolved in | | | | | | | | | |
| | | DMSO) | | | | | | | | | |
| | | | B | VitroHes | 4 ng/ml bFGF + | MEF | 24 d Vitrohes + | 100% day 15 | C, FIG. | | FIG. |
| | | | | | 50 uM DexM | | 8 days | and day 24 | 40 | | 42A |
| | | | | | | | VitroHes/DexM | | | | |
| | | | C | VitroHes | 4 ng/ml bFGF + | MEF | 24 d Vitrohes + | 100% day 15 | C, FIG. | | FIG. |
| | | | | | 0.1% DMSO | | 8 days | and day 24 | 40 | | 42A |
| | | | | | | | VitroHes/DMSO | | | | |
| 3 | SA348 p9/44 | Test VitroHes | A | VitroHes | 4 ng/ml bFGF | MEF | 30 d Vitrohes | 100% day 15 | C, FIG. | | FIG. |
| | fr 070122 | with | | | | | | and day 22 | 40 | | 42A |
| | | Dexamethasone | | | | | | | | | |
| | | (DexM) | | | | | | | | | |
| | | (dissolved in | | | | | | | | | |
| | | DMSO) | | | | | | | | | |
| | | | B | VitroHes | 4 ng/ml bFGF + | MEF | 22 d Vitrohes + | 100% day 15 | C, FIG. | | FIG. |
| | | | | | 50 uM DexM | | 8 days | and day 22 | 40 | | 42A |
| | | | | | | | VitroHes/DexM | | | | |
| | | | C | VitroHes | 4 ng/ml bFGF + | MEF | 22 d Vitrohes + | 100% day 15 | C, FIG. | | FIG. |
| | | | | | 0.1% DMSO | | 8 days | and day 22 | 40 | | 42A |
| | | | | | | | VitroHes/DMSO | | | | |

TABLE 7

| | | | | | | | | Medium | | | Real- |
|---|---|---|---|---|---|---|---|---|---|---|---|
| Exp | Cell line | | | | Supplements/ | | | replacement | Study | Mor- | time |
| # | & passage | Aim | Groups | Medium | conc. | Surface | Duration | frequency | design | phology | PCR |
| 1 | SA002 p 57 | Maturation of | A | HCM | 2.5 mM NaB + | MEF | 21 d | 50% d 24 | D in | FIG. | FIG. |
| | fr 9/2-07 | HCLC with | | | 2.5 ng/ml HGF | | VitroHes + | (double conc. | | 41D | 42B |
| | | NaB + HGF | | | | | 5 d HMC | of NaB + | | | |
| | | | | | | | | HGF) | | | |
| | | | B | VitroHes | 4 ng/ml bFGF | MEF | 26 d | | D in | FIG. | FIG. |
| | | | | | | | VitroHes | | | 41C | 42B |
| 2 | SA167 p 32 | Maturation of | A | HCM | 2.5 mM NaB + | MEF | 21 d | 50% d 24 | D in | | FIG. |
| | fr 9/2-07 | HCLC with | | | 2.5 ng/ml HGF | | VitroHes + | (double conc. | | | 42B |
| | | NaB + HGF | | | | | 5 d HMC | of NaB + | | | |
| | | | | | | | | HGF) | | | |
| | | | B | VitroHes | 4 ng/ml bFGF | MEF | 26 d | | D in | | FIG. |
| | | | | | | | VitroHes | | | | 42B |

TABLE 8

| | | | | | | | | Media | | | Real- |
|---|---|---|---|---|---|---|---|---|---|---|---|
| Exp | Cell line | | | | Supplements/ | | | replacement | Study | Mor- | time |
| # | & passage | Aim | Groups | Medium | conc. | Surface | Duration | frequency | design | phology | PCR |
| 1 | SA002 p55 | Test medium | A | VitroHes | 4 ng/ml bFGF | MEF | 15 d | Rare: 50% at | E FIG. | | FIG. |
| | fr 070110 | from Cambrex, | | | | | Vitrohes + | d 23 | 43 | | 45 |
| | | HCM and | | | | | 15 days | | | | |
| | | frequency in | | | | | VitroHes | | | | |
| | | medium | | | | | | | | | |
| | | replacement | | | | | | | | | |
| | | | B | VitroHes | 4 ng/ml bFGF | MEF | 15 d | Frequent: 3 | E FIG. | | FIG. |
| | | | | | | | Vitrohes + | times a | 43 | | 45 |
| | | | | | | | 15 days | week | | | |
| | | | | | | | VitroHes | | | | |
| | | | C | HCM | 1% PEST | MEF | 15 d | Rare: 50% at | E FIG. | FIG. | FIG. |
| | | | | | 1% Glutamax | | Vitrohes + | d 23 | 43 | 44A | 45 |
| | | | | | 4 ng/ml bFGF | | 15 days | | | | |
| | | | | | | | HCM | | | | |
| | | | D | HCM | 1% PEST | MEF | 15 d | Frequent: | E FIG. | FIG. | FIG. |
| | | | | | 1% Glutamax | | Vitrohes + | 3 times a | 43 | 44B | 45 |
| | | | | | 4 ng/ml bFGF | | 15 days | week | | | |
| | | | | | | | HCM | | | | |

I. HCM-medium from Cambrex is a serum-free basal-medium supplemented with single aliquots of bovine serum albumin (BSA-FAF; 10 ml/500 ml HBM, cc-4362BB, Cambrex), ascorbic acid (0.5 ml/500 ml HBM, cc-4316BB, Cambrex), epidermal growth factor (rh-EGF; 0.5 ml/500 ml HBM, cc-4317BB, Cambrex), transferrin (0.5 ml/500 ml HBM; cc-4313BB, Cambrex), insulin (0.5 ml/500 ml HBM; cc-4321BB, Cambrex), hydrocortisone (0.5 ml/500 ml HBM; cc-4335BB, Cambrex) and antibiotics (GA-1000; 0.5 ml/500 ml HBM; cc-4381BB, Cambrex). The medium is optimised for primary hepatocyte cultures.

Protocols where VitroHES™ was replaced after day 15 or 23 with HCM-medium resulted in improved hepatocyte-like cells. The improved hepatocyte-like cells derived from cell-line SA002 and SA348 showed elevated gene expression levels of Albumin, CYP3A4 and UGT2B7 (FIG. 39). Judge by morphology the periphery of the colony with hepatocytes is wider in the improved cultures and the cells are less steatotic (FIG. 38). For study design see FIG. 37 for protocol details, see table 5 exp.1 and 2.

IIa. HCLC derived from cell line SA167, SA348, SA002 were differentiated according to the reference protocol however, supplementation with high concentration of Dexamethasone for 8 days at the end of the protocol improved the appearance of the HCLC (FIG. 41A+B). In addition, a trend was shown where elevated gene expression levels of HNF4alpha, Albumin, cyp3A4 and UGT2B7 was clear (FIG. 42A). For study design see FIG. 40, study design C and details of the protocols, see table 6, exp. 1, 2, 3.

IIb. Hepatocyte-like cells derived from cell line SA167 and SA002 were differentiated according to the reference protocol up to day 21. At that time point medium was replaced to HCM-medium supplemented with HGF and Sodium butyrate (NaB) for another 5 days, for details see table 7, exp 1, 2 and FIG. 40, study design D. The hepatocyte-like cells were improved by the new treatment judge by both morphology and real time PCR analysis. Less steatotic hepatocyte-like cells and a wider periphery of hepatocyte-like cells surrounding the colonies were appearing in colonies cultured in HCM-medium supplemented with HGF and NaB (FIG. 41C+D). In addition mRNA levels of Albumin, Cyp3A4, UTG2B7 and HNF4alpha were elevated in hepatocyte-like cells cultured according to the new protocol (FIG. 42B).

III. The importance of infrequent medium replacement for the differentiation and maturation process of hepatocyte-like cells derived from hBS cells was tested and analysed for several different mediums, among them VitroHES™ and HCM™-medium. The reference protocol was followed until day 15. After day 15 medium was either replaced three times a week or once a week. For study design see FIG. 43 and for details of the protocol see exp 1 in table 8. A clear trend was observed for all mediums tested. Rarely replacement of medium improved the hepatocyte-like cells according to morphology (FIG. 44A+B) and gene expression levels that were elevated for Cyp3A4, Albumin, UTG2B7 and HNF4alpha (FIG. 45). Data indicate that intrinsic factors are crucial for the differentiation process of the hBSC-derived hepatocyte-like cells.

### Example 19

### hBS Cell-Derived Hepatoblast Progenitor Cells

In order to establish a hepatoblast progenitor cell-line, 15 days old hBS-cell derived hepatoblast cells were isolated and reseeded on to different coatings. Prior to reseeding of hepatoblast-like cells, wells of a 96-well plate were pre-coated with the different coatings; mitomycin C-treated mEF-cells with a density of 17-20×10³ cells/cm², collagen I from rat tail (BD Biosciences, #354236) or Matrigel (basement membrane matrix, BD Biosciences, #356237) diluted 1:3 in HCM-medium. Coatings procedures as previous described in example 15 for collagen I and matrigel. The wells were filled with 50-100 μl of pre-warmed HCM-medium (HBM™, cc-3199 supplemented with HCM™ SingleQouts of cc-4316BB, cc-4362BB, cc-4335BB, cc-4313BB, cc-4321BB, cc-4317BB, cc-4381BB from Cambrex) supplemented with 20% FCS. Regions of hepatoblast-like cells from 15 days old cultures were micro dissected by using micro scalpels from BD and transferred with a stem cell knife to a collecting dish containing VitroHES™ medium. The micro dissected hepatoblast-like cells containing regions formed small clusters and were gathered from several plates. The clusters were washed with calcium and magnesium free PBS and treated with 5% Collagenase IV for for 5-15 min at 37° C. prior to reseeding onto different coatings of a 96-well plate containing HCM-medium supplemented with 20% FCS. After two days HCM-medium supplemented with 20% FCS was exchanged to serum-free HCM-medium. Cultures were provided with fresh HCM-medium every other day. After 5 days the cultures were fixed in 4% PFA and immunocytochemistry for HNF4alpha and CK19 was performed. Judge by morphology hepatoblast-like cells on collagen I and matrigel coatings had become hepatocyte-like cells with large polygonal and bi-nucleated cells (FIGS. 47 C and I), whereas hepatoblast-like cells reseeded on to mEF-cell layer did not show hepatocyte typical characteristics, rather clusters with hepatoblast-like cells with small nuclei were growing on the mEF-cell layer (FIG. 47 D-F and K, L. In addition the majority of the hepatoblast-like cell clusters grown on mEF were strongly positive for the hepatoblast marker CK19 (FIG. 47 J, K), where as only part of the hepatoblast-like cells on collagen I and matrigel were positive for CK19 (FIG. 47 G, H). Both hepatoblast-like cells on mEF-cell layer and hepatoblast-like cells on collagen I and matrigel coatings showed HNF4alpha positive nuclei, however the latter had larger nuclei and less strongly stained nuclei compared to hepatoblast-like cells grown on mEF (FIG. 47 A, B, D, E). The data indicate that mEF-cell layer have the ability to keep hepatoblast-like cells in a progenitor state while differentiation of hepatoblast-like cells into hepatocyte-like cells is allowed on collagen I and matrigel coatings.

### Example 20

### Functional Drug Transporters

Functional testing regarding transporters in hBSC-derived hepatocyte-like cells were analysed by applying indocyanine green (ICG) dye to the cultures. ICG was dissolved in distilled, sterile water to a concentration of 5 mg/ml and thereafter diluted in medium to a final concentration of 1 mg/ml. The ICG solution was added to the cell culture dish and incubated at 37° C. for approximately 60 minutes. After the incubation, the cells were rinsed with phosphate buffered saline (PBS), and the cellular uptake of ICG was examined with a stereo microscope. Hepatocyte-like cells were able to take up the ICG dye which suggests the presence of functional drug transporters within the cells (FIG. 48).

1. A cell population derived from hBS cells, wherein at least 20% of the cells in the cell population exhibit at least one of the following characteristics Alpha-1-antitrypsin, Cytokeratin 18, HNF-3beta, Albumin or Liver-Fatty-Acid-Binding-Protein and the cell population has at least three of the following six characteristics
A. Drug transporters
i) at least 1% of the cells exhibit protein and/or gene expression of BSEP,
ii) at least 1% of the cells exhibit protein and/or gene expression of MRP2,
iii) at least 1% of the cells exhibit protein and/or gene expression OATP-2 and/or OATP-8,
B. Drug metabolising enzymes
iv) at least 20% of the cells exhibit protein and/or gene expression of GST A1-1,
v) at least 20% of the cells exhibit protein and/or gene expression of at least 2 of the following CYP450s-1A2, -2A6, -2B6, -2C8, -2C9, -2C19-2D6, -2E1, -3A4 and -3A7,
vi) at least 20% of the cells do not exhibit protein and/or gene expression of GST P1-1.

2. A cell population according to claim 1, wherein the cell population has at least one of said drug transporter characteristics and at least one of said drug metabolism characteristics.

3. A cell population according to claim 1, wherein the cell population has at least four of said characteristics.

4. A cell population according to claim 1, wherein the cell population has all six of said characteristics.

5. A cell population according to claim 1, wherein at least 20% of the cells in the cell population exhibit at least one of the following characteristics Alpha-1-antitrypsin, Cytokeratin 18, HNF-3beta, Albumin or Liver-Fatty-Acid-Binding-Protein and the cell population has the following characteristics
A. Drug transporters
iii) at least 1% of the cells exhibit a functionally active OATP-2 and/or OATP-8
B. Drug metabolising enzymes
iv) at least 20% of the cells exhibit functional activity of GSTA1-1
v) at least 20% of the cells exhibit a functionally active Cyp1A2, Cyp3A4 and/or Cyp2C9 measured by analyzing the drug metabolites.

6. A cell population according to claim 1, wherein at least 75% of the cells in the cell population exhibit the following characteristics Alpha-1-antitrypsin, Cytokeratin 18, HNF-3beta, Albumin or Liver-Fatty-Acid-Binding-Protein and the cell population has at least the following characteristics
A. Drug transporters
iii) at least 10% of the cells exhibit a functionally active OATP-2 and/or OATP-8
B. Drug metabolising enzymes
iv) at least 30% of the cells exhibit functional activity of GSTA1-1
v) at least 50% of the cells exhibit a functionally active Cyp1A2, Cyp3A4 and/or Cyp2C9 measured by analyzing the drug metabolites.

7. A cell population according to claim 1, wherein characteristic i) is valid for at least 5% of the cells.

8. A cell population according to claim 1, wherein characteristic ii) is valid for at least 5% of the cells.

9. A cell population according to claim 1, characteristic iii) is valid for at least 5% of the cells.

10. A cell population according to claim 1, wherein characteristic iv) is valid for at least 30% of the cells.

11. A cell population according to claim 1, wherein characteristic v) is valid for at least 30% of the cells.

12. A cell population according to claim 1, wherein characteristic vi) is valid for at least 10% of the cells.

13. A cell population according to claim 1, wherein at least about 30% of the cells in the cell population express at least one of the following characteristics Alpha-1-antitrypsin, Cytokeratin 18, HNF-3beta, Albumin or Liver-Fatty-Acid-Binding-Protein.

14. A cell population according to claim 1, wherein at least about 5% of the cells co-express Cytokeratin 18 and CYP1A2.

15. A cell population according to claim 1, wherein at least about 5% of the cells co-express Cytokeratin 18 and CYP2A6.

16. A cell population according to claim 1, wherein at least about 5% of the cells co-express Cytokeratin 18 and CYP2B6.

17. A cell population according to claim 1, wherein at least about 5% of the cells co-express Cytokeratin 18 and CYP2C8, CYP2C9 and/or CYP2C19.

18. A cell population according to claim 1, wherein at least about 5% of the cells co-express Cytokeratin 18 and CYP2D6.

19. A cell population according to claim 1, wherein at least about 5% of the cells co-express Cytokeratin 18 and CYP2E1.

20. A cell population according to claim 1, wherein at least about 5% of the cells co-express Cytokeratin 18 and CYP3A4 and/or CYP3A7.

21. A cell population according to claim 1, wherein at least about 5% of the cells have at least one of the following additional characteristics
A. Receptor
vii) at least 5% of the cells exhibit protein and/or gene expression of c-Met,
B. Intercellular adhesion molecule
viii) at least 5% of the cells exhibit protein and/or gene expression of ICAM-1,
C. Drug metabolising enzyme
ix) at least 1% of the cells exhibit protein and/or gene expression of UGT,
D: Transcription factor
x) at least 90% of the cells exhibit no protein and/or gene expression of Oct-4.

22. A cell population according to claim 21, wherein the cell population has at least two of characteristics vii), viii), ix), or x).

23. A cell population according to claim 21, wherein the cell population has all four of characteristics vii), viii), ix), or x).

24. A cell population according to claim 21, wherein characteristic vii) is valid for at least 10% of the cells.

25. A cell population according to claim 21, wherein characteristic viii) is valid for at least 10% of the cells.

26. A cell population according to claim 21, wherein characteristic ix) is valid for at least 5% of the cells.

27. A cell population according to claim 21, wherein characteristic x) is valid for at least 10% of the cells.

28. A cell population according to claim 1, wherein the expression of at least one of the CYP450 proteins is inducible upon addition of an inducer.

29. A cell population according to claim 1, wherein the expression of GST A1-1 and/or GST M1-1 proteins are inducible upon addition of an inducer.

30. A cell population according to claim 21, wherein the expression of UGT protein is inducible upon addition of an inducer.

31. A cell population according to claim 28, wherein the inducer is selected from the group consisting of dexamethazone, omeprazole, alone or in combination.

32. A cell population according to claim 28, wherein the inducer comprises, Rifampicin, Dexamethasone, Desoxyphenobarbital, Ethanol, Omeprazole and Isoniazid.

33. A cell population according to claim 1, wherein the cell population exhibits enzymatic activity of at least one of the CYP450 proteins of characteristic v).

34. A cell population according to claim 1, wherein the cell population exhibits GST enzymatic activity.

35. A cell population according to claim 33, wherein the GST enzymatic activity is at least 0.4 μmol/min/mg of protein in a lysate of the cell population.

36. A cell population according to claim 21, wherein the cell population exhibits UGT enzymatic activity.

37. A cell population according to claim 1, wherein said cell population is cultured in vitro for at least one month with maintained characteristics.

38. A cell population according to claim 1, wherein said cell population is cultured in vitro for at least one week with maintained characteristics.

39. A cell population according to claim 1, wherein said cell population is cultured in vitro for at least 72 hours with maintained characteristics.

40. A cell population according to claim 1, wherein said cell population further expresses alpha-feto-protein.

41. A cell population according to claim 1, wherein said cell population is obtained in the presence of feeder cells such as human or mouse feeder cells.

42. A cell population according to claim 1, wherein said cell population is obtained in the absence of feeder cells.

43. A cell population according to 42, wherein said cell population is obtained using an extracellular matrix, wherein said matrix is of defined or undefined composition.

44. A cell population according to claim 42, wherein said cell population is obtained using a plastic cell culture vessel that is coated on the inside with one or more proteins, alone or in combination.

45. A cell population according to claim 44, wherein the one or more proteins are selected from the group consisting of collagen, laminin and combinations thereof.

46. A cell population according to claim 44, wherein said cell population is obtained using a 3D environment, such as a porous filter.

47. A cell population according to claim 41, wherein said cell population is xeno free.

48. A cell population derived from hBS cells, wherein at least about 10% of the cells in the cell population express at least one of HNF3beta and AFP and have proliferative capacity and the cell population has at least two of the following five characteristics:
A. Receptor
i) at least 1% of the cells exhibit protein and/or gene expression of alpha-6-integrin,
ii) at least 1% of the cells exhibit protein and/or gene expression of c-Met,
B. Intercellular adhesion molecule,
iii) at least 1% of the cells exhibit protein and/or expression of ICAM-1,
C. Transcription factor,
iv) at least 10% of the cells exhibit protein and/or expression of HNF-4 alpha.

49. A cell population according to claim 48, wherein the cell population has at least two of the following characteristics:
A. Receptor
i) at least 1% of the cells exhibit protein and/or gene expression of alpha-6-integrin,
ii) at least 1% of the cells exhibit protein and/or gene expression of c-Met,
B. Intercellular adhesion molecule,
iii) at least 1% of the cells exhibit protein and/or expression of ICAM-1,
C. Transcription factor,
iv) at least 10% of the cells exhibit protein and/or expression of HNF-4 alpha,
D. Cytokeratin
v) at least 1% of cells exhibits protein and/or expression of CK19,
vi) at least 1% of cells exhibits protein and/or expression of CK7,
E. Epithelial cell adhesion molecule
vii) at least 1% of cells exhibits protein and/or expression of EpCAM.

50. A cell population according to claim 48, wherein the cell population has at least three of said characteristics.

51. A cell population according to claim 48, wherein the cell population has at least four of said characteristics.

52. A cell population according to claim 48, wherein characteristic i) is valid for at least 5% of the cells.

53. A cell population according to claim 48, wherein characteristic ii) is valid for at least 5% of the cells.

54. A cell population according to claim 48, wherein characteristic iii) is valid for at least 5% of the cells.

55. A cell population according to claim 48, wherein characteristic iv) is valid for at least 15% of the cells.

56. A cell population according to claim 48, wherein at least about 15% of the cells in the population express at least one of HNF3beta and AFP and have proliferative capacity.

57. A cell population according to claim 48, wherein said cell population has at least one of the following characteristics
F. Drug transporters:
viii) at least 1% of the cells exhibit protein and/or gene expression of BSEP,
ix) at least 1% of the cells exhibit protein and/or gene expression of MRP2.

58. A drug discovery process comprising using the cell population of claim 1.

59. A method for studying drug transporters comprising using the cell population of claim 1 as an in vitro model.

60. A method for studying drug metabolizing enzymes comprising using the cell population of claim 1 as an in nitro model.

61. A method for studying hepatogenesis comprising using the cell population of claim 1 as an in vitro model.

62. A method for studying human hepatoregenerative disorders comprising using the cell population of claim 1 as an in vitro model.

63. A method for hepatotoxicity testing comprising using the cell population of claim 1 in an in vitro manner.

64. A medicament comprising the cell population of claim 1.

65. A method for the preventation and/or treatment of pathologies and/or diseases caused by tissue degeneration, such as, e.g., the degeneration of liver tissue, comprising administering an effective amount of the cell population of claim 1.

66. A method for the treatment of liver disorders comprising administering an effective amount of the cell population of claim 1.

67. A method for the prevention and/or treatment of liver disorders selected from the group consisting of auto immune disorders including primary biliary cirrhosis; metabolic disorders including dyslipidemia; type 2 diabetes; obesity; liver disorders caused by e.g. alcohol abuse; diseases caused by viruses such as, e.g., hepatitis B, hepatitis C, and hepatitis A; liver necrosis caused by acute toxic reactions to e.g. pharmaceutical drugs; and tumor removal in patients suffering from e.g. hepatocellular carcinoma comprising administering an effective amount of the cell population of claim 1.

68. A method for the treatment and/or prevention of metabolic pathologies and/or diseases comprising administering an effective amount of the cell population of claim 1.

69. A method for obtaining metabolically improved hepatocyte-like cells comprising using one or more cells of the cell population of claim 48.

70. A method for studying maturation towards hepatocyte-like cells comprising using one or more cells of the cell population of claim 48.

71. A method for screening a compound for hepatocellular toxicity, comprising exposing cells from a cell population as defined in claim 1 to the compound, and determine whether the compound is toxic to the cell.

72. A method for screening a compound for its ability to modulate hepatocellular function, comprising exposing cells from a cell population as defined in claim 1 to the compound, determining any phenotypic or metabolic changes in the cells that result from contact with the compound, and correlating the change with an ability to modulate hepatocellular function.

73. A method comprising the steps of
i) in vitro differentiating hBS cells or progenitors derived from hBS cells on a supporting matrix in a serum free medium for at least 5 days,
ii) changing the medium from about every 5 days to about every 25 days,
iii) isolating cells by mechanical isolation,
iv) optional dissociating the cells obtained in step iii) by treatment with an enzyme,
v) optional sorting the cells based on surface antigen expression,
in order to obtain a cell population as defined in claim 1.

74. A method according to claim 73, wherein the progenitors derived from hBS cells express at least one of HNF3beta and AFP and have proliferative capacity.

75. A method according to claim 73, wherein the serum free medium is VitroHES™ comprising bFGF.

76. A method according to claim 73, wherein the concentration of bFGF is from about 4 ng/ml to about 200 ng/ml.

77. A method according to claim 75, wherein the concentration of bFGF is at least 4 ng/ml.

78. A method according to claim 73, wherein the in vitro differentiation in step i) is performed for at least 10 days.

79. A method according to claim 73, wherein the supporting matrix comprises feeder cells, such as, e.g., human or mouse feeder cells.

80. A method according to claim 73, wherein the supporting matrix comprises an extracellular matrix of defined or undefined composition.

81. A method according to any of claims 80, wherein the supporting matrix comprises a coating comprising one or more proteins, alone or in combination, coating on the inside of a plastic cell culture vessel used for cell cultivation.

82. A method according to claim 73, wherein the supporting matrix comprises a 3D environment, such as a porous filter.

83. A method according to claim 82, wherein the porous filter has a pore size of about 4 μm in diameter.

84. A method according to claim 82, wherein the porous filter has been coated with one or more proteins, alone or in combination.

85. A method according to claim 81, wherein the one or more proteins are selected from the group consisting of collagen, laminin and combinations thereof.

86. A method according to claim 73, wherein step ii) is performed from about every 10 days to about every 20 days.

87. A method according to claim 73, wherein step ii) is performed every 14 to 15 days.

88. A kit comprising i) a cell population as defined in claim 1, ii) one or more maturation factors and/or a maturation culture medium, and iii) optionally, an instruction for use.

89. A kit according to claim 88, wherein the maturation culture medium is selected from the group consisting of VitroHES™, VitroHES™ supplemented with bFGF, autologuous pre-conditioned VitroHES™, and hepatocyte specific culture media.

90. A kit according to claim 88, wherein the one or more maturation factors are selected from the group consisting of bFGF, Epithelial Growth Factor, Hepatocyte Growth Factor and oncostatin M.

91. A kit according to claim 89, further comprising tools for monitoring maturation.

92. A kit according to claim 91, wherein the tools for monitoring maturation comprise
i) PCR primers against at least three, such as, e.g. at least four or at least five of the genes coding for expression markers selected from the group consisting of HNF3beta, AFP, albumin, BSEP, MRP2, OATP-2, OATP-8, GST A1-1, CYP450-1A2, CYP450-2A6, CYP450-2B6, CYP450-2C8, CYP450-2C9, CYP450-2C19 CYP450-2D6, CYP450-2E1, CYP450-3A4, CYP450-3A7, GST M1-1 and UGT, and
ii) a user's manual.

93. A kit according to claim 90, wherein the tools for monitoring maturation comprise
i) antibodies against at least three of the expression marker antigens selected from the group consisting of HNF3beta, AFP, albumin, BSEP, MRP2, OATP-2, OATP-8, GST A1-1, CYP450-1A2, CYP450-2A6, CYP450-2B6, CYP450-2C8, CYP450-2C9, CYP450-2C19 CYP450-2D6, CYP450-2E1, CYP450-3A4, CYP450-3A7, GST M1-1 and UGT, and
ii) a user's manual.

94. A cell population according to claim 1, wherein the cell population has at least five of said characteristics.

95. A cell population according to claim 21, wherein the cell population has at least three of characteristics vii), viii), ix), or x).

96. A cell population according to claim 49, wherein the cell population has at least three of said characteristics.

97. A cell population according to claim 48, wherein the cell population has at least five of said characteristics.

98. A cell population according to claim 48, wherein the cell population has at least six of said characteristics.
